(19) United States
(12) Patent Application Publication
(10) Pub. No.: US 2024/0299554 A1
(43) Pub. Date: Sep. 12, 2024

(54) PRODRUGS WITH 1-(DISULFANYL)ALKYLOXY-CARBONYL UNITS

(71) Applicant: PHARMACYTICS B.V., NIJMEGEN (NL)

(72) Inventors: Bouchra HAJJAJ, NIJMEGEN (NL); Somhairle MACCORMICK, NIJMEGEN (NL); Gerrit Herman VEENEMAN, NIJMEGEN (NL)

(21) Appl. No.: 18/572,705

(22) PCT Filed: Jun. 21, 2022
(86) PCT No.: PCT/NL2022/050353

§ 371 Date: Dec. 20, 2023

(30) Foreign Application Priority Data
Jun. 21, 2021 (NL) 2028506

(51) Int. Cl. A61K 47/54; C07C 381/04

(52) U.S. Cl. CPC A61K 47/549; A61K 47/54; C07C 381/04

(57) ABSTRACT

The invention is in the field of medical sciences. It provides new pharmaceutical methods and preparations. The invention relates to methods for improving the pharmacokinetic, physicochemical or pharmaceutical properties of drugs by converting the drug into a promoiety-containing 1-substituted disulfanylalkyl carbonate, thiocarbamate or carbamate prodrug. In particular, the invention relates to methods for improving the solubility, permeability, stability and/or oral bioavailability of a drug by converting the drug into a promoiety-containing 1-(disulfanylalkyl) carbonate, thiocarbonate or carbamate prodrug. The invention also provides new compositions comprising a drug covalently attached to a promoiety-containing 1-(disulfanylalkyl) carbonate, thiocarbonate or carbamate. More in particular, the invention relates to a method for increasing the oral bioavailability of a drug by covalently attaching a promoiety-containing 1-disulfanylalkyloxycarbonyl unit to a hydroxyl or amine containing drug in which the promoiety contains a 1-O—, 1-S—, 6-O— or 6-S-linked monosaccharide.

### TECHNICAL FIELD

The present invention relates to a compound, a reagent for making said compound, methods for preparing said compound and to the uses of said compound as a (mutual) prodrug, antedrug, medicament, therapy, imaging agent or diagnostic agent as well as to the use of said compound as a linker attached to a drug molecule to improve one or more of the following: solubility, permeability, stability, taste, oral bioavailability, dissolution and/or disposition or said drug molecule.

The invention is in the field of medical sciences. It provides new pharmaceutical methods and preparations. The invention relates to methods for improving the pharmacokinetic, physicochemical or pharmaceutical properties of drugs by converting the drug into a promoiety-containing 1-substituted disulfanylalkyl carbonate, thiocarbamate or carbamate prodrug. In particular, the invention relates to methods for improving the solubility, permeability, stability and/or oral bioavailability of a drug by converting the drug into a promoiety-containing 1-(disulfanylalkyl) carbonate, thiocarbonate or carbamate prodrug. The invention also provides new compositions comprising a drug covalently attached to a promoiety-containing 1-(disulfanylalkyl) carbonate or carbamate. More in particular, the invention relates to a method for increasing the oral bioavailability of a drug by covalently attaching a promoiety-containing 1-disulfanylalkyloxycarbonyl unit to a hydroxyl or amine-containing drug in which the promoiety contains a 1-O—, 1-S—, 6-O— or 6-S-linked monosaccharide.

The present invention relates to new compounds, in particular molecules that are prodrugs. The invention provides compositions and methods for using a substituted 1-(disulfanyl)alkyloxycarbonyl moiety to produce disulfanylalkyl carbonates, -thiocarbonates and-carbamates as well as 1-(disulfanyl)alkyl carbonates-thiocarbonates and-carbamates as tripartite prodrugs from hydroxy, thiol and amine-containing drugs to increase the solubility and permeability of said drug and to maximize the amount of an active drug that reaches the bloodstream and/or its site of action within the patient through alteration of the physicochemical, pharmacokinetic or pharmaceutical properties of the drug. The invention also relates to compositions and methods for using a substituted 1-(disulfanyl)alkyloxycarbonyl moiety as a prodrug to limit the occurrence of undesired effects of an active drug in exposed tissues that may not be the intended site of action, such as the gastrointestinal tract. The invention also relates to methods for increasing the oral availability of drugs by linking the drug to a substituted 1-(disulfanyl)alkyloxycarbonyl unit to obtain the compounds of the invention.

### BACKGROUND

A prerequisite of a drug is its ability to engage with its intended biological target with sufficient potency. At the same time, a drug should exhibit little side-effects as possible due to interaction with other biological entities, such as enzymes, receptors, ion channels and the like. However, having prerequisites do not make a molecule an acceptable drug. A drug also needs to be compatible with its intended administration route, including subcutaneous, intravenous, intramuscular, intraperitoneal, intrathecal, oral, buccal, sublingual, rectal, transdermal, intranasal, intrapulmonary, or ocular routes. For many drugs, oral applicability is desired or is even required. An ideal oral drug should therefore show sufficient solubility, stability to a range of pH's, ability to cross membranes, stability to intestinal and liver metabolism, resistance against excretion into bile or excretion by efflux transporters, partition into the target organ at therapeutic levels and limited or no partition to undesired tissues. However, many drugs fail to meet all these requirements to an acceptable extent, which will render said drug less effective. Optimization of the physicochemical, pharmaceutical, and pharmacokinetic properties of a drug is a key element in drug development.

Oral administration is among the most preferred routes to deliver medication to patients. However, inadequate oral bioavailability is a significant problem in the pharmaceutical world. Low oral bioavailability is associated with a lower efficacy and a variable patient response [see the publication of: Hellriegel, E. T., Clin. Pharmacol. Ther., 1996, 60, 601-7]. Drugs expressing low oral bioavailability are more difficult and costlier to transform into an acceptable formulation.

To compensate for low oral bioavailability a higher dose is generally required to realize the intended therapeutic effect, but a higher dose may also lead to a higher burden of dose-related side-effects, particularly in the gastrointestinal tract. In addition, a drug showing low oral bioavailability has a lower potential to be repositioned for new indications. Furthermore, several drug products are currently only available as injectable formulations and there is a great need for technologies that can facilitate reformulation of those drugs into effective oral applications.

Analysis of a large number of marketed drugs according to the Biopharmaceutics Drug Disposition Classification System (BDDCS) [Benet, L. Z., AAPS J., 2011, 13, 519-47] revealed that 40% of the marketed drugs show poor solubility (Class 2 and 4 drugs) whereas 30% of the marketed drugs show poor permeability as indicated by their poor metabolism (Class 3 and 4 drugs). It was further estimated that, from the drug candidates being investigated by the industry, up to 70% are poorly soluble class 2 compounds, while another 20% are not only poorly soluble but also poorly permeable and belong to class 4 compounds. It may therefore be concluded that the design of new chemical entities showing adequate oral bioavailability is becoming increasingly difficult.

Many remedies have been proposed to solve the problem of unsatisfactory oral bioavailability of drugs [see the publication of: Fasinu, P., Biopharm Drug Disp., 2011, 32, 185-209]. Proposed strategies for increasing the oral bioavailability include for instance solubilisation technologies, such as the use of different pharmaceutical acceptable salts, reduction of the particle size of the drugs, e.g. by micronisation or nanonisation techniques, the use of spray-dried dispersions and hot melt extrusion as well as the use of lipophilic liquids and semi-solid matrixes. None of these strategies appears universally applicable to resolve oral bioavailability problems for each drug and each time their potential need to be investigated on a case-by-case basis.

Another strategy to enhance drug oral bioavailability is the use of prodrugs [see the publication: Prodrugs and Targeted Delivery, Rautio, J, (Ed.), 2011, Wiley-VCH, Weinheim, Germany]. Prodrugs, which are inactive or less active derivatives of drug molecules and which undergo enzymatic or chemical transformation within the patient's body to regenerate the active form of said drug, have been a major strategy in meeting this challenge. By temporary modifying the physicochemical characteristics of drugs (e.g. by shielding of charges or by protection of ionization groups of said drug) prior to reaching their sites of action within the patient's body, prodrugs have a long history of overcoming physiologic barriers such as the gastrointestinal tract. In targeted cancer therapy, conventional chemotherapeutic agents, which lack intrinsic target specificity, are rationally modified to focus and redirect their cytotoxicity to tumor cells. The usefulness of many conventional, nonspecific chemotherapeutic agents, such as doxorubicin, paclitaxel, camptothecan, cisplatin, and their derivatives have been significantly extended by modification into prodrugs, particularly those harboring cell-targeting moieties.

Common subsets of prodrugs include solubilizing promoieties, such as amines, amino acids, carboxylates, phosphates, phosphonates, sulfates, sulfonates and the like, and promoieties that temporary mask polar functionalities, such as esters of carboxylates, phosphates and phosphonates. Furthermore, attachment of promoieties that either increase or decrease the lipophilicity of the drug can correct shortcomings of certain drugs. Another application area is the attachment of lipophilic chains as promoieties to facilitate lymphatic uptake.

Prodrugs can conceptually be divided into two categories, bioprecursor prodrugs and carrier prodrugs [see the publication: The Practice of Medicinal Chemistry, Ch. 31-32, Ed. Wermuth, Academic Press, San Diego, Calif., 2008]. Generally, bioprecursor prodrugs are compounds that are either inactive or have a low activity compared to the corresponding active drug compound but can be converted to an active form by metabolism or hydrolysis within the patient's body.

Carrier prodrugs are drug compounds that contain a promoiety, i.e. a covalently bound molecule that can have several purposes: a) the promoiety can transiently correct a specific suboptimal physicochemical property of a drug candidate, such as solubility and/or membrane permeability resulting in a higher oral bioavailability; b) the promoiety can delay generation of the active drug, e.g. in a slow release formulation; c) the presence of the promoiety can prevent gastrointestinal side-effects due to the action of the active drug on these tissues; d) the promoiety can block or slow down metabolism at a specific position. Such carrier prodrugs can be advantageous not only for orally administered drugs, but also for drugs that are administered intravenous, subcutaneous, intraperitoneal, rectal, transdermal, or intrathecal. Carrier prodrugs can also be beneficial for targeting a drug to specific tissues or cells as these category prodrugs can act as homing devices to direct a drug to a target tissue. Examples include Horizant, being a substrate for the monocarboxylate type 1 transporter, and Valganciclovir, being a substrate for the PEPT1 transporter. Other examples include the use of antibody-drug conjugates (ADCs) or the use of glucuronide-drug conjugates to target enzymes that are overexpressed in specific tumor cells. Promoieties can be attached directly by a covalent bond to a functional group of the drug (bipartite prodrugs) or can be attached indirectly through a linker molecule to the drug (tripartite prodrugs).

A special subset of carrier prodrugs are so-called drug-glycosides, in which a sugar part acts as the promoiety. In these prodrugs, the anomeric hydroxyl group of a sugar moiety is covalently linked to a drug molecule, either in a direct manner (e.g., to a hydroxyl of a drug), or in an indirect manner by use of a linker moiety that can be attached to any functional group in a drug. Sugar-carbamoylalkylidene drug conjugates were disclosed in WO2019121734 to improve oral bioavailability of the drugs. Although the oral bioavailability was enhanced, the efficiency of hydrolysis of the drug conjugates with the patient's body appeared to be dependent on the structural characteristics of the drug. Alternatively, the sugar moiety can be linked to a drug molecule through a non-anomeric hydroxyl group, e.g., the 6-OH of a sugar, again through a linker molecule. For example, 6-O-ketoprofen and indomethacin esters of glucose are known as well as a succinic ester linker between the 6-hydroxyl of galactose and dopamine. Although none of these conjugates were orally bioavailable and hence not usable for oral administration, they were able to pass the blood-brain barrier and facilitated accumulation of the drugs in brain.

Another special subset of a carrier prodrug are ADCs. These prodrugs are examples of bioconjugates and immunoconjugates and are an important class of highly potent pharmaceutical drugs designed as a targeted therapy for the treatment of patients with cancer. Unlike chemotherapy, ADCs are intended to target and kill only the cancer cells and spare healthy cells. ADCs are complex molecules composed of a monoclonal antibody linked to a biologically active cytotoxic payload or drug. The antibody part of the ADC specifically targets a certain tumor marker and track these proteins within the patient's body and attach themselves to the surface of cancer cells. The biochemical reaction between the antibody and the target protein triggers a signal in the tumor cell, which then absorbs or internalizes the antibody together with the cytotoxin. After the ADC is internalized, the cytotoxic drug is released and kills the cancer cell. Due to this targeting, ideally the drug has lower side effects and potentially gives a wider therapeutic window than other chemotherapeutic agents. The drug can be coupled to the antibody in various ways. In general, a linker is used as an interface between the drug and the antibody.

Further special classes of carrier prodrugs are mutual prodrugs (or codrugs) and antedrugs. In mutual prodrugs two pharmacologically active agents are linked together to form a single molecule. Each of these drugs then acts as a carrier for the other. Antedrugs are active drugs that become inactivated when part of the drug is hydrolyzed. This hydrolysable part can be compared to a promoiety in a prodrug.

### SUMMARY

It is an object of the present invention to provide new compounds that act as prodrugs and that comprise an improved linker, which compounds exhibit improved properties.

It is a further object of the present invention to provide a novel compound that can be used as a (mutual) prodrug, antedrug, medicament, therapy, imaging agent or diagnostic agent as well as to the use of said compound as a linker attached to a drug molecule to improve one or more of the following: solubility, permeability, stability, taste, oral bioavailability, dissolution and/or disposition of said drug molecule. With “use of said compound as a linker attached to a drug molecule” in the present invention, is meant that the compound according to the first aspect is a prodrug comprising the drug of which one ore more of the cited characteristics are to be improved.

In a first aspect, the invention relates to a compound according to Formula I or a pharmaceutically acceptable salt thereof according to claim 1. In another aspect, the present invention relates to a reagent compound—suitable for the preparation of the compound according to the present invention—according to Formula VIII according to claim 9. In another aspect, the present invention relates to a method for preparing said reagent compound according to claim 10. In another aspect, the present invention relates to a method for preparing a compound according to Formula Ia or a pharmaceutically acceptable salt thereof according to claim 11. In another aspect, the present invention relates to a method for preparing a compound according to Formula I or a pharmaceutically acceptable salt thereof according to claim 12. In another aspect, the invention relates to the compound according to the invention for use as a prodrug, a mutual prodrug, or an antedrug. In another aspect, the invention relates to the compound according to the invention for use as a medicament, therapy, imaging agent or diagnostic agent. In another aspect, the invention relates to the compound according to the invention for use as a linker attached to a drug molecule to improve one or more of the following: solubility, permeability, stability, taste, oral bioavailability, dissolution and/or disposition or said drug molecule.

Embodiments of these aspects are disclosed below and in the appended claims.

### List of Definitions

The following definitions are used in the present description and claims to define the stated subject matter. Other terms not cited below are meant to have the generally accepted meaning in the field.

“Alkyl” as used in the present description refers to an alkyl group that can be branched or unbranched. Examples of alkyl include methyl, ethyl, n-propyl, isopropyl, n-butyl, isobutyl, sec-butyl, t-butyl and n-pentyl.

“Alkoxy” as used in the present description refers to an alkyl group bonded to oxygen atom. Examples of alkoxy include methoxy, ethoxy and propoxy.

“Alkenyl” as used in the present description refers to a branched or unbranched hydrocarbon residue having at least one carbon to carbon double bond. Examples of alkenyl include ethenyl (vinyl), allyl, prop-1-enyl, but-1-enyl, but-2-enyl, but-3-enyl, 2-methyl-prop-2-enyl, pentenyl and hexenyl.

“Alkynyl” as used in the present description refers to a hydrocarbon residue having at least one carbon to carbon triple bond. Examples of alkynyl include ethynyl, propynyl, butynyl and pentynyl.

“Cyano” as used in the present description refers to: —CN.

“Amino” as used in the present description refers to: —NH₂.

“Amide” as used in the present description refers to: —C(═O)NH₂.

“Carbamate” as used in the present description refers to: —NH—C(═O)—O—.

“Urea” as used in the present description refers to: —NH—C(═O)—NH—.

“Carbonate” as used in the present description refers to: —O—C(═O)—O—.

“Cycloalkyl” as used in the present description refers to a saturated hydrocarbon ring structure. Examples of cycloalkyl include cyclopropyl, cyclobutyl, cyclopentyl, cyclohexyl, cycloheptyl and cyclooctyl.

“Cycloheteroalkyl” as used in the present description refers to a saturated hydrocarbon ring structure having one or more heteroatoms, such as O, N or S, within the ring. Examples of cycloheteroalkyl include azetidine, oxetane, pyrrolidine, tetrahydrofuran, piperidine, tetrahydropyran, piperazine and morpholine.

“Cycloalkenyl” as used in the present description refers to a partially saturated hydrocarbon ring structure. Examples of cycloalkenyl include cyclobutenyl, cyclopentenyl and cyclohexenyl.

“Heterocycle” refers to an aromatic, saturated or partially saturated ring structure having 3 to 6 carbon atoms and 1 to 4 heteroatoms (such as N, S, and O). Examples of heterocycle include thienyl, furyl, pyranyl, pyrrolyl, imidazolyl, pyrazolyl, isothiazolyl, isoxazolyl, pyridyl, pyrazinyl, pyrimidinyl, pyridazinyl, oxetanyl, tetrahydrofuranyl, tetrahydropyranyl, triazolyl, tetrazolyl, oxazolyl, oxadiazolyl, pyrrolinyl, piperidinyl and morpholinyl.

“Aryl” as used in the present description refers to an aromatic hydrocarbon ring. Examples of aryl include phenyl and naphtyl.

“Drug” of “drug molecule” as used in the present description refers to a pharmaceutically active agent. This can be an approved drug of medicament, or a candidate drug undergoing laboratory testing, or preclinical or clinical trials.

“Drug moiety” as used in the present description refers to a drug molecule that is part of/attached to the compounds according to present invention.

“Amino acid” as used in the present description refers to natural L-amino acids and their D-counterparts and it refers also to homo amino acids and to unnatural amino acids derived from the glycine or R-alanine core structures.

“Sugar” as used in the present description refers to alpha- and beta-linked monosaccharides, disaccharides, trisaccharides and tetrasaccharides. Mono-saccharides have the general molecular formula (CH₂O)ₙ, where n can be 4, 5 or 6. They can be classified according to the number of carbon atoms in a molecule. Monosaccharides where n is 4 are referred to as tetroses, where n is 5, these are referred to as pentoses, e.g., ribose and deoxyribose, and where n is 6, these are referred to as hexoses, e.g., mannose, glucose and galactose. Disaccharides are made up of two monosaccharide units. Examples of relevant disaccharides are maltose, isomaltose, cellobiose, gentiobiose and lactose. Tri- and tetrasaccharides are oligosaccharides composed of three and four monosaccharide units, respectively. Examples of trisaccharides are maltotriose, isomaltotriose, negerotriose and melezitose. Examples of tetrasaccharides are maltotetraose and nigerotetraose.

“Vitamin” as used in the present description refers to vitamin A, B1-9, C, D2-3 and E.

“Antibody” as used in the present description refers to glycoproteins belonging to the immunoglobulin superfamily and have an average molecular weight of 150 kDa. They are typically made of basic structural units—each with two large heavy chains and two small light chains. There are several different types of antibody heavy chains that define the five different types of crystallizable fragments (Fc) that may be attached to the antigen-binding fragments. The five different types of Fc regions allow antibodies to be grouped into five isotypes. Though the general structure of all antibodies is very similar, a small region at the tip of the protein is known as the hypervariable region, allowing millions of antibodies with slightly different tip structures, or antigen-binding sites, to exist. Each of these variants can bind to a different antigen.

“Protein” as used in the present description refers to large biomolecules, or macromolecules, consisting of one or more long chains of amino acid residues. These biomolecules may also contain oligosaccharide chains.

“Oral bioavailability” as used in the present description refers to the extent and rate at which a drug enters the systemic circulation after oral administration, thereby becoming available to access the site of desired action. Oral bioavailability in the context of the present invention is herein defined as the fraction of an orally administered drug that reaches the systemic circulation.

“Prot” or “protective group” as used in the present description refer to groups that are a reversibly formed derivative of an existing functional group in a molecule, such as an —OH group. The protective group is temporarily attached to decrease reactivity of the functional group so that the protected functional group does not react under synthetic conditions to which the molecule is subjected in one or more subsequent steps. In the present invention, compounds having one or more of these protective groups are precursor compounds and in case the compound according to the invention is e.g. for a further synthesis, e.g. as a promoiety for a prodrug, these one or more protective groups need to be removed for the final compound obtained (e.g. the prodrug) to be active.

“LC-MS/MS methods” as used in the present description refers to a tool for the detection of residual chemical compounds, confirmatory identification of small organic molecules, and confirmation and quantitation of contaminants and adulterants in pharmaceutical and food samples. The abbreviation stands for Liquid Chromatography with tandem Mass Spectrometry.

### BRIEF DESCRIPTION OF DRAWINGS

FIGS. 1A and 1B are graphs showing the release of the drug Cinacalcet after an in vitro treatment of Cinacalcet conjugate 102 (not according to the invention) with glutathione. FIG. 1A shows the peak area for the formation of Cinacalcet and other intermediates/adduct versus time. FIG. 1B represent the concentration of Cinacalcet conjugate 102 and Cinacalcet versus time. The presence of the compounds was followed at 254 nm by LC-MS.

FIGS. 2A and 2B are graphs showing the release of the drug Duloxetine after an in vitro treatment of Duloxetine conjugate 103 (not according to the invention) with glutathione. FIG. 2A shows the peak area for the formation of Duloxetine and other intermediates/adduct versus time. FIG. 2B represent the concentration of Cinacalcet conjugate 103 and Duloxetine versus time. The presence of the compounds was followed at 254 nm by LC-MS.

FIGS. 3A and 3B are graphs showing the release of the drug Cinacalcet after an in vitro treatment of Cinacalcet conjugate 60 (according to the invention) with glutathione. FIG. 3A shows the peak area for the formation of Cinacalcet and other intermediates/adduct versus time. FIG. 3B represent the concentration of Cinacalcet conjugate 60 and Cinacalcet versus time. The presence of the compounds was followed at 254 nm by LC-MS.

FIGS. 4A and 4B are graphs showing the release of the drug Duloxetine after an in vitro treatment of Duloxetine conjugate 61 (according to the invention) with glutathione. FIG. 4A shows the peak area for the formation of Duloxetine and other intermediates/adduct versus time. FIG. 4B represent the concentration of Duloxetine conjugate 61 and Duloxetine versus time. The presence of the compounds was followed at 254 nm by LC-MS.

FIGS. 5A-D show tables 2a-1, 2a-2, 2a-3, and 2b, respectively.

FIGS. 6A-B show tables 3-1 and 3-2, respectively.

FIGS. 7A-I show tables 4-1, 4-2, 4-3, 4-4, 4-5, 4-6, 4-7, 4-8, and 4-9, respectively.

### DESCRIPTION OF EMBODIMENTS

The present invention will be disclosed in more detail below.

An essential feature of a carrier prodrug is its ultimate cleavage into the active parent drug. In many cases this cleavage process is achieved, among others, by esterases, peptidases, proteases, phosphatases, glycosidases and glucuronidases, but, depending on the nature of the prodrug, can also be accomplished by reducing agents (e.g. glutathione, reductases) or CYP450 enzymes. If the aim is to increase bioavailability it is most desirable that deconjugation to the parent drug proceeds fast to avoid accumulation of metabolites with unknown and potentially unwanted properties. Also, after absorption, the prodrug may be cleared faster than the parent drug.

In many situations a linker molecule is used as part of the prodrug. A linker molecule is defined as a covalently bound molecular interface between a functional group of a drug and a promoiety. Taken together, the promoiety, the linker and the drug are categorized as so-called tripartite prodrugs. Tripartite prodrugs can be subdivided into several classes, based on the disintegration properties of the linker.

Type A—tripartite prodrugs with self-immolative linkers (they will be cleaved between promoiety and linker): these are linkers that spontaneously disintegrate via end-to-end decomposition or cyclization mechanisms. The drug is conjugated to the proximal end of the linker, whereas the distal end—the end that initiates the decomposition—contains a promoiety to prevent the disintegration of the linker. Removal of the promoiety (often termed a trigger) initiates the decomposition process. Self-immolation results in the scission of bonds at the proximal end of the linker, resulting in the release of the conjugated drug. Examples of Type A—tripartite prodrugs are those containing 4-aminobenzyl- and 4-hydroxybenzyl-type linkers.

Type B—tripartite prodrugs have linkers that require chemical or enzymatic hydrolysis of the linkage between the linker and the drug (they will be cleaved between linker and drug). In certain cases, this process is accelerated if the linkage between the promoiety and the linker is hydrolyzed first. Examples are ester-type linkages between the linker and a hydroxyl group of a drug and optionally to the promoiety.

Type C—tripartite prodrugs having self-immolative linkers that do not require cleavage at the distal site but are cleaved internally to produce an unstable linker intermediate that disintegrate spontaneously, liberating the parent drug. In this case, prior hydrolysis of the linkage between the linker and the promoiety is not required. Examples of these linkers are specific peptidase-sensitive dipeptide bonds and glutathione or disulfide reductases-sensitive 2-disulfanylethyl carbonates. Tripartite prodrugs of Type C hold great potential because these molecules do no longer rely on specific cleavage of the bond between the promoiety and the linker and/or the cleavage of the bond between the linker and the drug. It is the aim of the present inventor to provide improved Type C tripartite prodrugs.

Without being bound to theory, the hydrolysis of the linker itself can be expected to be less dependent on the chemical environment caused by the promoiety and the drug resulting in a more predictable liberation of the drug. In the prior art have been reported 2-disulfanylethyl carbamates [see publication: Bioconjugate Chem., 2017, 28, 2086] as suitable linkers in the context of antibody-drug conjugates (wherein the antibody is the promoiety) since these 2-disulfanylethyl carbamates linkers were found to be hydrolyzed after lysosomal absorption and degradation of the ADCs. It has been proposed in literature that enzymatic or glutathione-mediated reduction of the disulfide leads to an unstable 2-mercaptoethyl carbamate having self-immolative properties resulting in the formation of the parent drug compound. However, various literature reports have indicated that the 2-mercaptoethyl carbamate intermediates are more stable than was anticipated and that these hydrolyze relatively slowly with half-lives exceeding 1 h, to the parent drug [see for example publications: J. Med. Chem., 2018, 61, 4904; Bioorg. Med. Chem. Lett., 2006, 16, 5093]. It has been observed in literature that reduction of the disulfide group is dependent on the concentration of glutathione. In cells, glutathione may reach concentrations up to 10 mM, while in blood and plasma the concentration is only in the μM range.

As shown in more detail in the Examples below, the present inventors have tested the use of 2-disulfanylethyl carbamate linkers for suitability to improve the oral bioavailability (see the examples not according to the invention Cinacalcet and Duloxetine). However, the inventors observed that the apparent unpredictable properties make the use of 2-disulfanylethyl carbamate linkers less suitable in the context of oral administration of drug-conjugates, for instance to improve the oral bioavailability of the parent drug.

In view of the limited number of endeavors towards tripartite prodrugs of Type C for oral application, there remains a need for improved methods and ways to identify and prepare these types of linkers that can be used to improve one or more of the physicochemical, pharmaceutical, or pharmacokinetic properties of a drug.

The present inventors have discovered that substituted 1-(disulfanyl)alkyl carbamate linker-type prodrugs according to the present invention can be applied as effective tripartite prodrugs of type C. These prodrugs are unprecedented and can bring advantages with respect to solubility, permeability and/or oral bioavailability of hydroxyl, thiol and amine containing drugs. As shown in the Examples according to the invention analogues of Cinacalcet and Duloxetine were prepared both with 1-(disulfanyl)ethyl carbamate linkers not according to the invention (compounds 102 and 103 respectively) and with 1-(disulfanyl)methyl carbamate linkers according to the invention (compounds 60 and 61 respectively) which showed much better results. This clearly shows the effect of the present invention and the large difference that a single methyl group in the linker makes.

The linkers according to the present invention lead to a faster and much more efficient release of the drug.

In vivo pharmacokinetic studies have learned that prodrugs according to the present invention are readily converted into the parent drug. Without wanting to be bound by a particular theory, cleavage of the S—S bond is expected to result in the formation of an unstable 1-sulfanylalkylidene carbamate or carbonate intermediate, which decompose readily to produce the active drug. These features have significant advantages over the previously mentioned prodrugs.

In a first aspect, the present invention relates to a compound according to Formula I or a pharmaceutically acceptable salt thereof:

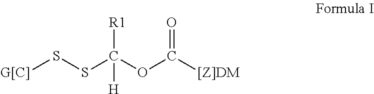

wherein each solid line represents a covalent bond, wherein H is hydrogen, O is oxygen, C is carbon, S is sulfur, and C═O is a carbonyl group; wherein R1 is selected from the group consisting of hydrogen, methyl, ethyl, n-propyl, isopropyl, n-butyl, sec-butyl, and t-butyl, preferably R1 is hydrogen or methyl; wherein G is an organic structure and [C] represents a carbon atom of G; wherein DM is a drug moiety and [Z] represents a part of DM; and wherein Z is selected from the group consisting of O, S, and N.

These types of compounds provide the effect of the present invention, specifically because of the methylene type linker (—CH(R1)-) between the disulfide (S—S) and the carbamate (C—C(═O)—Z) parts of the structure.

In an embodiment, Z is O—C representing an oxygen and a carbon atom of drug moiety DM in which the oxygen atom O is covalently bound to the carbonyl group of the compound of Formula I and wherein the carbon atom C is covalently bound to O and to three hydrogen atoms and/or carbon atoms of DM. In this embodiment the original drug molecule (DM-ZH) comprises an alcohol function —OH that is coupled via said alcohol function to the linker. Said alcohol function may be a phenol. This type of linker will provide a ˜C—O—C(═O)—O—C˜ type linkage which is called a carbonate type linkage.

In an embodiment, Z is N—C representing a nitrogen and a carbon atom of drug moiety DM in which the nitrogen atom N is covalently bound to the carbonyl group of the compound of Formula I and wherein nitrogen atom N and carbon atom C are covalently bound to each other and to respectively one and three hydrogen atoms and/or carbon atoms of DM. In this embodiment the original drug molecule (DM-ZH) comprises a primary or secondary amine function that is coupled via said amine function to the linker. This type of linker will provide a ˜C—O—C(═O)—N—C˜ type linkage which is called a carbamate type linkage.

In an embodiment, Z is S—C representing a sulfur and a carbon atom of DM in which sulfur atom S is covalently bound to the carbonyl group of the compound of Formula I and wherein carbon atom C is covalently bound to sulfur atom S and to three hydrogen atoms and/or carbon atoms of DM. In this embodiment the original drug molecule (DM-ZH) comprises a thiol function that is coupled via said thiol function to the linker. This type of linker will provide a ˜C—O—C(═O)—S—C˜ type linkage which is called a thiocarbonate type linkage.

In an embodiment, Z is O—N representing an oxygen and a nitrogen atom of DM in which oxygen atom O is covalently bound to the carbonyl group of the compound of Formula I and wherein nitrogen atom N is covalently bound to oxygen atom O and to two hydrogen atoms and/or carbon atoms of DM. In this embodiment the original drug molecule (DM-ZH) comprises a hydroxylamine or hydroxamic acid function that is coupled via said hydroxylamine or hydroxamic acid function to the linker. This type of linker will provide a ˜C—O—C(═O)—O—N˜ type linkage which is called an amino carbonate-type linkage.

The promoiety used in the present invention is organic group G[C]. The linker used in the present invention is a so-called 1-substituted-(disulfanyl)alkyloxycarbonyl moiety, also called a disulfide-alkylene-carbonate linker [—S—S—CH(R1)-O—C(═O)—]. The organic group G (or G[C]) may be selected from a covalently bound group of saturated and unsaturated, cyclic and noncyclic, aromatic and non-aromatic organic structures containing C and H atoms and optionally containing one or more N, O, F, Cl, Br, I, B, P, and S atoms with the provision that the disulfide is always covalently attached to a primary, secondary or tertiary carbon atom C in G, and preferably with a further provision that this particular carbon atom C does not contain an OH, SH or NH group, a double bonded oxygen or a double bonded sulfur. G can vary in size from a methyl to an antibody, the latter having an average molecular weight up to 150 kDa. G may optionally contain one or more of the isotopes ¹³C, ¹⁴C, D, T, ¹⁸F, ¹³¹I, ¹⁸O, or ³²P. G may also contain carboxylates, phosphates, phosphonates, sulfates and sulfonates and metals, such as Li⁺, Na⁺, K⁺, Ca²⁺ and Mg²⁺ as counter ions of these charged functionalities. The carbonyl of the 1-substituted-(disulfanyl)alkyloxycarbonyl moiety is covalently bound to the functional group ZH of a drug molecule (DM-ZH) representing small molecules to therapeutic peptides, to provide the compounds according to the present invention. ZH is part of the drug molecule and represents an alcohol, phenol, oxime, a primary or secondary amine or a thiol, with the provision that NH and NH₂ are not part of an amide, carbamate or urethane within said drug molecule. “DM” denotes a drug moiety which forms with one of its OH, NH₂, NH or SH functional group, the active drug; it is to be understood that in an embodiment, the carbonyl moiety of the 1-substituted-(disulfanyl)alkyloxycarbonyl is linked to the OH, NH₂/NH or SH group of the active drug to form a carbonate-, carbamate- or thiocarbonate-type linkage. The drug moieties with its attached OH, SH, NH₂ or NH functional groups preferably have a molecular weight in the range of 100-1000 daltons.

The invention can be applied to many drugs but may be applied especially to drugs that have one or more imperfections, such as poor solubility, permeability, (oral) bioavailability or dissolution rate, or the induction of gastrointestinal side effects, undesired metabolism or bad taste. In those cases, it is preferred that G is selected from a structural motif that optimizes at least one of these drug imperfections.

In a specific embodiment, G is selected from the group of C1-20 alkyl, C1-20 heteroalkyl, polyethyleneglycol, 4-, 5-, 6-, 7- or 8-membered cycloalkyl, or heterocyclic alkyl, C1-20 alkenyl, heteroalkenyl, alkynyl, or heteroalkynyl, an aryl or heteroaryl moiety or combinations of these elements, optionally diversified with one or more hydroxy, alkoxy, acyl esters, non-substituted-, monosubstituted- and disubstituted amine, amide, carbamate, carbonate, urea, halogen, nitrile, CF₃, one or more carboxylates, primary, secondary or tertiary amines, cyclic amines, hydroxyls, alkoxy's, phosphates, phosphonates, sulfates, sulfonates, boronates, polyethyleneglycols, L or D-amino acids, L or D-homoamino acids, dipeptides, tripeptides, polypeptides, C1-24 alkyl or alkenyl chains, lipids and fatty acids, 1-O—, 1-S, 6-O— or 6-S-linked hexose sugars, vitamins, 1-O-linked glucuronic acids, a covalently linked protein or antibody, either directly bound or indirectly linked through a spacer. If the amino acid, peptide or sugar already contains a free thiol group, this functionality can be connected directly such that it becomes part of S—S linkage of the 1-substituted-(disulfanyl)alkyloxycarbonyl prodrug. In an embodiment, G is a sugar, more preferably an alpha- or beta-linked monosaccharide, more preferably a hexose, even more preferably selected from the group consisting of a D-glucose and D-galactose or their partially deoxygenated or OH-substitution variants. With partially deoxygenated monosaccharide is meant C-2, C-3, C-4 or C-6 deoxy variants. One or two of the hydroxyls of the sugar can be optionally replaced by one or two alkoxy, hydrogen or fluoride.

Most preferred sugars are β-D-glucose and β-D-galactose and their partially deoxygenated or OH-substitution variants. If G represents a protein or an antibody, it can either be connected indirectly through a bridging molecule bound to any functional group of the antibody or directly, for example through the thiol group of a cysteine residue present in the antibody which then becomes part of S—S linkage in the 1-substituted-(disulfanyl)alkyloxycarbonyl conjugate. Such a bridging molecule can be a bifunctional structure, having a heteroalkyl chain of 3-10 atoms, containing an SH functional group and a suitable moiety to form a covalent bond with a D- or L-amino acid, a peptide varying in size from 2 up to 40 amino acids, a sugar, or a vitamin. An example of a suitable bridging molecule is thioglycolic acid, structurally one of the simplest bridging molecules. ZH represents an alcohol, thiol, a primary or secondary amine; it is to be understood that ZH is an integral part of the selected drugs exemplified by DM-ZH.

Representative amine-containing drugs that can be used in the compounds according to the present invention include 5′-Deoxy-5-fluorocytidine, Cytarabine, Lenalidomide, Thalidomide, Acyclovir, Doxorubicin, Losartan, Orciprenaline, Albendazole, Duloxetine, Mesalazine, Linagliptin, Atomoxetine, 5-Fluorouracil, Methylphenidate, Palbociclib, Azacitidine, Gabapentin, Metoprolol, Nintedanib, Carvedilol, Gemcitabine, Rasagiline, Pscilocin, Celecoxib, Ibrutinib, Riluzole, Meropenem, Cinacalcet, Lapatinib, Tamiflu, and Ceftriaxon.

Representative hydroxy-containing drugs that can be used in the compounds according to the present invention include Abiraterone, Fesoterodine, Rotigotine, Ciclopirox, Acyclovir, Fulvestrant, Tenofovir, Azacitidine, Ganciclovir, Testosterone, Cytarabine, Kalydeco, Tizoxanide, Cannabidiol, Paliperidone, Venlafaxine, Edaravone, Paracetamol, Vorinostat, Gemcitabine, Paclitaxel, Pscilocin, Estradiol, Propofol, and Orciprenaline. Representative thiol-containing drugs that can be used in the compounds according to the present invention include mercaptopurin, acetylcysteine, bucillamine, captopril, and zofenoprilat.

In an embodiment, the drug DM-ZH is selected from the group consisting of Abiraterone, Cinacalcet, Duloxetine, Ritalin and Mercaptopurin.

G may be selected from the group of drug-optimizing elements, defined as chemical structures that are attached to the drug, to optimize one or more of its physicochemical, pharmacokinetic or pharmaceutical imperfections. The drug-optimizing chemical structure G is covalently attached to a 1-(disulfanyl)alkyloxycarbonyl linker through a carbon (a C—SS linkage) which in turn is covalently bound to an active drug. According to this definition, the 1-(disulfanyl)alkyloxycarbonyl moiety is to be understood as a linker that connects an active drug to a drug-optimizing chemical structure. Depending on the drug and its imperfections, the drug-optimizing element can be as simple as a short alkyl or modified alkyl group, for instance to increase or decrease lipophilicity of a drug or to lower the crystal energy to facilitate dissolution. The drug-optimizing chemical structure can also be a solubility or permeability enhancing moiety that contains acidic, basic, or hydrophilic groups, such as carboxylates, amines, sulfates, sulfonic acids, phosphates, phosphonates, hydroxyls, amino acids, sugars, and combinations of these variants. Solubility enhancing moieties such as carboxylates, phosphates, sulfates, or amines have been reported earlier but have some drawbacks, such as instability in solution (e.g., hemisuccinate esters), slow or incomplete hydrolysis after absorption (e.g., alcohols, sulfates, amino acids, sugars). These drawbacks do not occur with the substituted 1-(disulfanyl)alkyloxycarbonyl prodrugs as these molecules are stable and do not depend on hydrolytic enzymes. Instead, these prodrugs are readily cleaved by glutathione or by disulfide-reducing proteins. Depending on the structural features of the drug and its physicochemical, pharmacokinetic or pharmaceutical issues, the drug-optimizing chemical structure can also contain a lipophilic moiety or a tissue- or cell-targeting commodity, such as an amino acid, dipeptide or tripeptide, a sugar, a vitamin, a substrate for a membrane transporter, a receptor, an enzyme or an antibody.

In a first embodiment, G[C] is represented by Formula IIa:

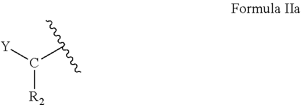

wherein Y is selected from the group consisting of compounds according to Formulas IIIa, IIIb, IIIc, IIId, and IIIe below:

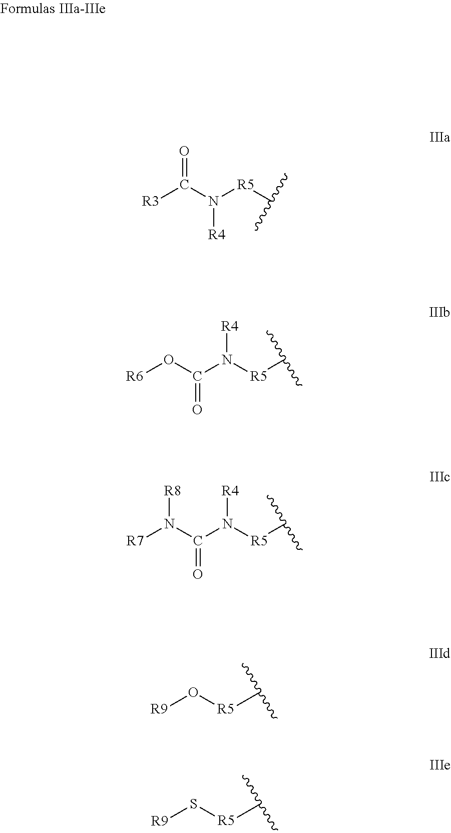

wherein R2 is hydrogen or methyl; wherein R3, R6, and R9 are each independently a C1-20 (hetero)alkyl or a saturated or unsaturated 3-8 membered (hetero)cyclic structure; wherein R4 is a hydrogen or a C1-6 (hetero)alkyl; wherein R5 is selected from the group consisting of a bond, a C1-8 (hetero)alkyl, C1-8 (hetero)alkenyl, C1-8 (hetero)alkynyl, and a saturated or unsaturated 3-8 membered (hetero)cyclic structure; and wherein R7 and R8 are independently selected from the group consisting of hydrogen, a C1-20 (hetero)alkyl, C1-20 (hetero)alkenyl, C1-20 (hetero)alkynyl, and a saturated or unsaturated 3-8 membered (hetero)cyclic structure; or

wherein G[C] is represented by Formula IIb:

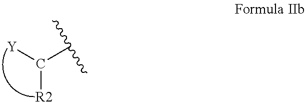

wherein Y and R2 together form a saturated or unsaturated 3-8 membered (hetero)cyclic structure.

In another embodiment, G[C] is represented by Formula IV:

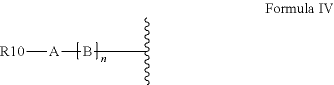

wherein R10 is selected from the group consisting of a carboxylate, hydroxyl, phosphate, phosphonate, sulfate, sulfonate, R11 N(R12)-, NH₂CH(R13)C(═O)NH—, a 3-6 membered (hetero)cyclic ring, for example an azetidine, a pyrrolidine, or a piperidine rig, and a sugar; wherein A is selected from the group consisting of a bond, —CH₂—, —CH(NH₂)—, —CH₂CH₂—, —C(CH₂OH)H—, —CH₂CH(OH)—, and —C(═O)NH—; wherein B is selected from the group consisting of —CH₂—, —O—CH₂—, —CH₂CH₂—O—, and —O—CH₂CH₂—; wherein n is an integer from 1-20; wherein R11 and R12 are independently selected from the group consisting of hydrogen, a C1-20 (hetero)alkyl, C1-20 (hetero)alkenyl, C1-20 (hetero)alkynyl, and a saturated or unsaturated 3-8 membered (hetero)cyclic structure; and wherein R13 is selected from the group consisting of hydrogen, methyl, ethyl, propyl, isopropyl, sec-butyl, isobutyl, benzyl, 4-hydroxybenzyl, 2-methylthioethyl, hydroxymethyl, 4-aminobutyl, 3-aminopropyl, —CH₂—CH₂—CO—NH₂, —CH₂—CO—NH₂, —CH₂—CH₂—COOH, —CH₂—COOH, —CH₂—CH₂—CH₂—HN—(HN)═C(NH₂), and —CH₂-cycl(C═CH—N═CH—NH).

In another embodiment, G[C] is selected from the group consisting of the following structures:

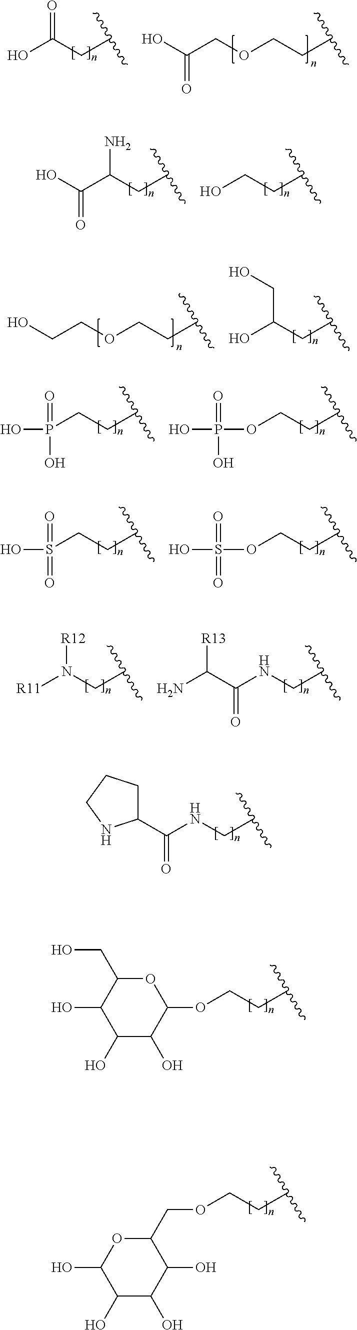

In a preferred embodiment, the compound according to the present invention is selected from the group consisting of the following compounds:

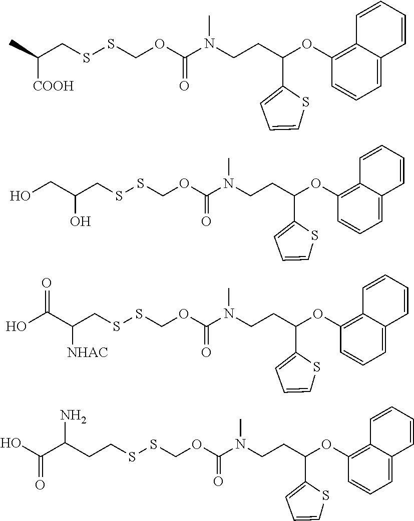

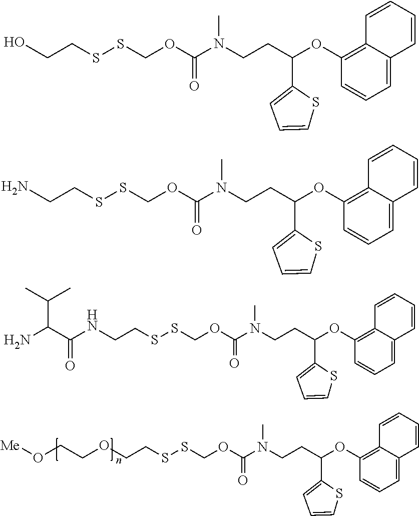

In an embodiment, G[C] is represented by Formula V:

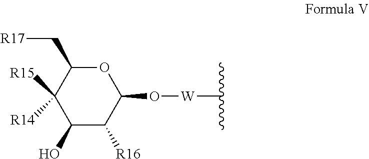

wherein W is selected from the group consisting of C1-20 (hetero)alkyl, —C(═O)N(R18)R19, —C(═O)NR20, and —C(═O)N(R18)-CH₂—O—(CH₂)ₘ—; wherein R14 and R15 are each independently selected from OH, F, and H with the proviso that if one of R14 or R15 is OH, the other is H; wherein R16 is OH or F; wherein R17 is selected from the group consisting of OH, F and H; wherein R18 is selected from the group consisting of methyl, ethyl, n-propyl, isopropyl, n-butyl, sec-butyl, iso-butyl and 2-methoxyethyl; wherein R19 is a C1-10 (hetero)alkyl; wherein NR20 is a (hetero)cyclic structure; and wherein m is an integer between 2 and 6. More preferred, O—W is selected from the group consisting of the following structures:

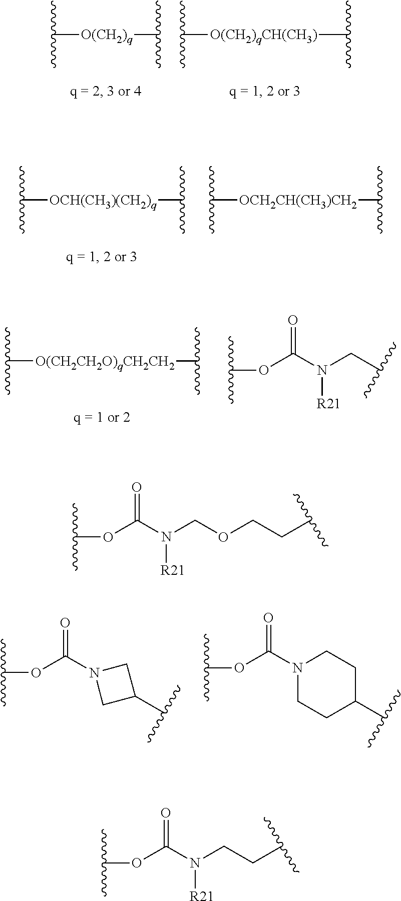

wherein R21 is selected from the group consisting of methyl, ethyl, n-propyl, isopropyl, n-butyl, isobutyl, sec-butyl, and 2-methoxyethyl.

In an embodiment, G[C] is represented by Formula VI:

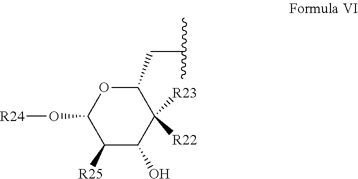

wherein R22 and R23 are selected from the group consisting of OH, F, and H with the proviso that if one of R22 or R23 is OH, the other is H; wherein R25 is OH or F; and wherein R24 is a C1-10 (hetero)alkyl or a compound according to Formula VII:

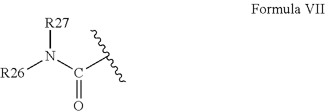

wherein R26 is H or a C1-C10 alkyl; and wherein R27 is a C1-C10 alkyl.

In another aspect, the present invention relates to a reagent compound according to Formula VIII:

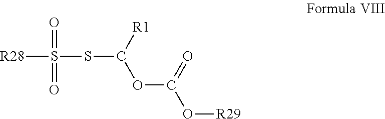

wherein R28 is methyl or 4-tolyl; wherein R1 is selected from the group consisting of hydrogen, methyl, ethyl, n-propyl, isopropyl, n-butyl, sec-butyl, and t-butyl, preferably R1 is hydrogen or methyl; and wherein R29 is pentafluorophenyl or 4-nitrophenyl.

This reagent compound is a precursor and will form the linker according to the present compound. On the side where R28 is present in the reagent compound the organic group G will be coupled. On the side where R29 is present in the reagent compound the drug moiety DM will be coupled. The present inventors have invented this novel and inventive reagent compound for preparing the compounds according to the present invention.

In another aspect, the present invention relates to a method for preparing a reagent compound (also called reagent Protocol) according to Formula VIII, said method comprising the steps of:
i) reacting a 1-chloroalkyl chloroformate of formula ClC(═O)OCH(R1)Cl with pentafluorophenol when R29 is pentafluorophenyl or with 4-nitrophenol when R29 is 4-nitrophenyl to give the corresponding substituted phenyl chloromethyl carbonate;
ii) reacting the substituted phenyl chloromethyl carbonate obtained in step i) with sodium iodide to give a substituted phenyl iodomethyl carbonate; and
iii) reacting the substituted phenyl iodomethyl carbonate obtained in step ii) with an alkali methanethiosulfonate, preferably sodium methanethiosulfonate, when R28 is methyl or alkali p-toluenethiosulfonate, preferably potassium p-toluenethiosulfonate, when R28 is 4-tolyl to give the reagent compound of Formula VIII.

Reaction step i is carried out in a solvent or a mixture of solvents, preferably selected from the group consisting of dichloromethane, chloroform, and THF, most preferably dichloromethane. This reaction step i is preferably carried out at a temperature of between −10 and 30° C., such as between 0 and 10° C. In a specific embodiment it is carried out at 0° C. Reaction step i is preferably carried out under an inert atmosphere, e.g. in the absence of oxygen, preferably under an argon or nitrogen atmosphere. Reaction step i is carried out for a duration of 1 to 4 hours. In an embodiment, a base or proton scavenger is present during step i, preferably selected from the group consisting of pyridine, triethylamine, and N,N-diisopropylethylamine, most preferably pyridine.

Reaction step ii is carried out in acetone. This reaction step ii is preferably carried out at a temperature of between 35 and 50° C. Reaction step ii is preferably carried out under an inert atmosphere, e.g. in the absence of oxygen, preferably under an argon or nitrogen atmosphere. Reaction step ii is carried out for a duration of 18 to 36 hours. In an embodiment, a base is present during step ii, preferably NaHCO₃ or KHCO₃, most preferably NaHCO₃.

Reaction step iii is carried out in a solvent or a mixture of solvents, preferably selected from the group consisting of DMF, DME, and NMP, most preferably DMF. This reaction step iii is preferably carried out at a temperature of between 15 and 30° C. In a specific embodiment it is carried out at 20° C. Reaction step iii is preferably carried out under an inert atmosphere, e.g. in the absence of oxygen, preferably under an argon or nitrogen atmosphere. Reaction step iii is carried out for a duration of 30 to 60 minutes.

The present inventors have invented this novel and inventive method for preparing the reagent compound that in turn may be used for preparing the compounds according to the present invention.

The present invention is related to methods for preparing the compounds according to the present invention. In one aspect (also called first protocol) the invention relates to a method for preparing a compound according to Formula I, said method comprising the steps of:

A) contacting a drug molecule [ZH]DM, ZH represents a part of DM; wherein ZH is selected from the group consisting of an alcohol, phenol, oxime, a primary amine, secondary amine, and a thiol, with the proviso that NH and NH₂ are not part of an amide, carbamate or urethane with a 1-chloroalkyl chloroformate of formula ClC(═O)OCH(R1)Cl to obtain an intermediate compound according to Formula X

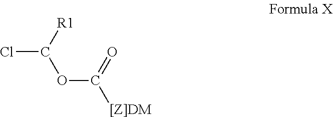

wherein R1 is selected from the group consisting of hydrogen, methyl, ethyl, n-propyl, isopropyl, n-butyl, sec-butyl, and t-butyl, preferably R1 is hydrogen or methyl;

B) contacting said intermediate compound according to Formula X obtained in step A) with an alkali methanethiosulfonate when R28 is methyl or with an alkali p-toluenethiosulfonate when R28 is 4-tolyl in order to obtain an intermediate compound according to Formula XI:

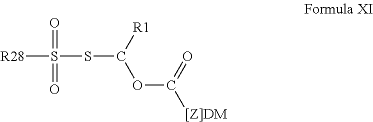

and C) reacting the intermediate compound of Formula XI obtained in step B) with G[C]—SH to provide the compound according to Formula I.

This specific method to prepare compounds according to the present invention according to the First Protocol (see claim 12) is schematically shown below.

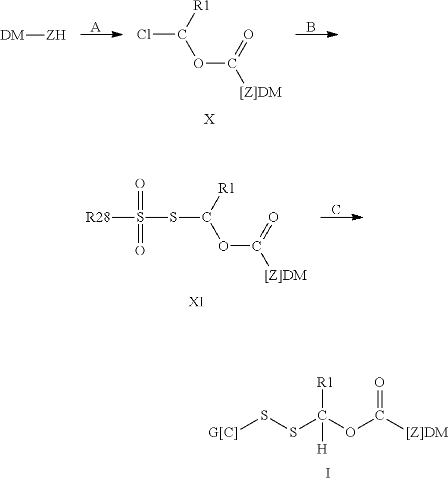

Reaction step A) is carried out in a solvent or a mixture of solvents, preferably selected from the group consisting of dichloromethane, chloroform, and THF, most preferably dichloromethane. This reaction step A) is preferably carried out at a temperature of between −10 and 30° C., such as between 0 and 10° C. In a specific embodiment it is carried out at 0° C. Reaction step A) is preferably carried out under an inert atmosphere, e.g. in the absence of oxygen, preferably under an argon or nitrogen atmosphere. Reaction step A) is carried out for a duration of 1 to 2 hours. In an embodiment, a base or proton scavenger is present during step A), preferably selected from the group consisting of triethylamine, tributylamine, and N,N-diisopropylethylamine, most preferably N,N-diisopropylethylamine.

Reaction step B) is carried out in a solvent or a mixture of solvents, preferably selected from the group consisting of methanol, ethanol, and DMF, most preferably ethanol. This reaction step B) is preferably carried out at a temperature of between 15 and 70° C. In a specific embodiment it is carried out at 70° C. Reaction step B) is preferably carried out under an inert atmosphere, e.g. in the absence of oxygen, preferably under an argon or nitrogen atmosphere. Reaction step B) is carried out for a duration of 1 to 24 hours.

Reaction step C) is carried out in a solvent or a mixture of solvents, preferably selected from the group consisting of methanol, ethanol, THF, and DMF, most preferably methanol. This reaction step C) is preferably carried out at a temperature of between 15 and 30° C. In a specific embodiment it is carried out at 20° C. Reaction step C) is preferably carried out under an inert atmosphere, e.g. in the absence of oxygen, preferably under an argon or nitrogen atmosphere. Reaction step C) is carried out for a duration of 5 min to 24 hours. In an embodiment, a base is present during step C), preferably NaHCO₃ or KHCO₃, most preferably NaHCO₃.

In another aspect, (also called second protocol) the present invention relates to a method for preparing a compound according to Formula Ia, said method comprises the steps of:
a) providing a reagent compound according to Formula VIII;
b) contacting the reagent compound provided in step a) with a drug molecule [NH]DM, NH represents a part of DM; with the proviso that NH is not part of an amide, carbamate or urethane to prepare an intermediate compound according to Formula IX:

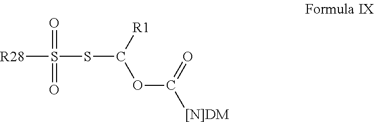

and c) reacting the intermediate compound of Formula IX obtained in step b) with G[C]—SH to provide the compound according to Formula Ia.

This specific method to prepare compounds according to the present invention according to the Second Protocol (see claim 11) is schematically shown below.

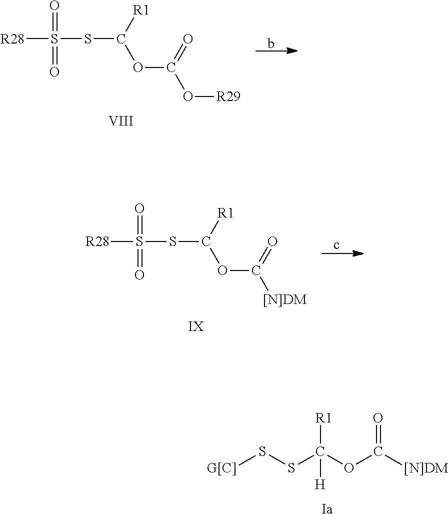

### Synthesis of Compound Ia According to Second Protocol

Reaction step b) is carried out in a solvent or a mixture of solvents, preferably selected from the group consisting of dichloromethane, methanol, THF, and DMF, most preferably dichloromethane. This reaction step b) is preferably carried out at a temperature of between 15 and 35° C. In a specific embodiment it is carried out at 20° C. Reaction step b) is preferably carried out under an inert atmosphere, e.g. in the absence of oxygen, preferably under an argon or nitrogen atmosphere. Reaction step b) is carried out for a duration of 1 to 24 hours. In an embodiment, a base is present during step b), preferably selected from the group consisting of triethylamine, tributylamine, and N,N-diisopropylethylamine, most preferably triethylamine. In case amine drugs are found during the procedure to be less reactive—in other words, the reaction is too slow—the reaction can be accelerated by adding 1-hydroxybenzotriazole (preferably one equivalent).

Reaction step c) is carried out in a solvent or a mixture of solvents, preferably selected from the group consisting of methanol, THF, and DMF, preferably methanol. This reaction step c) is preferably carried out at a temperature of between 15 and 30° C. In a specific embodiment it is carried out at 20° C. Reaction step c) is preferably carried out under an inert atmosphere, e.g. in the absence of oxygen, preferably under an argon or nitrogen atmosphere. Reaction step c) is carried out for a duration of 5 min to 24 hours. In an embodiment, a base is present during step c), preferably selected from the group consisting of NaHCO₃ or KHCO₃, most preferably NaHCO₃.

### Optional Step of Removal of Protective Groups in G[C] Moiety

### First Protocol—Step D or Second Protocol—Step d

In both the first and second protocol an optional step in the synthesis of a compound according to Formula I or Ia may be present. This optional step (called step D or d) can be present as an optional step in claim 12 or 11 respectively, after step C or c, respectively.

In this embodiment steps c) and d) of the second protocol (claim 11) are as follows:
c) reacting the intermediate compound of Formula IX obtained in step b) with G[C]—SH, wherein the hydroxyl groups of G[C]—SH are protected by protective groups, to provide a compound according to Formula Ia in which protective groups are present in the hydroxyl groups of the G[C] moiety; and
d) removal of the protective groups present on the hydroxyl groups in the G[C] moiety to provide the compound according to Formula Ia.

In this embodiment steps C) and D) of the first protocol (claim 12) are as follows:
C) reacting the intermediate compound of Formula XI obtained in step B) with G[C]—SH, wherein the hydroxyl groups of G[C]—SH are protected by protective groups, to provide a compound according to Formula I in which protective groups are present on the hydroxyl groups of the G[C] moiety; and
D) removal of the protective groups present on the hydroxyl groups in the G[C] moiety to provide the compound according to Formula I.

### Optional Step of Removal of Protective Groups in Drug Moiety

### First Protocol—Step E or Second Protocol—Step e

In both the first and second protocol an optional step in the synthesis of a compound according to Formula I or Ia may be present. The step E can be present as an optional step in claim 12, after step C (in case there are no protective groups on the G(C) moiety) or after step D and the step e can be present as an optional step in claim 11, after step c (in case there are no protective groups on the G(C) moiety) or after step d.

In this embodiment steps a), b), c) and e) of the second protocol (claim 11) are as follows:
a) providing a reagent compound according to Formula VIII;
b) contacting the reagent compound provided in step a) with a drug molecule [NH]DM, having at least one protective group on a hydroxy, primary or secondary amine, indole, imidazole, triazole, tetrazole, amidine, thiol, carboxylate, phosphate, phosphonate, sulfate or sulfonate; NH represents a part of DM; with the proviso that NH is not part of an amide, carbamate or urethane to prepare an intermediate compound according to Formula IX in which at least one protective group is present on the hydroxy, primary or secondary amine, indole, imidazole, triazole, tetrazole, amidine, thiol, carboxylate, phosphate, phosphonate, sulfate or sulfonate of the DM moiety;
c) reacting the intermediate compound of Formula IX obtained in step b) with G[C]—SH to provide a compound according to Formula Ia in which at least one protective group is present on the hydroxy, primary or secondary amine, indole, imidazole, triazole, tetrazole, amidine, thiol, carboxylate, phosphate, phosphonate, sulfate or sulfonate of the DM moiety; and
e) removal of the at least one protective group present on the hydroxy, primary or secondary amine, indole, imidazole, triazole, tetrazole, amidine, thiol, carboxylate, phosphate, phosphonate, sulfate or sulfonate of the DM moiety to provide the compound according to Formula Ia.

In this embodiment steps A), B), C) and D) of the first protocol (claim 12) are as follows:
A) contacting a drug molecule [ZH]DM, having at least one protective group on a hydroxy, primary or secondary amine, indole, imidazole, triazole, tetrazole, amidine, thiol, carboxylate, phosphate, phosphonate, sulfate or sulfonate of the DM moiety, [ZH] represents a part of DM; wherein ZH is selected from the group consisting of an alcohol, phenol, oxime, a primary amine, secondary amine, and a thiol, with the proviso that NH and NH2 are not part of an amide, carbamate or urethane with a 1-chloroalkyl chloroformate of formula ClC(═O)OCH(R1)Cl to obtain an intermediate compound according to Formula X in which at least one protective group is present on the hydroxy, primary or secondary amine, indole, imidazole, triazole, tetrazole, amidine, thiol, carboxylate, phosphate, phosphonate, sulfate or sulfonate of the DM moiety;
B) contacting said intermediate compound according to Formula X obtained in step A) with an alkali methanethiosulfonate when R28 is methyl or with an alkali p-toluenethiosulfonate when R28 is 4-tolyl in order to obtain an intermediate compound according to Formula XI in which at least one protective group is present on the hydroxy, primary or secondary amine, indole, imidazole, triazole, tetrazole, amidine, thiol, carboxylate, phosphate, phosphonate, sulfate or sulfonate of the DM moiety;
C) reacting the intermediate compound of Formula XI obtained in step B) with G[C]—SH to provide a compound according to Formula I in which at least one protective group is present on the hydroxy, primary or secondary amine, indole, imidazole, triazole, tetrazole, amidine, thiol, carboxylate, phosphate, phosphonate, sulfate or sulfonate of the DM moiety; and
D) removal of the protective groups present on the hydroxy, primary or secondary amine, indole, imidazole, triazole, tetrazole, amidine, thiol, carboxylate, phosphate, phosphonate, sulfate or sulfonate of the DM moiety to provide the compound according to Formula I.

In an embodiment of said methods for preparing the present compound, the drug molecule [ZH]DM is selected from the group consisting of from 5′-Deoxy-5-fluorocytidine, Cytarabine, Lenalidomide, Thalidomide, Acyclovir, Doxorubicin, Losartan, Ciclopirox, Albendazole, Duloxetine, Mesalazine, Linagliptin, Atomoxetine, 5-Fluorouracil, Methylphenidate, Palbociclib, Azacitidine, Gabapentin, Metoprolol, Nintedanib, Carvedilol, Gemcitabine, Rasagiline, Pscilocin, Celecoxib, Ibrutinib, Riluzole, Meropenem, Cinacalcet, Lapatinib, Tamiflu, Ceftriaxon, Abiraterone, Fesoterodine, Rotigotine, Orciprenaline, Acyclovir, Fulvestrant, Tenofovir, Ganciclovir, Testosterone, Kalydeco, Tizoxanide, Cannabidiol, Paliperidone, Venlafaxine, Edaravone, Paracetamol, Vorinostat, gemcitabine, Paclitaxel, Estradiol, 17-Ethynyl-estradiol, Propofol, Mercaptopurin, Acetylcysteine, Bucillamine, Captopril, and Zofenoprilat.

In an embodiment of the present compound, the compound is a prodrug comprising of a promoiety G, coupled via a linker to a drug moiety, preferably wherein drug moiety is obtained from a drug molecule selected from the group consisting of from 5′-Deoxy-5-fluorocytidine, Cytarabine, Lenalidomide, Thalidomide, Acyclovir, Doxorubicin, Losartan, Ciclopirox, Albendazole, Duloxetine, Mesalazine, Linagliptin, Atomoxetine, 5-Fluorouracil, Methylphenidate, Palbociclib, Azacitidine, Gabapentin, Metoprolol, Nintedanib, Carvedilol, Gemcitabine, Rasagiline, Pscilocin, Celecoxib, Ibrutinib, Riluzole, Meropenem, Cinacalcet, Lapatinib, Tamiflu, Ceftriaxon, Abiraterone, Fesoterodine, Rotigotine, Orciprenaline, Acyclovir, Fulvestrant, Tenofovir, Ganciclovir, Testosterone, Kalydeco, Tizoxanide, Cannabidiol, Paliperidone, Venlafaxine, Edaravone, Paracetamol, Vorinostat, gemcitabine, Paclitaxel, Estradiol, 17-Ethynyl-estradiol, Propofol, Mercaptopurin, Acetylcysteine, Bucillamine, Captopril, and Zofenoprilat.

The present invention has as one of its aims to increase the oral bioavailability of drugs. Oral bioavailability is usually assessed by determining the area under the plasma concentration-time curve (AUC) [see publication ADMET for medicinal chemists, Tsaioun, K and Kates, S. A. (Eds.), 2011, Ch. 5, Wiley]. Plasma drug concentration increases with extent of absorption, the peak concentration is reached when drug elimination rate equals absorption rate. Peak time is the most widely used general index of absorption rate; the slower the absorption, the later the peak time. The most reliable measure of a drug's oral bioavailability is AUC. The AUC is directly proportional to the total amount of unchanged drug that reaches systemic circulation. Drug products may be considered bioequivalent in extent and rate of absorption if their plasma concentration curves are essentially superimposable. In practical terms, the oral bioavailability is the percentage of the AUC of a drug available in the blood of a test species after oral administration in relation to the AUC obtained from the same dose administered intravenously to the test subject. A broad spectrum of methods is available for determining intestinal absorption of compounds in experimental animals. Typical laboratory methods include perfusion via (multiple) lumen tubes, mass balance studies and blood kinetics following oral and intravenous administration of the compound [see http://www.rivm.nl/bibliotheek/rapporten/630030001.pdf]. Relevant animal species include mice, rats, dogs, mini pigs and monkey. Oral bioavailability of a drug and its conjugate can also be predicted to some extent using appropriate in vitro models [see publication Altern. Lab. Anim., 2001, 29, 649-668]. Appropriate in vitro tissue models include everted gut sac, perfused intestinal segments and Ussing chambers. Cell-based in vitro models include small-intestinal cell lines from fetal and neonatal rats and Caco-2 cells.

The term “increasing the oral bioavailability of a drug” or “increased bioavailability” is used herein to indicate that the oral bioavailability of a drug modified according to the invention is increased in comparison to the unmodified drug (being DM-ZH]. Even a small increase of oral bioavailability can be relevant. For instance, if the drug currently has an oral bioavailability of 10%, an increase to 11 or 12% using the compounds according to the invention is considered a relevant increase. For example, a drug currently having an oral bioavailability of 10% may form a prodrug of the invention which, upon oral administration, leads to the accumulation of the unconjugated drug with an oral bioavailability of more than 10%. The increase in oral bioavailability may be in the order of a few percent points, resulting in an increased bioavailability of 11%, 12%, 13%, 14% or 15% or even more, such as up to 20%. More spectacular increases have also been observed; depending on the drug and type of prodrug, oral bioavailabilities of up to 30%, 40% or 50% or even more such as 60% up to 70% appeared achievable. In certain cases, the increase was even more, such as 71 up to 90%. In exceptional cases, even higher oral bioavailability may be achieved such as 91% up to even 100%.

In an embodiment, the present invention relates to the use of the compound as a linker attached to a drug molecule to improve the oral bioavailability by at least 1%, preferably by at least 2% compared to the oral bioavailability of the drug molecule as such. With an increase of a certain % an incremental increase is meant. When the oral bioavailability of a certain drug is 10% and after it has been prepared in a compound according to the invention the oral bioavailability of said drug is 11%, this is considered an increase of 1%.

The increase of oral bioavailability achieved by the method according to the invention may depend on the drug and type of prodrug used. It has been observed that a drug conjugate as prepared using a method according to the invention leads to a higher concentration of the drug (i.e., without the conjugated promoiety) in circulation upon oral administration, compared to the concentration of the same unconjugated drug when administered orally.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The scope of the present invention is defined by the appended claims. One or more of the objects of the invention are achieved by the appended claims.

### EXAMPLES

The present invention is further elucidated based on the Examples below which are illustrative only and not considered limiting to the present invention.

### Example not According to the Invention—Cinacalcet

The present inventors have tested the usefulness of these known 2-disulfanylethyl carbamate linker for increasing the oral bioavailability of the drug Cinacalcet:

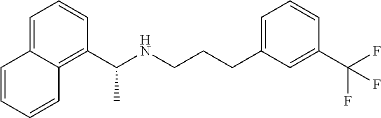 [SMILES: C[C@@H](NCCCc1cccc(C(F)(F)F)c1)c1cccc2ccccc12]

In order to do so, a 2-disulfanylethyl carbamate 102 analogue having a sugar promoiety of Cinacalcet was synthesized.

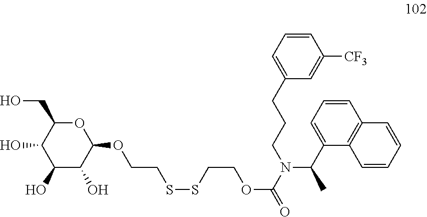 [SMILES: C[C@H](c1cccc2ccccc12)N(CCCc1cccc(C(F)(F)F)c1)C(=O)OCCSSCCO[C@@H]1O[C@H](CO)[C@@H](O)[C@H](O)[C@H]1O]

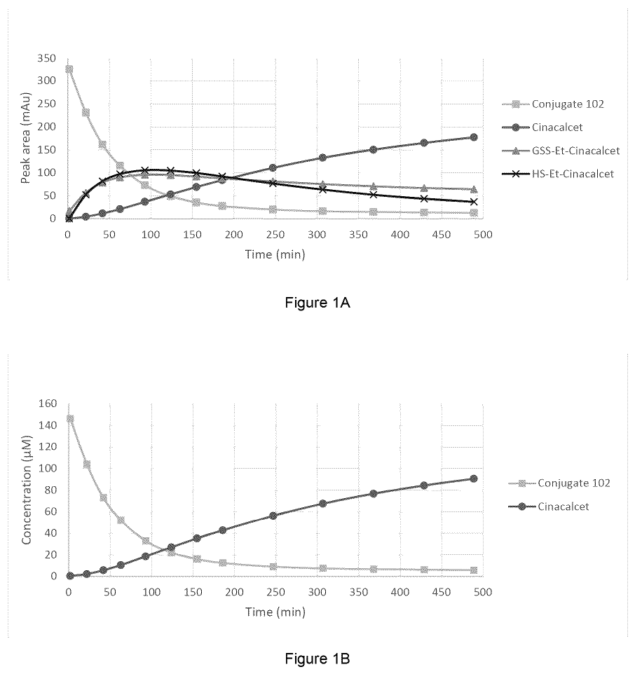

In vitro treatment of 102 with glutathione (5 eq., phosphate buffer pH=7.6, 37° C.) resulted in a very slow and incomplete formation of Cinacalcet, together with significant amounts of the mercaptoethyl carbamate intermediate (HS-Et-Cinacalcet) and its glutathione adduct (GSS-Et-Cinacalcet), the concentration of both of which gradually diminished over time. The glutathione adduct was found to be somewhat more stable than the mercaptoethyl carbamate. The results are shown in FIGS. 1A and B. In FIG. 1A is shown the disappearance of conjugate 102 over time after treatment with glutathione, the formation of Cinacalcet and the appearance and partial disappearance of an intermediate (HS-Et-Cinacalcet) and a glutathione adduct (GSS—Et-Cinacalcet)—see below.

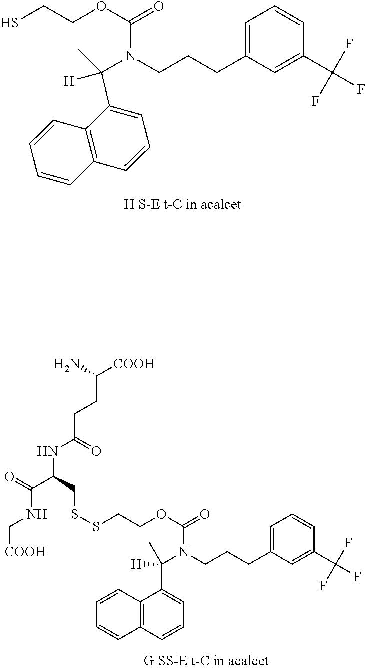 [SMILES: CC(c1cccc2ccccc12)N(CCCc1cccc(C(F)(F)F)c1)C(=O)OCCS ; C[C@H](c1cccc2ccccc12)N(CCCc1cccc(C(F)(F)F)c1)C(=O)OCCSSC[C@H](NC(=O)CC[C@H](N)C(=O)O)C(=O)NCC(=O)O]

HS-Et-Cinacalcet refers to Cinacalcet with a 2-disulfanylethyl carbamate linker and GSS-Et-Cinacalcet refers to glutathione coupled to cinacalcet 2-disulfanylethyl carbamate via a disulfide bond. Moreover, the in vitro treatment of 102 with glutathione shows that the pro-drugs is far from being completely converted into Cinacalcet within 8 hours. The results are shown in FIG. 1B. In FIG. 1B is shown the disappearance of conjugate 102 and the formation of Cinacalcet. In this figure conjugate 102 refers to the compound disclosed above. Apparently, the 2-mercaptoethyl carbamate does not easily degrade itself. Oral administration of 102 to beagle dogs did not produce any detectable amount of the drug Cinacalcet. The in vivo results suggest that the 2-mercaptoethylcarbamate-substituted drug and the corresponding glutathione adduct are relatively stable and also do not appear to be substrates for suitable hydrolytic enzyme that are able to convert the intermediates into Cinacalcet. Therefore, this linker is not very suitable for use in drug delivery. Moreover, the in vitro treatment of 102 with glutathione shows that less than half of the pro-drugs are converted into Cinacalcet in 23 hours.

### Example not According to the Invention—Duloxetine

The present inventors have tested the usefulness of these known 2-disulfanylethyl carbamate linker for increasing the oral bioavailability of the drug duloxetine:

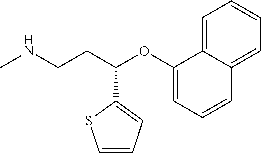 [SMILES: CNCC[C@H](Oc1cccc2ccccc12)c1cccs1]

In order to do so, a 2-disulfanylethyl carbamate 103 analogue having a sugar promoiety of duloxetine was synthesized:

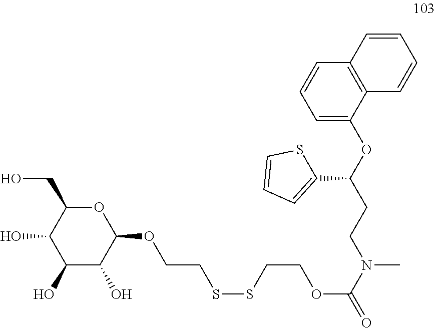 [SMILES: CN(CC[C@@H](Oc1cccc2ccccc12)c1cccs1)C(=O)OCCSSCCO[C@@H]1O[C@H](CO)[C@@H](O)[C@H](O)[C@H]1O]

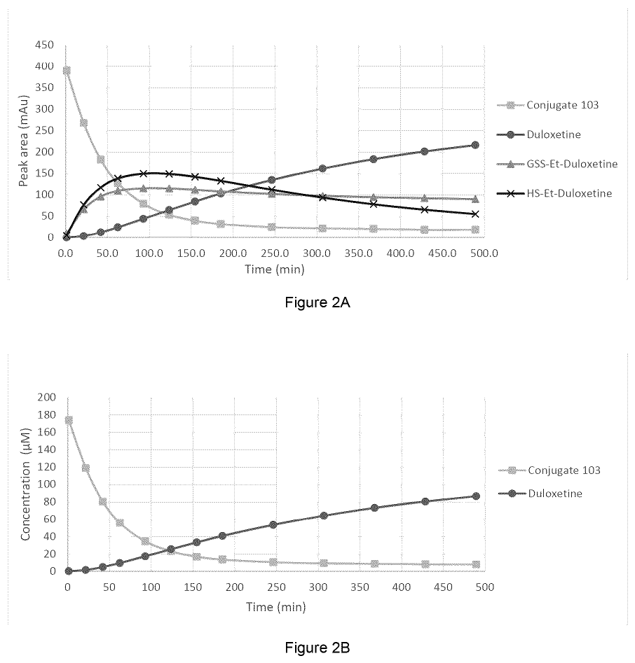

Treatment of duloxetine conjugate 103 with glutathione gave a similar outcome and also resulted in the very slow and incomplete formation of duloxetine while significant amounts of mercaptoethyl carbamate and the corresponding glutathione adduct were observed. The results are shown in FIGS. 2A and B. In FIG. 2A is shown the disappearance of conjugate 103, the formation of Duloxetine and the appearance and partial disappearance of an intermediate (HS-Et-Duloxetine) and a glutathione adduct (GSS-Et-Duloxetine). In FIG. 2B is shown the disappearance of 103 and the formation of Duloxetine. In this figure conjugate 103 refers to the compound disclosed above.

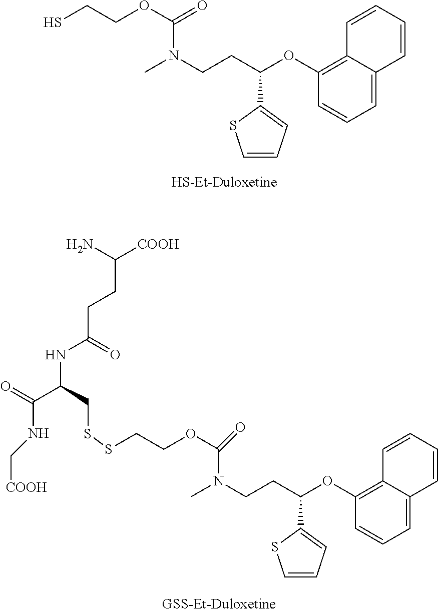 [SMILES: CN(CC[C@H](Oc1cccc2ccccc12)c1cccs1)C(=O)OCCS ; CN(CC[C@H](Oc1cccc2ccccc12)c1cccs1)C(=O)OCCSSC[C@H](NC(=O)CCC(N)C(=O)O)C(=O)NCC(=O)O]

HS-Et-Duloxetine refers to Duloxetine 2-disulfanylethyl carbamate and GSS-Et-Duloxetine refers to glutathione coupled to Duloxetine 2-disulfanylethyl carbamate via a disulfide bond. Moreover, the in vitro treatment of 103 with glutathione shows that less than half of the pro-drugs is converted into Duloxetine within 8 hours. The results are shown in FIG. 2B. Oral administration of 103 to beagle dogs also did not produce any detectable amount of Duloxetine. Therefore, this linker is not very suitable for use in drug delivery.

### Example According to the Invention—Cinacalcet

The present inventors have tested the usefulness of the inventive compounds for increasing the oral bioavailability of the drug Cinacalcet. In order to do so, a 2-disulfanylmethyl carbamate 60 analogue having a sugar promoiety of Cinacalcet was synthesized:

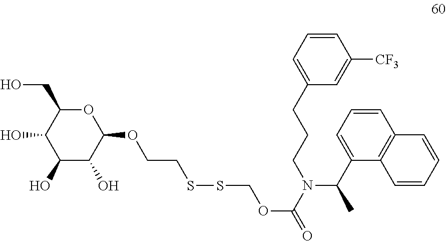 [SMILES: C[C@H](c1cccc2ccccc12)N(CCCc1cccc(C(F)(F)F)c1)C(=O)OCSSCCO[C@@H]1O[C@H](CO)[C@@H](O)[C@H](O)[C@H]1O]

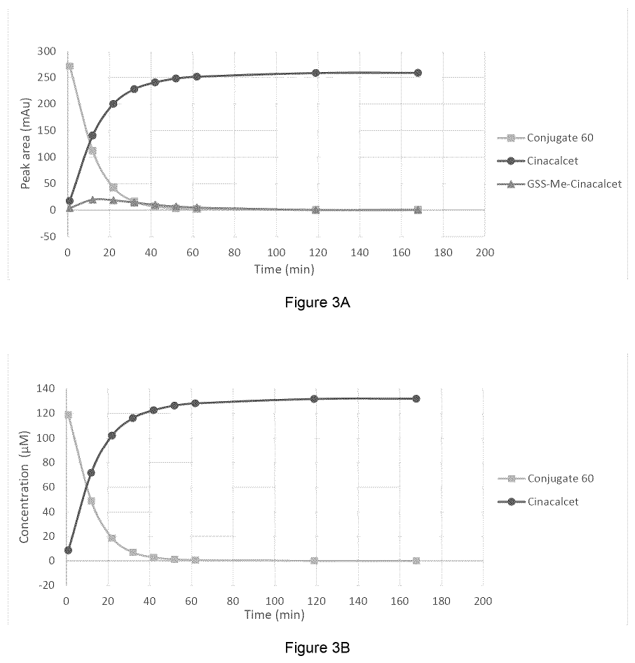

In vitro treatment of 60 with glutathione resulted in the very fast and complete formation of Cinacalcet. A trace of a glutathione adduct was also observed but was rapidly converted into Cinacalcet. Interestingly, the degradation of the glutathione adduct formed from 60 proceeds much faster than the degradation of the glutathione adduct from 102, which can be acknowledged as an additional advantage. FIG. 3A shows the graph depicting this. In FIG. 3A is shown the disappearance of conjugate 60, the formation of Cinacalcet and the appearance and disappearance of the glutathione adduct (GSS-Me-Cinacalcet)—see below.

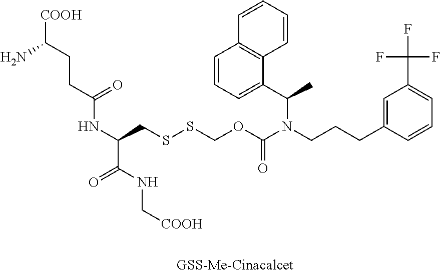 [SMILES: C[C@H](c1cccc2ccccc12)N(CCCc1cccc(C(F)(F)F)c1)C(=O)OCSSC[C@H](NC(=O)CC[C@H](N)C(=O)O)C(=O)NCC(=O)O]

GSS-Me-Cinacalcet refers to glutathione coupled to Cinacalcet disulfanylmethyl carbamate via a disulfide bond. The in vitro treatment of 60 with glutathione shows that this pro-drug is rapidly and completely converted into the desired drug as shown in FIG. 3B. In FIG. 3B is shown the disappearance of 60 and the formation of Cinacalcet. The results represent the concentration of 60 and Cinacalcet versus time. Indeed, over 90% conversion into the parent drug is observed within 1 h of treatment. A small amount of the glutathione adduct GSS-Me-Cinacalcet was observed, which completely converted into Cinacalcet within one hour. No thiomethycarbamate intermediate of Cinacalcet was observed. In vivo pharmacokinetic studies have learned that substituted 1-(disulfanyl)alkyloxycarbonyl linker-type prodrugs are readily converted into the parent drug. Thus, oral administration of 60, which is the methylene variant of cinacalcet conjugate 102, to beagle dogs readily resulted in the generation of significant amounts of cinacalcet. Without wanting to be bound by theory, cleavage of the S—S bond is expected to result in the formation of an unstable 1-sulfanylalkyl carbamate or carbonate intermediate, which decompose readily to produce the active drug. These features have significant advantages over the previously mentioned 2-disulfanylethyl carbamates.

### Example According to the Invention—Duloxetine

The present inventors have tested the usefulness of the inventive compounds for increasing the oral bioavailability of the drug Duloxetine. In order to do so, a disulfanylmethyl carbamate 61 analogue having a sugar promoiety of Duloxetine was synthesized.

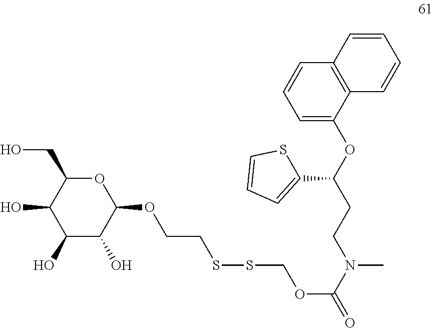 [SMILES: CN(CC[C@@H](Oc1cccc2ccccc12)c1cccs1)C(=O)OCSSCCO[C@@H]1O[C@H](CO)[C@H](O)[C@H](O)[C@H]1O]

In vitro treatment of 61 with glutathione resulted in the very fast and complete formation of Duloxetine. A trace of a glutathione adduct GSS-Me-Duloxetine was also observed but was rapidly converted into Duloxetine. Interestingly, the degradation of the glutathione adduct formed from 61 proceeds much faster than the degradation of the glutathione adduct from 103, which can be acknowledged as an additional advantage.

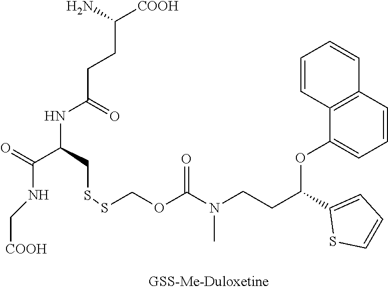 [SMILES: CN(CC[C@H](Oc1cccc2ccccc12)c1cccs1)C(=O)OCSSC[C@H](NC(=O)CC[C@H](N)C(=O)O)C(=O)NCC(=O)O]

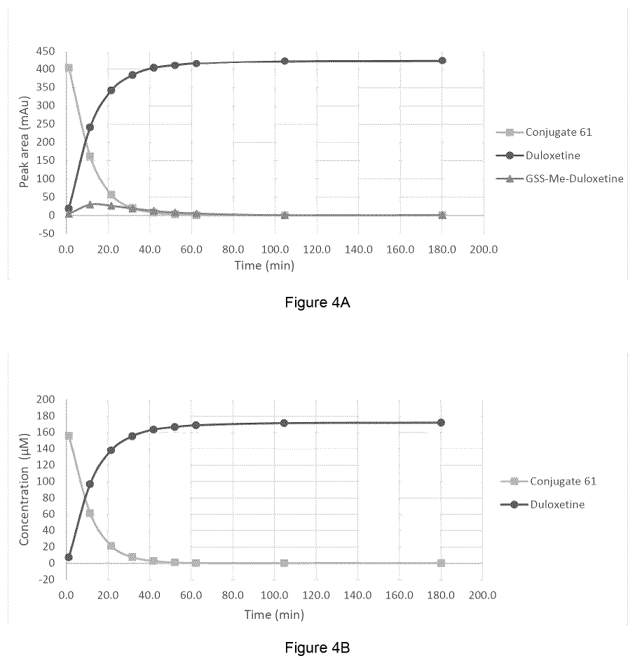

In FIG. 4A GSS-Me-Duloxetine refers to Glutathione coupled to Duloxetine disulfanylmethyl carbamate via a disulfide bond. FIGS. 4A and 4B are graphs of glutathione treatment of Duloxetine conjugate 61 according to the invention. In FIG. 4A is shown the disappearance of 61, the formation of Duloxetine and the appearance and disappearance of the glutathione adduct (GSS-Me-Duloxetine)—see above. In FIG. 4B is shown the disappearance of 61 and the formation of Duloxetine. The results represent the concentration of 61 and Duloxetine versus time. The in vitro treatment of 61 with glutathione shows that this pro-drug is rapidly and completely converted into the desired drug as shown in FIG. 4B. Indeed, over 90% conversion into the parent drug is observed within 1 h of treatment. As also observed for the Cinacalcet conjugate 60 (see example above), a small amount of the glutathione adduct GSS-Me-Duloxetine was observed, which completely converted into Duloxetine within one hour. No thiomethylcarbamate intermediate of Duloxetine was observed.

In vivo pharmacokinetic studies have learned that substituted 1-(disulfanyl)alkyloxycarbonyl linker-type prodrugs are readily converted into the parent drug. Thus, oral administration of 61, which is the methylene variant of duloxetine conjugate 103, to beagle dogs readily resulted in the generation of significant amounts of duloxetine. Without wanting to be bound by theory, cleavage of the S—S bond is expected to result in the formation of an unstable 1-sulfanylalky carbamate or carbonate intermediate, which decompose readily to produce the active drug. These features have significant advantages over the previously mentioned 2-disulfanylethyl carbamates.

### Example According to the Invention—Determination of Oral Bioavailability of Several Conjugates

Relative and absolute bioavailability may be determined in different animal models and according to different protocols. The following protocol is typical for determining bioavailability in female Beagle dogs and was used in the present invention. The animals were deprived from food over a time period of 8 h prior to administration of the compounds according to the present invention and over a time period of 2 h after administration of the compounds according to the present invention. Water was supplied without limitation. On the study day, the animals received the compounds according to the present invention, at a single dose of 7.5 or 15 μmol/kg, by oral gavage, formulated in mixtures of propylene glycol, ethanol and 0.9% NaCl+5% mannitol in water. Blood samples were collected from the jugular vein on the following time points: 0.25, 0.5, 1, 2, 4, 8 and 24 hours after dosing of the compounds according to the present invention. Circulating concentrations of the compounds according to the present invention were determined over a time period of 24 hours using LC-MS/MS methods with demonstrated specificity and error over a concentration range of 1.0 ng/mL (LLQ) to 2500 ng/mL (1 day validation). Pharmacokinetic parameters were calculated from concentration versus time data using non-compartmental pharmacokinetic methods using Phoenix pharmacokinetic software. Data are compared to the parent drugs to establish improvement of its oral bioavailability by the compounds according to the present invention. The following compounds have been tested:

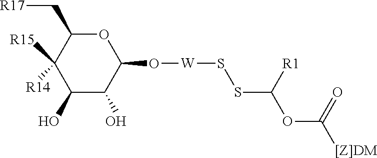

Table 1a shows the result. In the final column AAUC refers to the increase of AUC values for parent drugs derived from their conjugates after administration to Beagle dogs compared to AUC values obtained for the parent drugs as such after administration to Beagle dogs. This shows the effect of the use of the compounds according to the present invention. In this column: + denotes a 1.1 to 2-fold increase of the AUC compared to parent drug; ++ denotes a 2 to 4-fold increase of the AUC compared to the parent drug; and +++ denotes a >4-fold increase of the AUC compared to the parent drug.

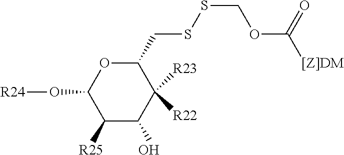

Table 1b shows the result. In the final column AAUC refers to the increase of AUC values for parent drugs derived from their conjugates after administration to Beagle dogs compared to AUC values obtained for the parent drugs as such after administration to Beagle dogs. This shows the effect of the use of the compounds according to the present invention. In this column: + denotes a 1.1 to 2-fold increase of the AUC compared to parent drug; ++ denotes a 2 to 4-fold increase of the AUC compared to the parent drug; and +++ denotes a >4-fold increase of the AUC compared to the parent drug.

TABLE 1a
Results for oral bioavailability for compounds according to the invention

| | | | | | | Drug | |
|---|---|---|---|---|---|---|---|
| Comp. | R14 | R15 | R17 | W | R1 | molecule used | Δ AUC |
| 60 | OH | H | OH | CH₂CH₂ | H | Cinacalcet | ++ |
| 62 | H | OH | OH | CH₂CH₂ | H | Abiraterone | ++ |
| 59 | H | OH | OH | CH₂CH₂ | H | Cinacalcet | +++ |
| 61 | H | OH | OH | CH₂CH₂ | H | Duloxetine | ++ |
| 66 | H | OH | OH | CH₂CH₂ | H | Ritalin | ++ |
| 70 | H | OH | OH | CH₂CH(CH₃) | H | Duloxetine | ++ |
| 74 | H | OH | OH | CH₂CH₂CH₂ | H | Cinacalcet | + |
| 75 | H | OH | OH | CH₂CH₂CH₂ | H | Duloxetine | ++ |
| 69 | H | OH | OH | CH(CH₃)CH₂CH₂ | H | Cinacalcet | +++ |
| 68 | H | OH | OH | CH₂CH₂ | Me | Cinacalcet | + |
| 65 | H | OH | OH | CH₂CH₂ | H | Rasagiline | ++ |
| 90 | OH | H | OH | C(O)NHCH₂CH₂ | H | Cinacalcet | + |
| 71 | H | OH | OH | C(O)N(methyl)-CH₂CH₂ | H | Abiraterone | +++ |
| 72 | H | OH | OH | C(O)N(methyl)-CH₂CH₂ | H | Cinacalcet | ++ |
| 73 | H | OH | OH | C(O)N(methyl)-CH₂CH₂ | H | Duloxetine | + |
| 78 | H | OH | OH | C(O)-1-(NC₃H₅)-3- | H | Duloxetine | ++ |
| 77 | H | OH | OH | C(O)-1-(NC₅H₉)-4- | H | Cinacalcet | ++ |
| 80 | OH | H | OH | C(O)N(propyl)CH₂ | H | Duloxetine | +++ |
| 79 | H | OH | OH | C(O)N(methyl)CH₂ | H | Duloxetine | ++ |
| 92a | OH | H | OH | C(O)N(propyl)CH₂OCH₂CH₂ | H | Cinacalcet | +++ |
| 92b | OH | H | F | C(O)N(propyl)CH₂OCH₂CH₂ | H | Cinacalcet | +++ |
| 93a | H | OH | OH | C(O)N(propyl)CH₂OCH₂CH₂ | H | Cinacalcet | +++ |
| 82 | H | OH | OH | C(O)N(propyl)CH₂OCH₂CH₂ | H | Duloxetine | +++ |
| 83 | H | OH | OH | C(O)N(CH₂CH₂OMe)CH₂O—CH₂CH₂ | H | Duloxetine | +++ |

TABLE 1b
Results for oral bioavailability for compounds according to the invention

| | | | | | Drug | |
|---|---|---|---|---|---|---|
| Comp. | R22 | R23 | R24 | R25 | molecule used | Δ AUC |
| 85 | OH | H | C(O)NHC₃H₇ | OH | Cinacalcet | ++ |
| 86 | OH | H | C(O)N(Ethyl)₂ | OH | Cinacalcet | ++ |
| 87 | OH | H | C(O)N(Methyl)C₃H₇ | OH | Duloxetine | +++ |
| 88 | H | OH | C(O)NHC₃H₇ | OH | Abiraterone | +++ |
| 84 | H | OH | C(O)NHC₃H₇ | OH | Cinacalcet | +++ |

The above examples clearly show the effect of the compounds according to the present invention in increasing the oral bioavailability of drugs by attached to a disulfide type linker and promoiety.

### Synthesis Examples

LC-MS data were recorded on an Agilent 1200 Infinity UPLC system, attached to an Agilent 6100 single quadrupole MS detector. A Kinetex 2.6μ EVO C18 100A column of 50×2.1 mm equipped with a EVO C18 guard column (Phenomenex) was used. The LC-MS experiments were run at a flow speed of 0.6 mL/min with a weakly acidic solvent system consisting of 0.1% formic acid in water (A) and acetonitrile containing 0.1% formic acid (B) was used. A gradient was run from 5% B to 60% B in 1.0 minutes, followed by a gradient from 60% to 95% B in 2.0 minutes and keeping the gradient at 95% B for 1 to 6 minutes.

### Preparation of Intermediate Thiosulfonate-Drug Conjugates

### First Protocol—Steps A and B

The below synthesis is a part of the synthesis of a compound according to Formula I.

In this case Z was N. The steps A and B according to claim 12.

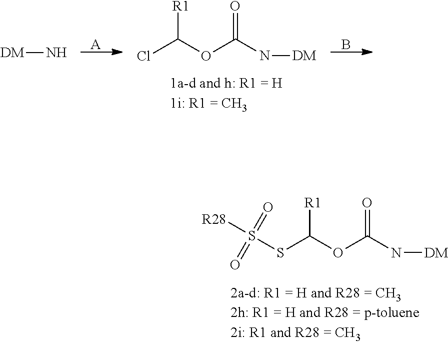

### Synthesis of Compounds 1

To a solution of amine containing drug (10 mmol) and DIPEA (23 mmol, 2.3 eq.) in DCM (65 mL) was added dropwise the appropriate 1-chloroalkyl chloroformate—depending on the R1 group selected—(13 mmol, 1.3 eq.) at 0° C. under an inert atmosphere of nitrogen. The reaction mixture was stirred at 0° C. under N₂. After completion, the reaction mixture was diluted with DCM, washed with water, a saturated solution of NaHCO₃ and brine and dried over MgSO₄. The crude product was concentrated and purified by column chromatography to yield a compound 1 (according to Formula X). This product was used in the next step to yield a compound 2 (According to Formula XI). Several drugs have been tested; Tables 2a below shows the compounds 2 that have been prepared.

### Synthesis of Compounds 2

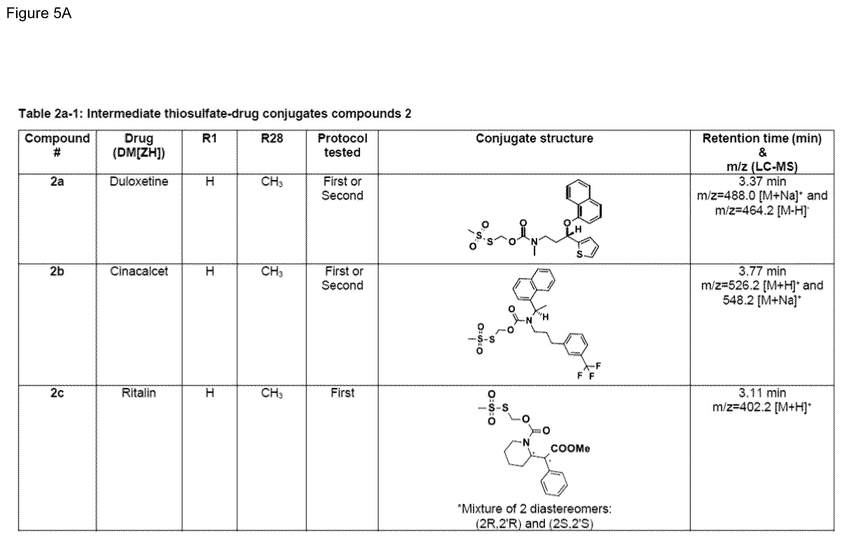

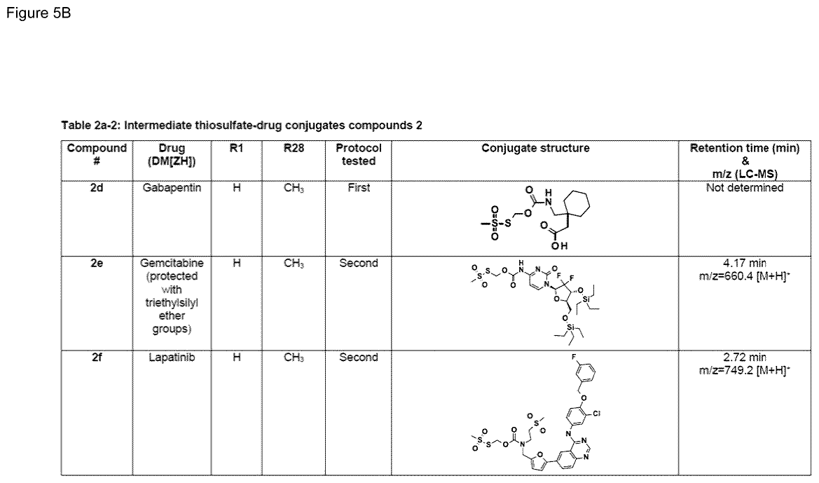

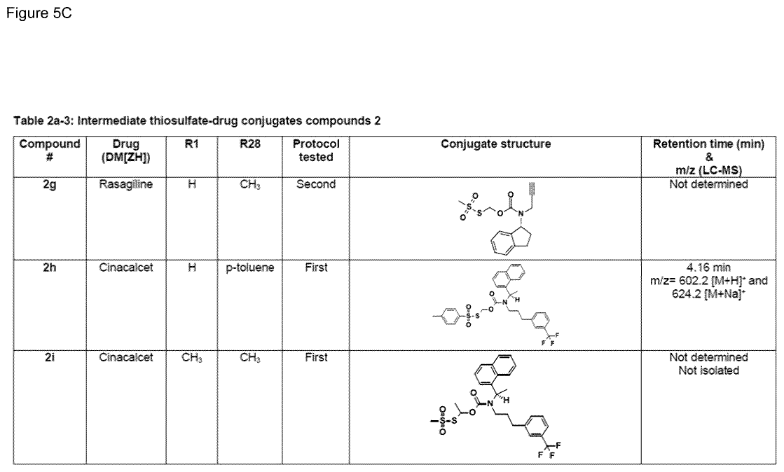

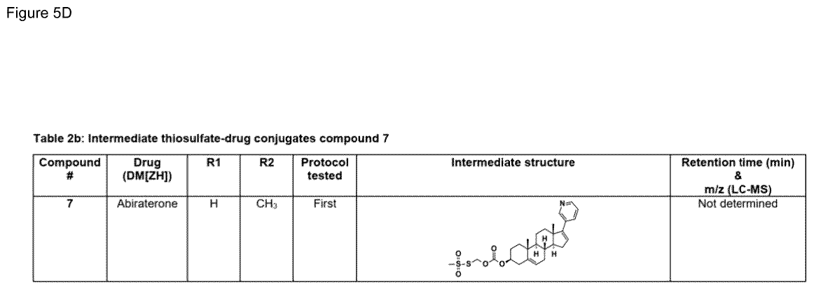

A solution of sodium methanethiosulfonate or potassium p-toluene thiosulfonate (6 mmol, 1.2 eq.) and compound 1 (5 mmol, 1 eq.) in EtOH (17 mL) was stirred at 70° C. After completion, the reaction mixture was concentrated and purified by column chromatography yielding 2 (according to Formula XI). Several drugs have been tested; Tables 2a in FIGS. 5A-B show the compounds 2 that have been prepared. Table 2a-1: compounds 2a-c/Table 2a-2: compounds 2d-f/Table 2a-3: compounds 2g-i.

### Reagent Protocol—Steps i, ii, and iii

The below synthesis is a synthesis of a reagent compound according to Formula VIII wherein R1 is hydrogen, R28 is methyl and R29 is pentafluorophenyl. This method is according to claim 10.

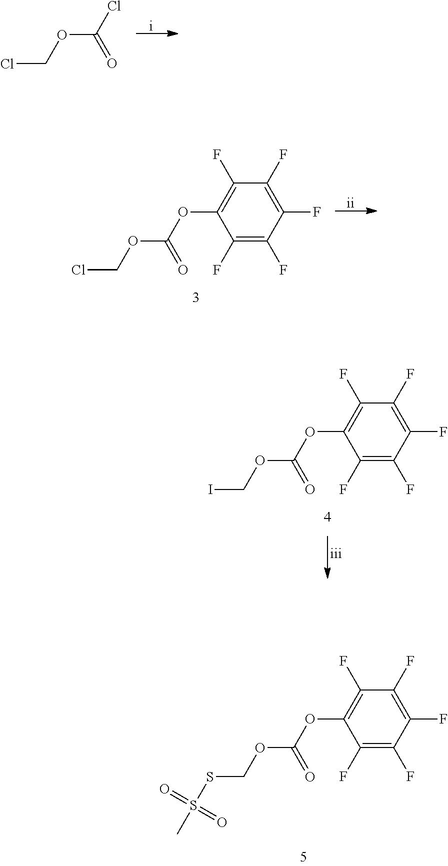

### Synthesis of Compound 3

At 0° C., a solution of 2,3,4,5,6-pentafluorophenol (5 g, 27.2 mmol, 1 eq.) and pyridine (base) (2.19 mL) in DCM (27.4 mL) was added in the course of 10 min to a solution of chloromethyl chloroformate [being of formula ClC(═O)OCH(R1)Cl wherein R1 is H](2.66 mL, 29.9 mmol, 1.1 eq.) in 54.3 mL of DCM. The reaction mixture was stirred at 0° C. under an inert atmosphere of nitrogen for 3 hours. The reaction mixture was washed with water, a 1 M solution of NaOH and then brine. The organic layer was dried over MgSO₄, filtered and evaporated to dryness yielding chloromethyl (2,3,4,5,6-pentafluorophenyl) carbonate 3 (7.42 g, 26.9 mmol, 99%) as an oil. ¹H-NMR (400 MHz; CDCl₃): δ 5.84 (s, 2H).

### Synthesis of Compound 4

A suspension of 3 (7.21 g, 26.1 mmol, 1 eq.), NaI (7.91 g, 53 mmol, 2 eq.) and NaHCO₃ (base) (437 mg, 5.20 mmol, 0.2 eq.) in 61 ml of acetone was stirred at 40° C. under an inert atmosphere of nitrogen for 24 hours. The precipitate was filtered off and washed with acetone. The filtrate was concentrated. The crude product was dissolved in EtOAc washed with water, a saturated solution of sodium thiosulfate and brine and dried over Na₂SO₄. The organic layer was concentrated yielding iodomethyl (2,3,4,5,6-pentafluorophenyl) carbonate 4 (9.13 g, 23.1 mmol, 89%) as a yellow oil. This compound can be stored in the freezer for months. ¹H-NMR (400 MHz; CDCl₃): δ 6.07 (s, 2H).

### Synthesis of Compound 5

A solution of sodium methanethiosulfonate (36.5 mg, 0.27 mmol, 1 eq.) and iodomethyl (2,3,4,5,6-pentafluorophenyl) carbonate (4) in DMF (1.83 mL) was stirred at room temperature for 45 min under an inert atmosphere of nitrogen. The reaction was finished according to the TLC (EtOAc/Hept 1/1). EtOAc was added to the reaction mixture. The reaction mixture was then washed with a saturated solution of sodium thiosulfate and with brine, dried and concentrated yielding compound methylsulfonylsulfanylmethyl (2,3,4,5,6-pentafluorophenyl) carbonate 5 (quantitative yield) as a yellow oil. The product was used without further purification.

### Second Protocol—Steps a and b

The below synthesis is a part of the synthesis of a compound according to Formula Ia wherein R1 is hydrogen and R28 is methyl. Step a according to claim 11, i.e. providing a reagent compound according to claim 9, was previously described. Step b according to claim 11 is shown below. This is a different way of preparing compounds 2 according to Formula IX.

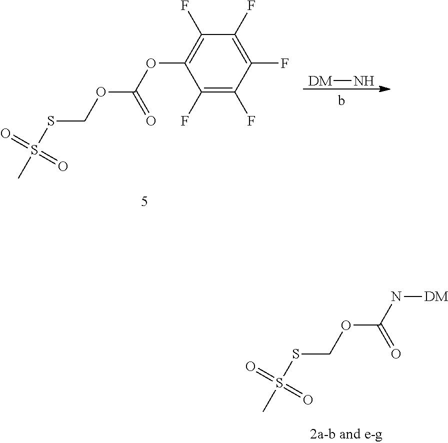

### Synthesis of Compounds 2

A freshly prepared solution of methylsulfonylsulfanylmethyl (2,3,4,5,6-pentafluorophenyl) carbonate 5 (2.22 mmol, 1.5 eq.), amine containing drug (1.48 mmol, 1 eq.) and triethylamine (206 μL) in an appropriate solvent (17 mL) was stirred at room temperature under an inert atmosphere of nitrogen until completion. For less reactive amine drugs, such as Cinacalet, HOBt (1 eq.) was added to speed up the reaction. A solvents selected from DCM, MeOH, THF or DMF is used. The reaction mixture was diluted DCM, washed with a saturated solution of NH₄Cl (twice). The aqueous layer was extracted then again with DCM. The organic layers were combined and dried over MgSO₄, filtered, and evaporated to dryness. The crude product was purified by column chromatography yielding compounds 2. Several drugs have been tested; Tables 2a in FIG. 5a show the compounds 2 that have been prepared. Table 2a-1: compounds 2a-c/Table 2a-2: compounds 2d-f/Table 2a-3: compounds 2g-i.

### First Protocol, Steps A and B

The below synthesis is a part of the synthesis of a compound according to Formula I.

In this case Z was O, R1 was hydrogen and R28 was methyl. The steps A and B according to claim 12.

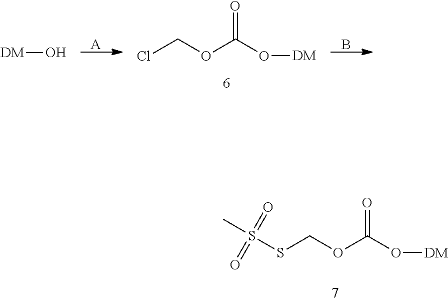

### Synthesis of Compounds 6

To a solution of a hydroxy containing drug molecule (0.71 mmol, 1 eq.) and DIPEA (282 μL) in DCM (4.6 mL) at room temperature was slowly added chloromethyl chloroformate (0.9 mmol, 1.3 eq.) under an inert atmosphere of nitrogen. The reaction mixture was stirred at room temperature under N: until completion. The reaction mixture was diluted in DCM, washed with water, a saturated solution of NaHCO₃ and brine and dried over MgSO₄. The crude material was purified by column chromatography yielding compound 6 according to Formula X. This product was used in the next step to yield a compound 7 (According to Formula XI). One specific drug (Abiraterone) was tested; however other drugs having an hydroxyl function may be used according to this synthesis.

### Synthesis of Compounds 7

A solution of sodium methanethiosulfonate (18 mg, 0.14 mmol, 1.2 eq.) and compound 6 (0.11 mmol, 1 eq.) in DMF (0.76 mL) was stirred at 70° C. After completion, EtOAc was added to the reaction mixture. The reaction mixture was then washed with brine, dried and concentrated. The crude product was purified by Flash chromatography yielding compound 7 (According to Formula XI). One specific drug (Abiraterone) was tested; however other drugs having an hydroxyl function may be used according to this synthesis. See Table 2b below for compound 7.

TABLE 2b
Intermediate thiosulfate-drug conjugates compound 7

| | | | | | | Retention time |
|---|---|---|---|---|---|---|
| | | | | | | (min) |
| Comp. | Drug | | | Protocol | | & |
| # | (DM[ZH]) | R1 | R2 | tested | Intermediate structure | m/z (LC-MS) |
| 7 | Abiraterone | H | CH₃ | First | 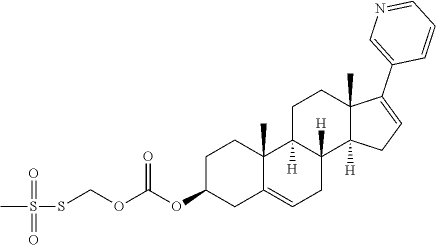 | Not determined |

Coupling of Intermediate Thiosulfate-Drug Conjugates (Also Called Alkyl- or Aryl-Sulfonylsulfanylmethyl-Drug Conjugate) with Commercially Available Organic Moiety G[C]—SH
The below synthesis is a part of the synthesis of a compound according to Formula I or Ia. The step C is according to claim 12 and the step c according to claim 11.

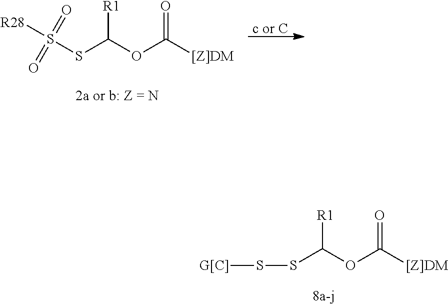

### First Protocol—Step C

### Synthesis of Compound 8

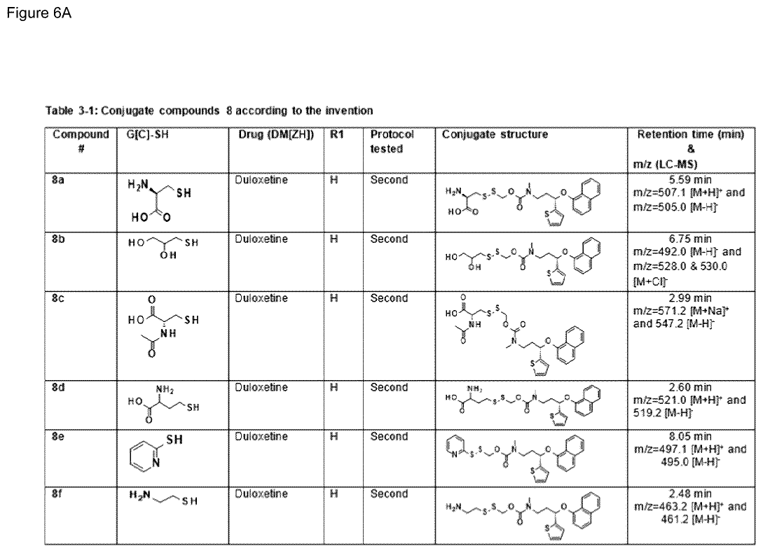

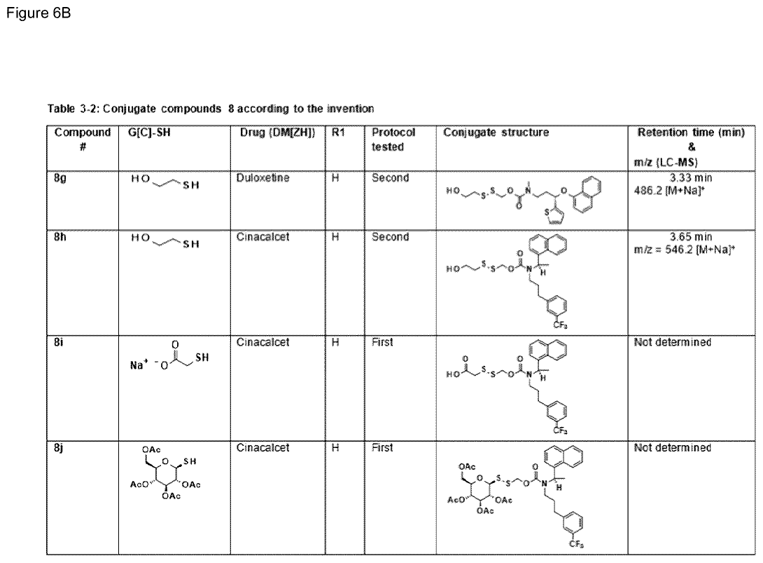

To a solution of a compound 2b (0.38 mmol, 1 eq.) and an organic moiety G[C]—SH (0.38 mmol, 1 eq.) in 4 ml of a solvent, such as MeOH, THF or DMF, a solution of NaHCO₃ (base) (0.38 mmol, 1 eq.) in water (1.8 mL) was slowly added. The reaction mixture was stirred under an inert atmosphere of nitrogen. After completion, the reaction mixture was then diluted with EtOAc and washed with water and brine. The organic layer was dried, concentrated, and purified by flash chromatography to give compounds 8i-j. (according to Formula I). Tables 3 in FIGS. 6A-B show the compounds 8i-j that have been prepared. Table 3-1: compounds 8a-f/Table 3-2: compounds 8g-j. The compounds of Tables 3-1 and 3-2 comprise a group G that is according to Formula IV.

### Second Protocol—Step c

### Synthesis of Compound 8

A solution of a compound 2a or b (1.12 mmol) and an organic moiety G[C]—SH (1.46 mmol; 1.3 eq.) in a solvent, such as THF or DMF, was stirred at room temperature under an inert atmosphere of nitrogen till completion. The reaction mixture was concentrated and purified by column chromatography yielding compounds 8a-h (according to Formula Ia). Tables 3 show the compounds 8a-h that have been prepared.

### Optional Step of Deprotection of Hydroxyl Groups in G[C] Moiety

### First Protocol—Step D or Second Protocol—Step d

The below synthesis is one specific example (going from compound 8j to compound 9) of an optional step in the synthesis of a compound according to Formula I or Ia. The step D can be present as an optional step in claim 12, after step C and the step d can be present as an optional step in claim 11, after step d.

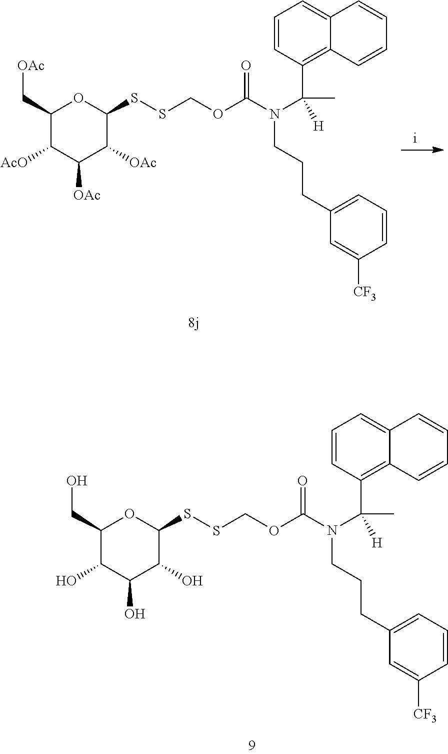 [SMILES: CC(=O)OC[C@H]1O[C@@H](SSCOC(=O)N(CCCc2cccc(C(F)(F)F)c2)[C@H](C)c2cccc3ccccc23)[C@H](OC(C)=O)[C@@H](OC(C)=O)[C@@H]1OC(C)=O ; C[C@H](c1cccc2ccccc12)N(CCCc1cccc(C(F)(F)F)c1)C(=O)OCSS[C@@H]1O[C@H](CO)[C@@H](O)[C@H](O)[C@H]1O]

### Synthesis of Compound 9

To a solution of compound 8j (408 mg, 0.5 mmol, 1 eq.) in MeOH (10 mL) was added NaOMe (27 mg, 0.5 mmol, 1 eq.). The reaction was stirred at room temperature under an inert atmosphere of nitrogen until completion. A saturated solution of NaHCO₃ was added to the reaction mixture. The product was extracted with EtOAc. The organic layers were combined, dried and concentrated yielding 9 (306 mg, 0.48 mmol, 95%). LC-MS (ESI): r.t.=3.16 min, m/z calcd. for C₃₀H₃₄F₃NO₇S₂=641.2; found m/z=664.4 [M+Na]⁺, m/z=686.2 [M−H+HCOOH]⁻.

Preparation of Organic Moiety G[C]—SH; which Optionally have Protection Groups on the Hydroxyl Groups

The below synthesis shows the formation of a specific galactose O-linked thiol compound 12.

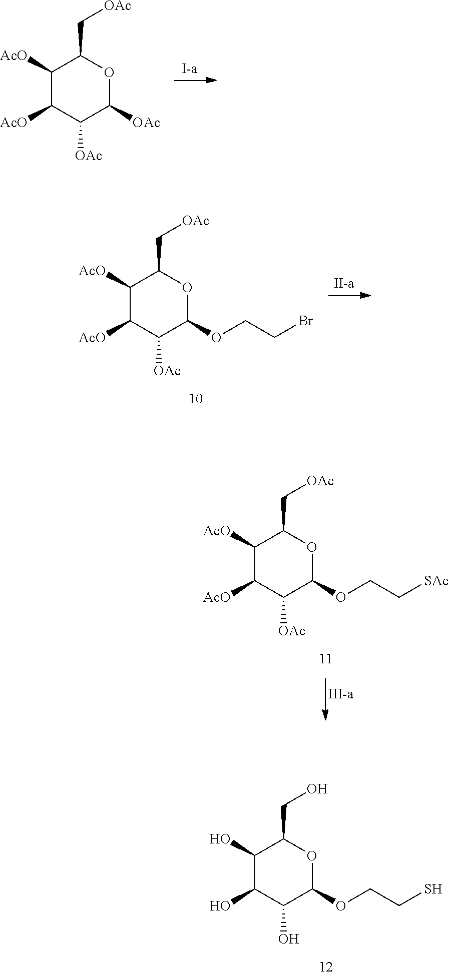

### Synthesis of Compound 10

Dry penta-O-acetyl-β-D-galactopyranoside (5.0 g, 13 mmol, 1.0 eq.) was dissolved in anhydrous DCM (30 mL) and cooled to 0° C. under an inert atmosphere of nitrogen. 2-bromoethanol (1.8 mL, 26 mmol, 2.0 eq.) was added, followed by the dropwise addition of boron trifluoride diethyletherate (4.7 mL, 38 mmol, 3.0 eq.). The reaction was warmed up to room temperature. After 1 h at ambient temperature, the reaction mixture was diluted with EtOAc, then washed with a saturated solution of NaHCO₃ saturated and brine. The organic layer was dried with MgSO₄ and concentrated under vacuum. The crude mixture was purified by flash chromatography (silica, 10->70% EtOAc in heptane) to afford the desired compound 10 as a transparent oil that crystallised over time (4.25 g, 9.34 mmol, 73%).

### Synthesis of Compound 11

To a solution of the bromide 10 (4.25 g, 9.34 mmol, 1.0 eq.) in DMF (12 mL) under an inert atmosphere of nitrogen was added potassium thioacetate (1.6 g, 14 mmol. 1.5 eq.). The reaction mixture was stirred under N₂ at 80° C. until full conversion. The crude mixture was dissolved in EtOAc (100 mL), washed with brine, a 2 M solution of NaOH and brine. The organic layer was dried over MgSO₄, filtered and evaporated to dryness. The crude mixture was purified by flash chromatography (silica, 5->70% EtOAc in heptane) to afford the desired product 11 as a pale orange oil (3.73 g, 8.28 mmol, 88%).

### Synthesis of Compound 12

To a solution of 11 (700 mg, 1.54 mmol, 1 eq.) in MeOH (10 mL), NaOMe (84 mg, 1.54 mmol, 1 eq.) was added. The reaction was stirred under an inert atmosphere of nitrogen for 6 h. The reaction mixture was neutralized with Dowex® (50W×8 100-200 mesh), filtered and concentrated in vacuo yielding 12 as a clear oil in a quantitative yield. Compound 12 can be used as a G[C]—SH compound for preparing compounds according to the present invention.

Using the same method as discussed above for compound 12, the formation of a specific glucose O-linked thiol compound 13 can be carried out.

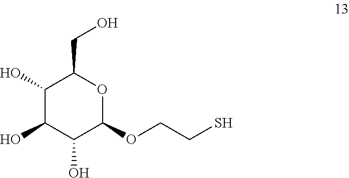 [SMILES: OC[C@H]1O[C@@H](OCCS)[C@H](O)[C@@H](O)[C@@H]1O]

### Synthesis of Compound 13

Compound 13 was obtained in a similar manner to that described for compound 12 starting with penta-O-acetyl-β-D-glucopyranoside.

The below synthesis shows the formation of a specific glucose O-linked thiol compound 17.

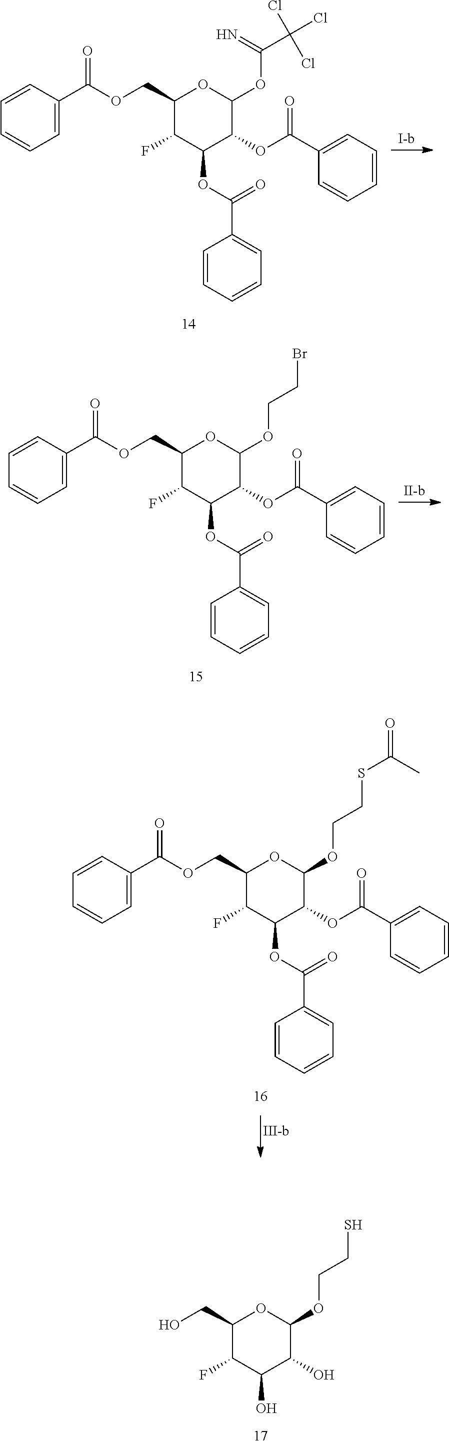 [SMILES: N=C(OC1O[C@H](COC(=O)c2ccccc2)[C@@H](F)[C@H](OC(=O)c2ccccc2)[C@H]1OC(=O)c1ccccc1)C(Cl)(Cl)Cl ; O=C(OC[C@H]1OC(OCCBr)[C@H](OC(=O)c2ccccc2)[C@@H](OC(=O)c2ccccc2)[C@@H]1F)c1ccccc1 ; CC(=O)SCCO[C@@H]1O[C@H](COC(=O)c2ccccc2)[C@@H](F)[C@H](OC(=O)c2ccccc2)[C@H]1OC(=O)c1ccccc1 ; OC[C@H]1O[C@@H](OCCS)[C@H](O)[C@@H](O)[C@@H]1F]

### Synthesis of Compound 15

To a solution of 2,3,6-tri-O-benzoyl-4-fluoro-4-deoxy-D-glucopyranosyl trichloro acetimidate 14 (disclosed in WO2010/77623) (4.1 g, 6.4, 1 eq.) in DCM (20.0 mL) containing 4 Å MS was added 2-bromoethanol (2.4 g, 19.3 mmol, 3 eq.) and the solution was cooled to 0° C. To this solution was added BF₃·O(C₂H₅)₂ (0.95 mL, 7.7 mmol, 1.2 eq.) and the resulting solution stirred at 0° C. for 2 h. After this time Et₃N was added and the solution filtered, concentrated and purified by flash chromatography to give 15 (2.32 g, 3.9 mmol, 60%).

### Synthesis of Compound 16

Compound 16 was obtained in a similar manner to that described for compound 11 starting with 15. Yield=quantitative

### Synthesis of Compound 17

Compound 17 was obtained in a similar manner to that described for compound 12 starting with 16. Yield=quantitative.

The below synthesis shows the formation of a specific O-linked thiol compound 23.

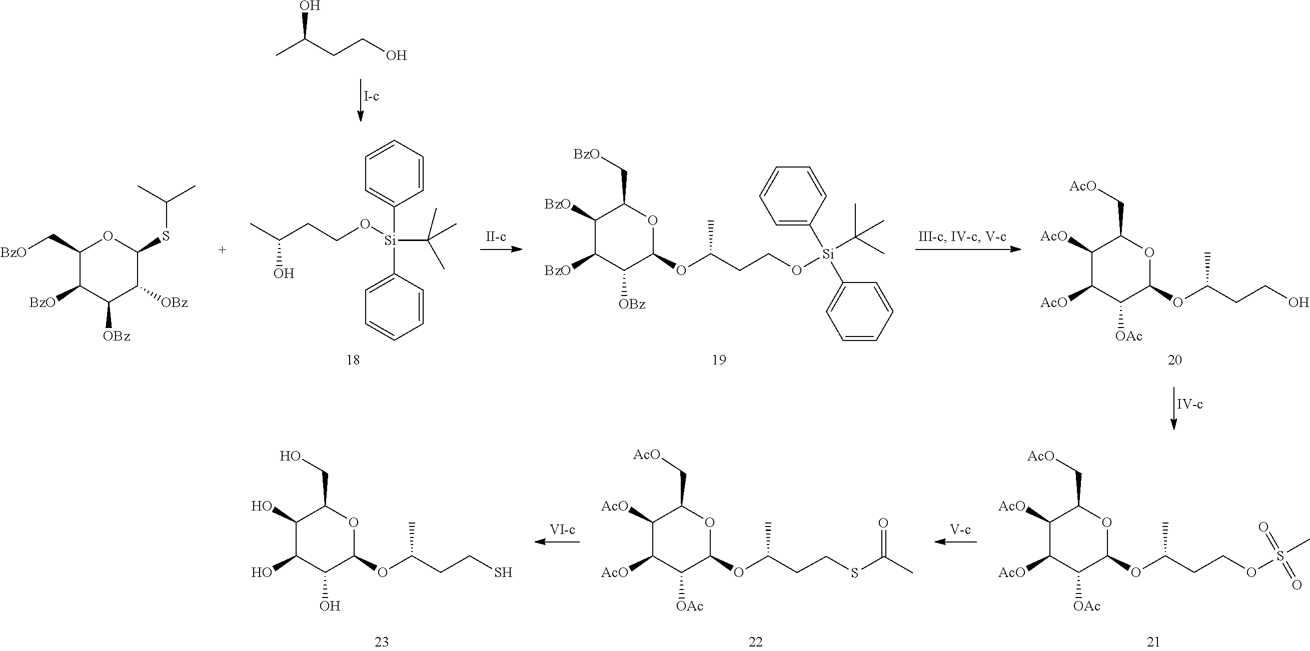

### Synthesis of Compound 18

To a solution of (3R)-butane-1,3-diol (2.25 g, 25 mmol, 1 eq.) in pyridine (14 mL) stirred at 0° C. was slowly added TBDMS-Cl (7.1 mL, 27.5 mmol, 1.1 eq) followed by DMAP (31 mg, 0.25 mmol, 0.1 eq.). The reaction mixture was stirred for 18 h at room temperature under an inert atmosphere of nitrogen. The reaction mixture was diluted with water and extracted several times with DCM. The organic layers were combined, dried over MgSO₄ and concentrated yielding compound 18 (6.8 g, 20.6 mmol, 82%).

### Synthesis of Compound 19

To a solution of isopropyl 2,3,4,6-tetra-O-benzoyl-R-D-1-thiogalactopyranose (17.5 g, 27 mmol, 1.3 eq.), compound 18 (6.8 g, 21 mmol, 1 eq.) and molecular sieves (4 Å) in DCM (104 mL) was added NIS (7 g, 31 mmol, 1.5 eq.) and triflic acid (183 mL, 2.1 mmol, 0.1 eq.). The solution was stirred at room temperature under an inert atmosphere of nitrogen for a few minutes. The reaction mixture was diluted with DCM and washed with a solution of sodium thiosulfate and a saturated solution of NaHCO₃. The organic layer was dried over MgSO₄, filtered, evaporated to dryness and purified by column chromatography yielding 19 (13.9 g, 15.3 mmol, 74%).

### Synthesis of Compound 20

Step III-c): To a solution of compound 19 (13.8 g, 15.2 mmol, 1 eq.) in MeOH (40 mL) and dioxane (40 mL) was added NaOMe (1.6 g, 30 mmol, 2 eq.) and the resulting solution was stirred at room temperature until completion. The reaction mixture was neutralized with Dowex H⁺, filtered and concentrated. The residue was coevaporated with pyridine and used as such in the next step.

Step IV-c): To the material obtained in the previous step (15.2 mmol, 1 eq.) in pyridine (80 mL) was added acetic anhydride (8.6 mL, 91.2 mmol, 6 eq.). The reaction mixture was stirred overnight at room temperature under an inert atmosphere of nitrogen. The solution was concentrated and coevaporated with toluene. The residue was dissolved in EtOAc and washed with a 1M solution of HCl, water, a saturated solution NaHCO₃ and brine. The organic layers were dried over MgSO₄, filtered and purified by flash chromatography (silica, 0→50% EtOAc in heptane) yielding the desired product (7.2 g, 11 mmol, 72%).

Step V-c): To the material obtained in the previous step (7.2 g, 11 mmol, 1 eq.) in THF (75 mL) was added acetic acid (627 μL, 11 mmol, 1 eq.) and a 1 M solution of TBAF in THF (11 mL, 11 mmol, 1 eq.). The reaction mixture was stirred at room temperature for 24 h. The solution was then concentrated and purified by flash chromatography (silica, 20→100% EtOAc in heptane) to give compound 20 (3.7 g, 8.9 mmol, 81%).

### Synthesis of Compound 21

To a solution of compound 20 (3.4 g, 8.1 mmol, 1 eq.) in pyridine (65 mL) was added MsCl (1.3 mL, 16.3 mmol, 2 eq.) at 4° C. under an inert atmosphere of nitrogen. The reaction mixture was then stirred at room temperature under N₂ for 1 h. The mixture was concentrated, dissolved in EtOAc and filtered. The filtrate was washed with a 0.5 M solution of HCl, water, and a saturated solution of NaHCO₃. The organic layer was dried over MgSO₄, filtered and evaporated to dryness yielding compound 21 (3.6 g, 7.2 mmol, 89%). LC-MS (ESI): r.t.=2.64 min, m/z calcd. for C₁₉H₃₀O₁₃S=498.1; found m/z=521.2 [M+Na]⁺.

### Synthesis of Compound 22

To a solution of the compound 21 (3.6 g, 7.2 mmol, 1 eq.) in DMF (15 mL), potassium thioacetate (2.6 g, 22.4 mmol, 3.1 eq.) was added. The reaction mixture was stirred at 50° C. for 1 h under an inert atmosphere of nitrogen. The reaction mixture was the diluted with EtOAc and washed with water, and brine. The organic layer was dried over MgSO₄, filtered, concentrated in vacuo and purified by column chromatography (silica, 0->60% EtOAc in heptane) yielding compound 22 (1.9 g, 4.0 mmol, 56%) as a red oil.

### Synthesis of Compound 23

To a solution of 22 (574 mg, 1.2 mmol, 1 eq.) in MeOH (10 mL) was added NaOMe (130 mg, 2.4 mmol, 2 eq.) and the resulting solution stirred at room temperature under an inert atmosphere of nitrogen until completion. The reaction mixture was neutralized with Dowex H⁺, filtered and concentrated to give 23 (320 mg, 1.2 mmol, 99%). LC-MS (ESI): r.t.=0.64 min, m/z calcd. for C₁₀H₂₀O₆S=268.1; found m/z=291.0 [M+Na]⁺, m/z=267.0 [M−H]− and m/z=313.0 [M−H+HCOOH]⁻.

The below synthesis shows the formation of a specific O-linked thiol compound 28.

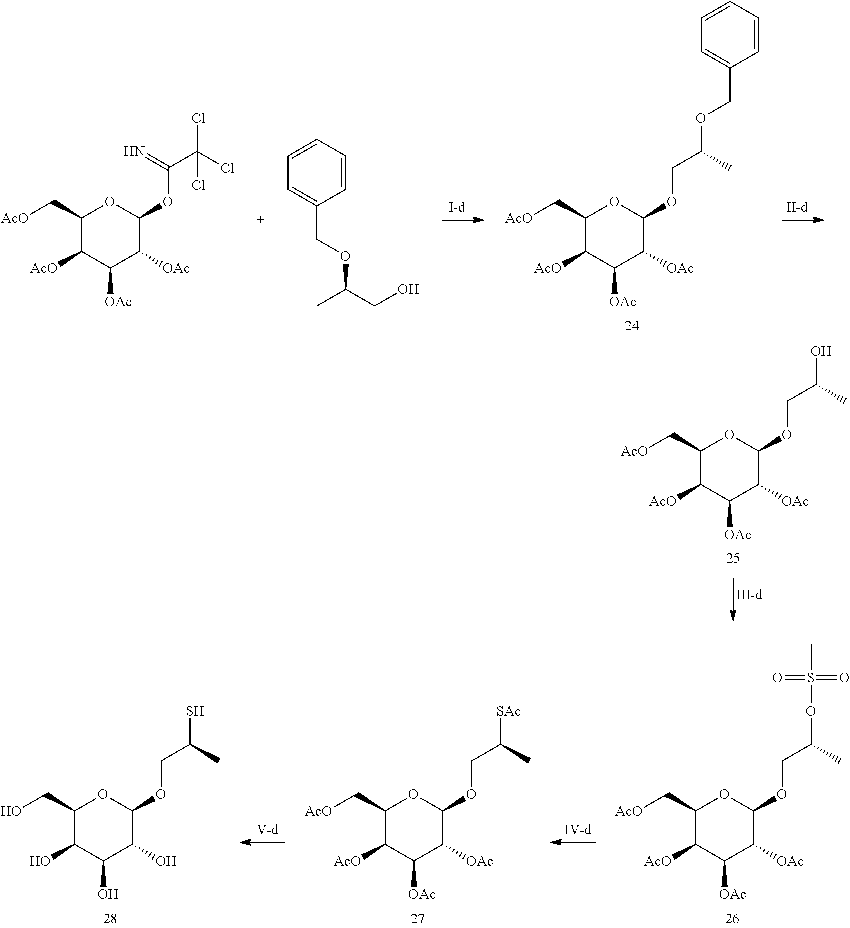

### Synthesis of Compound 24

To a solution of dry 2,3,4,6-tetra-O-acetyl-D-galactopyranosyl trichloroacetimidate (4.4 g, 8.9 mmol, 1 eq.) and (R)-2-(benzyloxy)-propan-1-ol (1.5 g, 8.9 mmol, 1 eq.) in DCM (20 mL) was added BF₃·O(C₂H₅)₂ (1.6 mL, 13.4 mmol, 1.5 eq.) at −10° C. under an inert atmosphere of nitrogen. The reaction mixture was then allowed to slowly warm up to room temperature. After 2 h, TEA (2.10 mL, 15.1 mmol, 1.7 eq.) was added and the solution was filtered. DCM (50 mL) was added to the filtrate. The mixture was washed with a saturated solution of NaHCO₃ and brine, dried over MgSO₄ and concentrated. The crude product was purified by flash chromatography (silica, 0->50% EtOAc in heptane) yielding 21 (2.5 g, 5.1 mmol, 57%) as a slightly yellow oil. LC-MS (ESI): r.t.=2.99 min, m/z calcd. for C₂₄H₃₂O₁₁=496.2; found m/z=519.2 [M+Na]⁺.

### Synthesis of Compound 25

Compound 24 (2.5 g, 5.1 mmol, 1 eq.) was dissolved in MeOH (50 ml) and 54 mg Pd/C was added. The reaction was stirred at room temperature under H₂. Upon completion, the reaction mixture was filtered through celite and concentrate in vacuo yielding 25 (2.0 g, 5 mmol, 98%) as a slightly yellow resin. The compound 25 was used in the next step without further purification.

### Synthesis of Compound 26

The alcohol 25 (1.2 g, 3 mmol, 1 eq.) was dissolved in dry DCM (12 mL) under an inert atmosphere of nitrogen. TEA (418 μL, 3 mmol, 1 eq.) was added to the reaction mixture followed by mesyl chloride (232 μL, 3 mmol, 1 eq.). The reaction mixture was stirred at room temperature till completion. The mixture was diluted with DCM, washed with water and brine, dry over MgSO₄ and concentrated to give compound 26 (1.45 g, 3 mmol, quantitative). Compound 26 was used without further purification in the next step.

### Synthesis of Compound 27

To a solution of compound 26 (1.45 g, 3 mmol, 1 eq.) in DMF (20 mL) under an inert atmosphere of nitrogen was added potassium thioacetate (2.4 g, 21 mmol, 7 eq.). The reaction mixture was stirred under N₂ at 80° C. for 1 hour. The reaction mixture was then concentrated and dissolved in EtOAc. The solution was washed with brine, dried over MgSO₄ and concentrated to dryness. The crude material was purified by flash chromatography (silica, 0→50% EtOAc in Heptane) to give 27 (950 mg, 2.05 mmol, 68%) as a dark red/brown resin.

### Synthesis of Compound 28

Deacetylation of compound 28 was performed in a similar manner to that described for compound 23.

The below synthesis shows the formation of a specific hydroxy intermediate 30a, 30b, 30c or 30d.

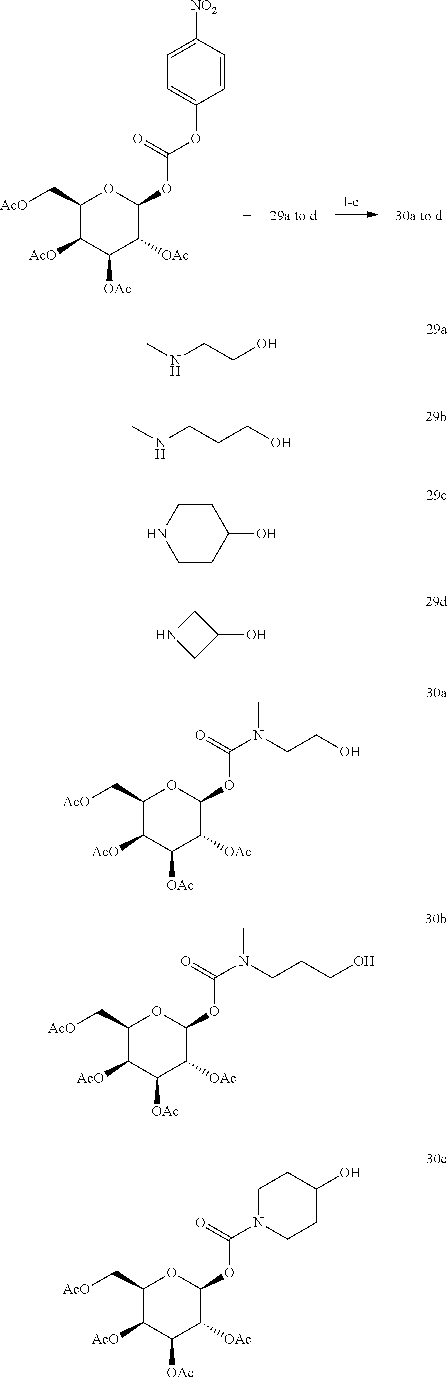

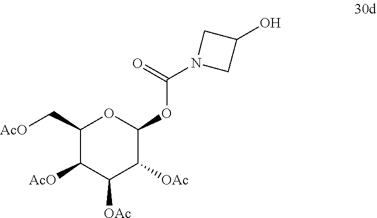 [SMILES: CC(=O)OC[C@H]1O[C@@H](OC(=O)N2CC(O)C2)[C@H](OC(C)=O)[C@@H](OC(C)=O)[C@H]1OC(C)=O]

### Synthesis of Compound 30

To a solution of p-nitrophenyl 2,3,4,6-tetra-O-acetyl-β-D-galactopyranosyl carbonate (3.9 mmol, 1 eq.) (see Bioorg. Med. Chem. Lett., 2016, 26, 3774) and 29a or 29b or 29c or 29d (3.9 mmol, 1 eq.) in DCM (55 mL) was added TEA (7.8 mmol, 2 eq.). The solution was stirred at room temperature under an inert atmosphere of nitrogen until completion. The reaction mixture was diluted with DCM and washed with a 1 M solution of HCl and brine. The organic layer was dried over MgSO₄, filtered and evaporated to dryness. The crude compound was purified by column chromatography yielding the compounds 30a or 30b or 30c or 30d. Compound 30a was obtained in a yield of 94% (2.44 min; m/z=472.2 [M+Na]⁺). Compound 30b was obtained in a quantitative yield (2.46 min; m/z=486.2 [M+Na]⁺). Compound 30c was obtained in a yield of 86% (2.47 min; m/z=514.2 [M+K]⁺). Compound 30d was obtained in a yield of 81% (2.36 min; m/z=470.2 [M+Na]⁺).

The below synthesis shows the formation of specific thioalkyl-linked carbamates 33a, 33b, 33c and 33d.

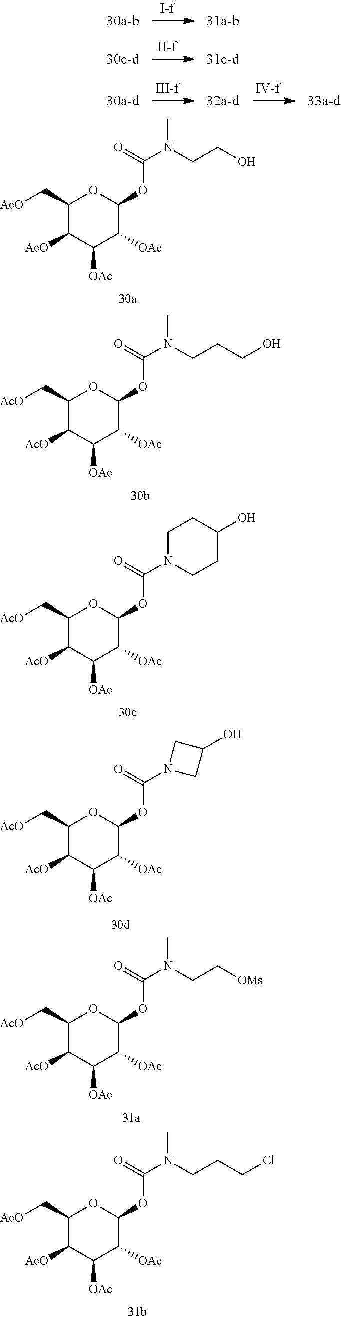

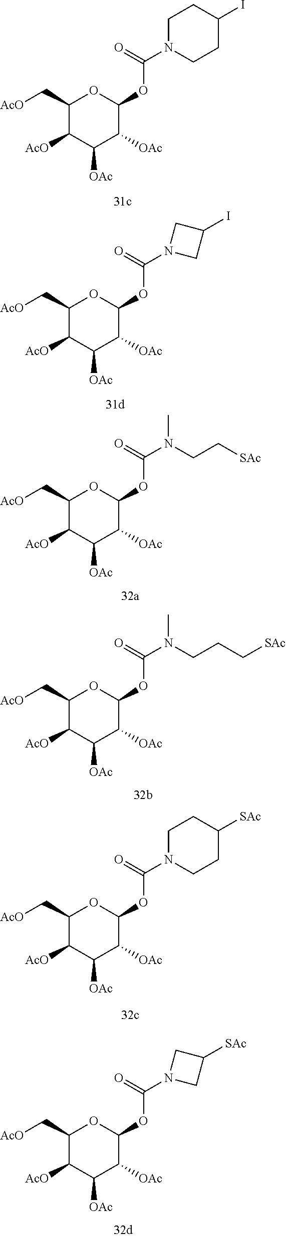

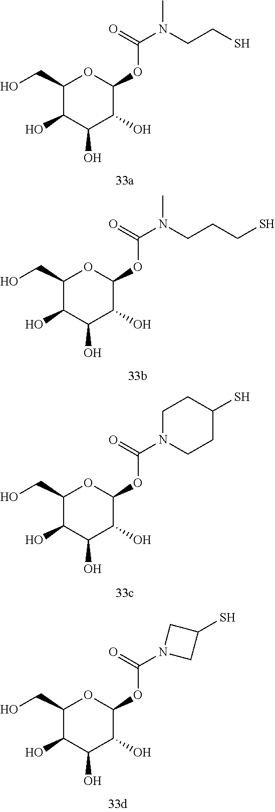 [SMILES: CN(CCS)C(=O)O[C@@H]1O[C@H](CO)[C@H](O)[C@H](O)[C@H]1O ; CN(CCCS)C(=O)O[C@@H]1O[C@H](CO)[C@H](O)[C@H](O)[C@H]1O ; O=C(O[C@@H]1O[C@H](CO)[C@H](O)[C@H](O)[C@H]1O)N1CCC(S)CC1 ; O=C(O[C@@H]1O[C@H](CO)[C@H](O)[C@H](O)[C@H]1O)N1CC(S)C1]

### Synthesis of Compounds 31a or 31b

To a solution of 30a or b (3.67 mmol, 1 eq.) in pyridine (16.6 mL) was added methanesulfonyl chloride (7.34 mmol, 2 eq.) at 4° C. The reaction mixture was then stirred at room temperature under an inert atmosphere of nitrogen until completion. The reaction mixture was concentrated. The residue was dissolved in EtOAc. The suspension was filtered. The filtrate was washed with a 0.5 M solution of HCl, water, and a saturated solution of NaHCO₃. The organic layer was dried over MgSO₄ and evaporated to dryness yielding 31a or b. Compound 31a was obtained as an oil from 30a. Yield=98%. LCMS (ESI): rt=2.61 min, m/z calcd for C₁₉H₂₉NO₁₄S=527.13, found m/z=550.2 [M+Na]⁺ Compound 31b was obtained as an oil from 30b. Yield=86%. LCMS (ESI): rt=2.815 min, m/z calcd for C₁₉H₂₈ClNO₁₁=481.1, found m/z=504.2 & 506.2 [M+Na]⁺.

### Synthesis of Compounds 31c or d

To a solution of 30c or 30d (8.5 mmol, 1 eq.) in toluene (85 mL) were added successively imidazole (25.6 mmol, 3 eq.), PPh₃ (17.1 mmol, 2 eq.) and 12 (12.8 mmol, 1.5 eq.). The reaction mixture was stirred at 110° C. until completion. The reaction mixture was quenched by adding a saturated solution of NaHCO₃. The aqueous layer was extracted with EtOAc. 1₂ was added to the combined organic phases until a persistent brown color was observed. The organic phase was washed with a saturated aqueous solution of Na₂S₂O₃, dried over Na₂SO₄, filtered and concentrated under reduce pressure. The product was purified by flash chromatography yielding 31c or 31d. Compound 31c was obtained from 30c. Yield=74%. LCMS (ESI): rt=3.02 min, m/z calcd for C₂₀H₂₈INO₁₁=585.0, found m/z=608.2 [M+Na]⁺. Compound 31d was obtained from 30d. Yield=quantitative. LCMS (ESI): rt=2.80 min, m/z calcd for C₁₈H₂₄INO₁₁=557.0, found m/z=580.0 [M+Na]⁺.

### Synthesis of Compounds 32a, 32b, 32c, or 32d

To a solution of 31 (a to d) (2.9 mmol, 1 eq.) in DMF (6.5 mL) under N₂ was added potassium thioacetate (8.9 mmol, 3 eq.). The reaction mixture was stirred under N₂ at 50° C. until completion. The reaction mixture was diluted with EtOAc and washed with brine (3×). The organic layer was dried over MgSO₄, filtered and evaporated to dryness. The crude compound was purified by column chromatography. Compound 32a was obtained as a brown oil from 31a. Yield=77%. LCMS (ESI), rt=2.78 min, m/z calcd for C₂₀H₂₉NO₁₂S=507.1, found m/z=530.2 [M+Na]⁺ Compound 32b was obtained as a brown oil from 31b. Yield=95%. LCMS (ESI), rt=2.82 min, m/z calcd for C21H₃₁NO₁₂S=521.2, found m/z=544.2 [M+Na]⁺. Compound 32c was obtained as a clear oil from 31c. Yield=81%. Compound 32d was obtained from 31d. Yield=92%. LCMS (ESI): rt=2.65 min, m/z calcd for C₂₀H₂₇NO₁₂S=505.1, found m/z=528.2 [M+Na]⁺.

### Synthesis of Compounds 33a, 33b, 33c, or 33d

To a solution of 32 (a to d) (2.2 mmol, 1 eq.) in MeOH (13 mL) was added NaOMe (2.2 mmol, 1.5 eq.) under an inert atmosphere of nitrogen. The reaction was stirred at room temperature under an inert atmosphere of nitrogen until completion. The reaction mixture was neutralized with Dowex (H′) and filtered. The filtrate was evaporated to dryness yielding 33a to d. Compound 33a was obtained as a brown oil from 32a. Yield=quantitative. LCMS (ESI), rt=0.34 min, m/z calcd for C₁₀H₁₉NO₇S=297.1, found m/z=320.0 [M+Na]⁺. Compound 33b was obtained as a brown oil from 32b. Yield=quantitative. LCMS (ESI), rt=0.36 min, m/z calcd for C₁₁H₂₁NO₇S=311.1, found m/z=334.0 [M+Na]⁺ and m/z=310.0 [M−H]⁻ Compound 33c was obtained from 32c. Yield=98%. Compound 33d was obtained as slightly yellow oil from 32d. Yield=quantitative. LCMS (ESI), rt=0.34 min, m/z calcd for C₁₀H₁₇NO₇S=295.1, found m/z=318.0 [M+Na]⁺ and m/z=294.0 [M−H]⁻

The below synthesis shows the formation of specific thiol compounds 39a-c, and building blocks 40a-b and 41. First compounds 34, 35a, 35b, 35c, 36a, or 36b are prepared.

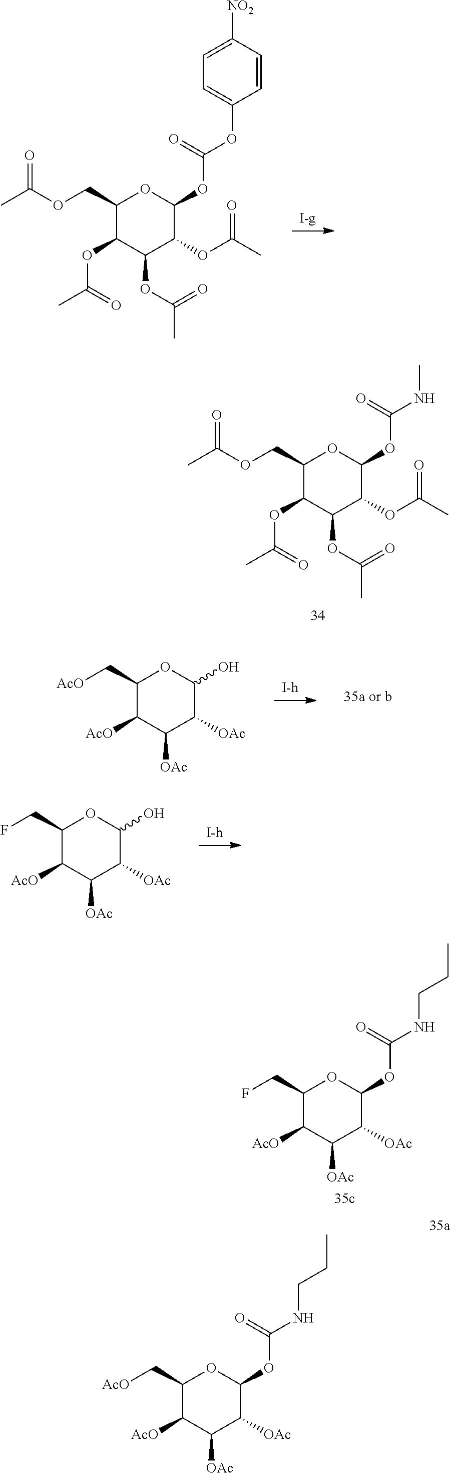 [SMILES: CC(=O)OC[C@H]1O[C@@H](OC(=O)Oc2ccc([N+](=O)[O-])cc2)[C@H](OC(C)=O)[C@@H](OC(C)=O)[C@H]1OC(C)=O ; CNC(=O)O[C@@H]1O[C@H](COC(C)=O)[C@H](OC(C)=O)[C@H](OC(C)=O)[C@H]1OC(C)=O ; CC(=O)OC[C@H]1OC(O)[C@H](OC(C)=O)[C@@H](OC(C)=O)[C@H]1OC(C)=O ; CC(=O)O[C@@H]1[C@H](OC(C)=O)[C@@H](OC(C)=O)C(O)O[C@@H]1CF ; CCCNC(=O)O[C@@H]1O[C@H](CF)[C@H](OC(C)=O)[C@H](OC(C)=O)[C@H]1OC(C)=O ; CCCNC(=O)O[C@@H]1O[C@H](COC(C)=O)[C@H](OC(C)=O)[C@H](OC(C)=O)[C@H]1OC(C)=O]

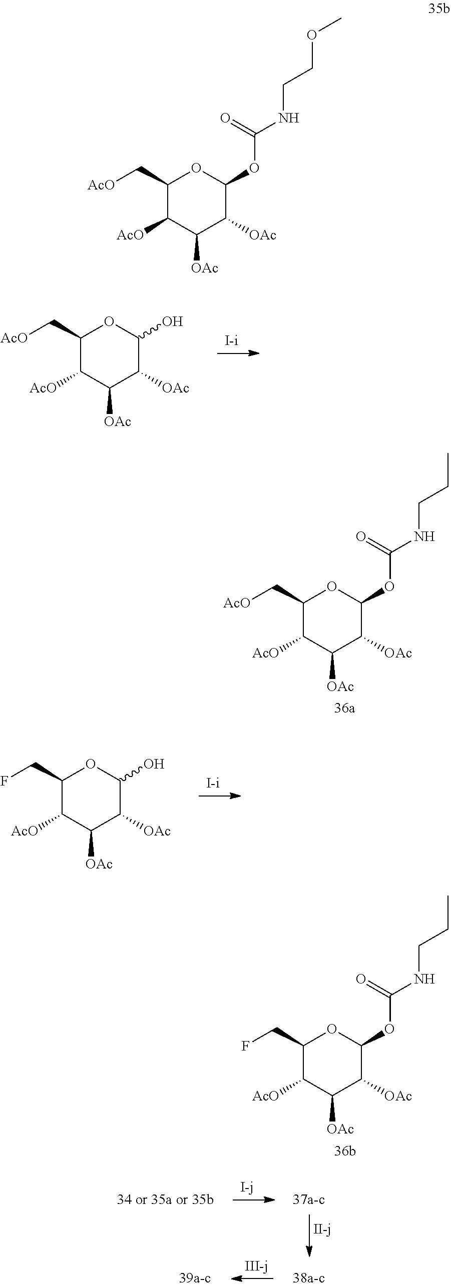 [SMILES: COCCNC(=O)O[C@@H]1O[C@H](COC(C)=O)[C@H](OC(C)=O)[C@H](OC(C)=O)[C@H]1OC(C)=O ; CC(=O)OC[C@H]1OC(O)[C@H](OC(C)=O)[C@@H](OC(C)=O)[C@@H]1OC(C)=O ; CCCNC(=O)O[C@@H]1O[C@H](COC(C)=O)[C@@H](OC(C)=O)[C@H](OC(C)=O)[C@H]1OC(C)=O ; CC(=O)O[C@H]1[C@H](OC(C)=O)[C@@H](OC(C)=O)C(O)O[C@@H]1CF ; CCCNC(=O)O[C@@H]1O[C@H](CF)[C@@H](OC(C)=O)[C@H](OC(C)=O)[C@H]1OC(C)=O]

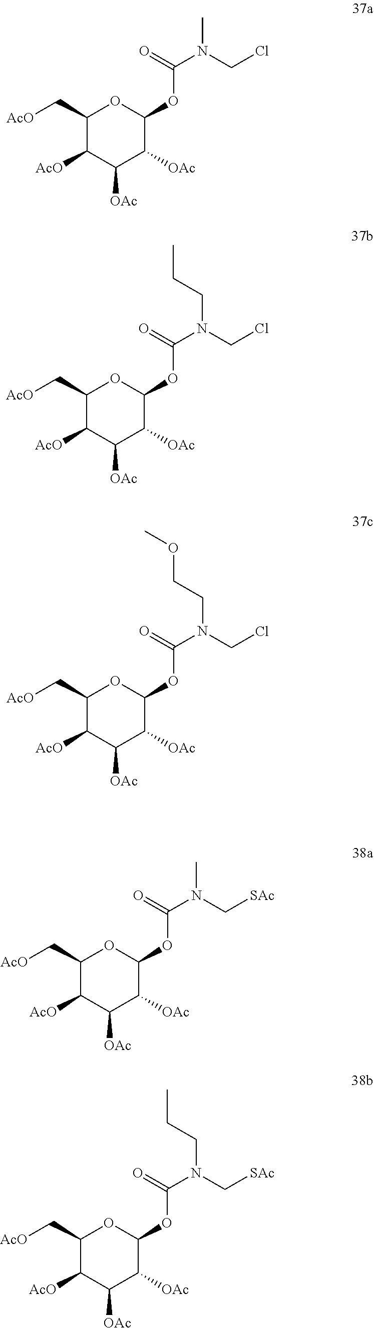

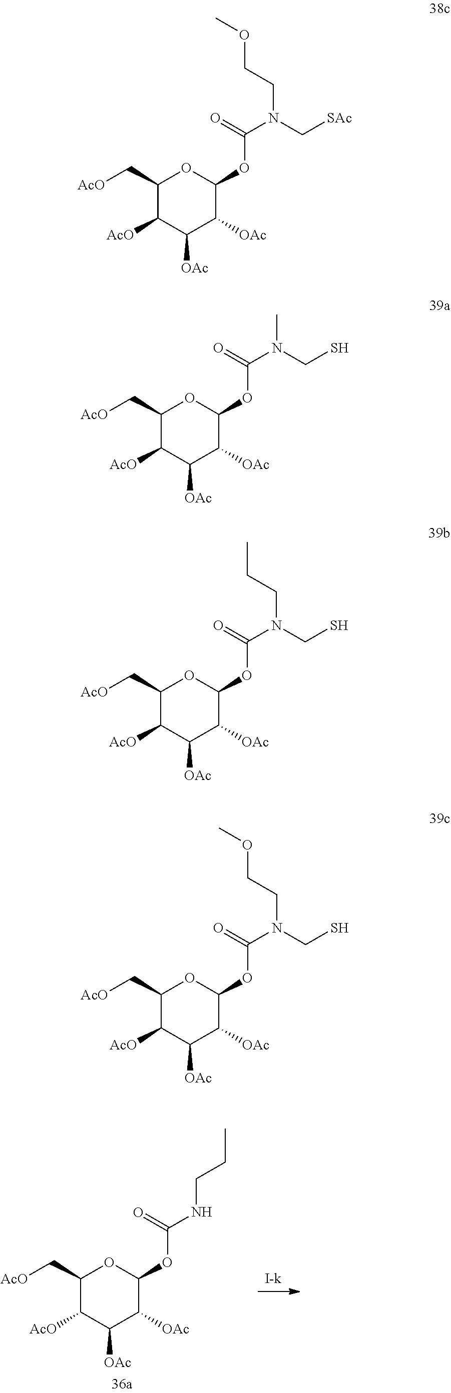

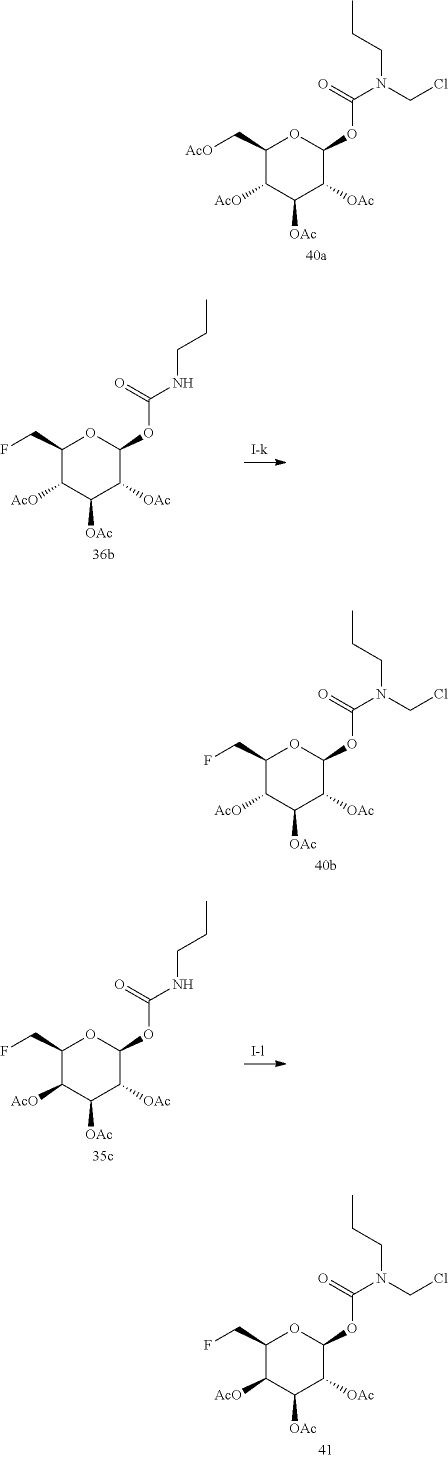 [SMILES: CCCN(CCl)C(=O)O[C@@H]1O[C@H](COC(C)=O)[C@@H](OC(C)=O)[C@H](OC(C)=O)[C@H]1OC(C)=O ; CCCNC(=O)O[C@@H]1O[C@H](CF)[C@@H](OC(C)=O)[C@H](OC(C)=O)[C@H]1OC(C)=O ; CCCN(CCl)C(=O)O[C@@H]1O[C@H](CF)[C@@H](OC(C)=O)[C@H](OC(C)=O)[C@H]1OC(C)=O ; CCCNC(=O)O[C@@H]1O[C@H](CF)[C@H](OC(C)=O)[C@H](OC(C)=O)[C@H]1OC(C)=O ; CCCN(CCl)C(=O)O[C@@H]1O[C@H](CF)[C@H](OC(C)=O)[C@H](OC(C)=O)[C@H]1OC(C)=O]

### Synthesis of Compound 34

To a solution of p-nitrophenyl 2,3,4,6-tetra-O-acetyl-β-D-galactopyranosyl carbonate (3 g, 5.84 mmol, 1 eq.) in DCM (54 mL) was added a 2M solution of methylamine in THF (20.5 mL, 40.9 mmol, 7 eq.). The reaction mixture turned yellow. The solution was stirred at room temperature under an inert atmosphere of nitrogen for 20 min. The reaction mixture was diluted with DCM, washed with a 1 M solution of HCl, a saturated solution of NaHCO₃, and brine. The organic layer was dried over MgSO4, filtered, and evaporated to dryness. The crude product was dissolved in DCM and purified by column chromatography (silica, 30% →70% EtOAc in heptane) yielding 34 (2.23 g, 5.5 mmol, 94%) as a white solid. LC-MS (ESI): r.t.=2.39 min, m/z calcd. for C16H₂₃NO₁₁=405.1; found m/z=428.2 [M+Na]⁺.

### Synthesis of Compounds 35a-c

The β-linked carbamate intermediates 35a, b and c were prepared from known 2,3,4,6-tetra-O-acetyl-D-galactopyranose or 2,3,4-tri-O-acetyl-6-deoxy-6-fluoro-D-galacto pyranose by reaction with appropriate isocyanates (2 eq) in toluene in the presence of triethylamine (1 eq.) for 2-24 h at 20-60° C. until the starting material was completely converted into the carbamate. The reaction mixture was cooled to 15° C. and 3-(dimethylamino)propylamine (1.5 eq) was added. Stirring was continued for 30 min. The reaction mixture was extracted with 2M aq. HCl, water and aq. NaHCO₃, dried on MgSO₄ and evaporated to give the carbamate 35a, b and c, which was used without further purification. Compound 35a was obtained as a white solid foam. Yield=99%. Compound 35b was obtained as a transparent oil. Yield=93%. Compound 35c was obtained as a transparent oil. Yield=65%.

### Synthesis of Compounds 36a-b

Compounds 36a was synthesized in a similar manner as compound 35. Yield=99% Compounds 36b was synthesized in a similar manner as compound 35. Yield=65%

### Synthesis of Compounds 37a-c

To a solution of 34 or 35a or 35b (4.44 mmol, 1 eq.) in DCM (25 mL) was added paraformaldehyde (6.65 mmol, 1.5 eq.) followed by chlorotrimethylsilane (10.6 mmol, 2.4 eq.) and the resulting solution stirred at room temperature under an inert atmosphere of nitrogen till completion. The reaction mixture was concentrated under vacuum to give 37a to c. The compounds were used as such in the next step. Compound 37a was obtained as a colorless oil. Yield=93%. Compound 37b Yield=quantitative. Compound 37c was obtained as a white solid. Yield=quantitative.

### Synthesis of 38a-c

To a solution of 37a, b or c (8.5 mmol, 1 eq.) in DMF (20 mL) under an inert atmosphere of nitrogen was added potassium thioacetate (12.8 mmol, 1.5 eq.). The reaction mixture was stirred under N₂ at 50° C. till completion. The reaction mixture was diluted with EtOAc and washed with brine (3×). It was dissolved in DCM and purified by Flash chromatography affording 38a to c. Compound 38a was obtained as an oil. Yield=quantitative. LC-MS (ESI): r.t.=2.72 min, m/z calcd. for C19H₂₇NO₁₂S=493.1; found m/z=516.2 [M+Na]⁺. Compound 38b was obtained as an oil. Yield=73%. LC-MS (ESI): r.t.=2.89 min, m/z calcd. for C21H₃₁NO₁₂S=521.2; found m/z=544 [M+Na]⁺. Compound 38c was obtained as a colourless oil. Yield=88%. LC-MS (ESI): r.t.=2.71 min, m/z calcd. for C₂₁H₃₁NO₁₃S=537.2; found m/z=560 [M+Na]⁺.

### Synthesis of 39a-c

To a solution of 38a, b or c (1.1 mmol, 1 eq.) in MeOH (12 mL) was added NaOMe (3.3 mmol, 3 eq.). The reaction was stirred at room temperature under an inert atmosphere of nitrogen till completion. The reaction mixture was then quenched with Dowex H⁺ (pre-washed with water and MeOH), filtered and evaporated to dryness to give 39a to c. The product was used in the next step without further purification. Compound 39a was obtained as a colorless oil. Yield=quantitative. LC-MS (ESI): r.t.=0.34 min, m/z calcd. for C₉H₁₇NO₇S=283.1; found m/z=306.0 [M+Na]⁺, m/z=282.0 [M−H]⁻. Compound 39b. Yield=89%. LC-MS (ESI): r.t.=0.34 min, m/z calcd. for C₁H₂NO₇S=311.1; found m/z=334.0 [M+Na]⁺. Compound 39c was obtained as a colourless oil. Yield=98%. LC-MS (ESI): r.t.=0.54 min, m/z calcd. for C₁₁H₂₁NO₈S=327.1; found m/z=350.0 [M+Na]⁺.

### Synthesis of Compounds 40a-b

Compounds 40a was synthesized in a similar manner as compound 37. Yield=96%. Compound 40b was synthesized in a similar manner as compound 37. Yield=quantitative.

### Synthesis of Compound 41

Compound 41 was synthesized in a similar manner as compound 37. Yield=98%.

The below synthesis shows the formation of specific thiol compound 42b.

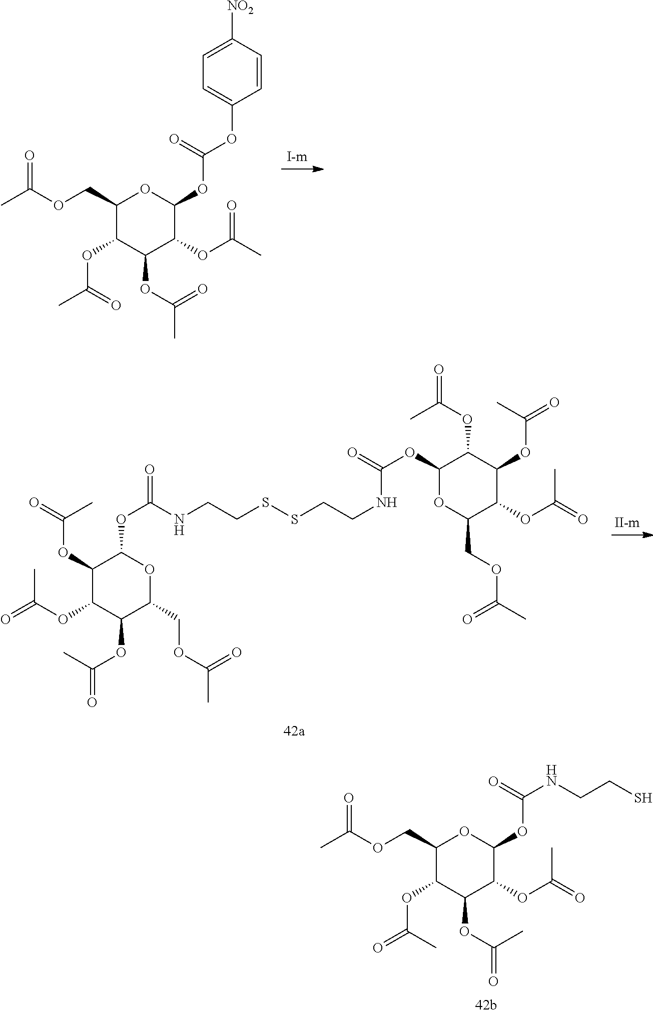 [SMILES: CC(=O)OC[C@H]1O[C@@H](OC(=O)Oc2ccc([N+](=O)[O-])cc2)[C@H](OC(C)=O)[C@@H](OC(C)=O)[C@@H]1OC(C)=O ; CC(=O)OC[C@H]1O[C@@H](OC(=O)NCCSSCCNC(=O)O[C@@H]2O[C@H](COC(C)=O)[C@@H](OC(C)=O)[C@H](OC(C)=O)[C@H]2OC(C)=O)[C@H](OC(C)=O)[C@@H](OC(C)=O)[C@@H]1OC(C)=O ; CC(=O)OC[C@H]1O[C@@H](OC(=O)NCCS)[C@H](OC(C)=O)[C@@H](OC(C)=O)[C@@H]1OC(C)=O]

### Synthesis of Compound 42a

To a solution of cystamine hydrochloride (439 mg, 1.95 mmol, 1 eq.) in DCM (48 mL) was added TEA (1.63 mL, 11.7 mmol, 6 eq.) followed by the p-nitrophenyl 2,3,4,6-tetra-O-acetyl-β-D-glucopyranosyl carbonate (2 g, 3.9 mmol, 2 eq.). The solution was stirred overnight at room temperature under an inert atmosphere of nitrogen. The reaction mixture was diluted with DCM and washed with brine. The organic layer was dried, concentrated and purified by flash chromatography yielding disulfide 42a (1.49 g, 1.65 mmol, 85%) as a white solid.

### Synthesis of Compound 42b

To compound 42a (150 mg, 0.17 mmol, 1 eq.) were added MeOH (0.9 mL) and DTT (81 mg, 0.51 mmol, 3 eq.). The reaction was stirred at room temperature until completion. The crude product was then concentrated and purified by column chromatography yielding compound 42b (quantitative) as a white solid.

The below synthesis shows the formation of specific thiol compound 48.

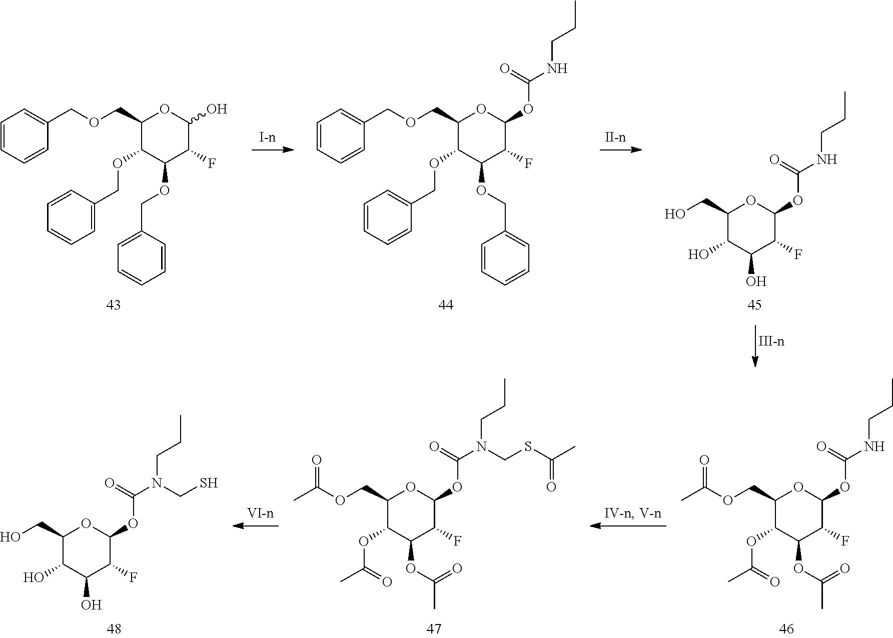 [SMILES: OC1O[C@H](COCc2ccccc2)[C@@H](OCc2ccccc2)[C@H](OCc2ccccc2)[C@H]1F ; CCCNC(=O)O[C@@H]1O[C@H](COCc2ccccc2)[C@@H](OCc2ccccc2)[C@H](OCc2ccccc2)[C@H]1F ; CCCNC(=O)O[C@@H]1O[C@H](CO)[C@@H](O)[C@H](O)[C@H]1F ; CCCN(CS)C(=O)O[C@@H]1O[C@H](CO)[C@@H](O)[C@H](O)[C@H]1F ; CCCN(CSC(C)=O)C(=O)O[C@@H]1O[C@H](COC(C)=O)[C@@H](OC(C)=O)[C@H](OC(C)=O)[C@H]1F ; CCCNC(=O)O[C@@H]1O[C@H](COC(C)=O)[C@@H](OC(C)=O)[C@H](OC(C)=O)[C@H]1F]

### Synthesis of β Pure Anomer 44

2-Fluoro-2-deoxy-3,4,6-tri-O-benzyl-D-glucopyranose 43 (6 g, 13.2 mmol, 1.0 eq.) (see publication European Journal of Organic Chemistry 5 (2012) 948-959), propyl isocyanate (4.69 mL, 49.6 mmol, 3.75 eq.) and TEA (3.68 mL, 26.4 mmol, 2 eq.) were dissolved in toluene (63 mL). The reaction was stirred at room temperature under an inert atmosphere of nitrogen for 45 h. 3-(Dimethylamino)-1-propylamine (4.57 mL, 36.3 mmol, 2.75 eq.) was then added to quench residual isocyanate. The reaction mixture was stirred for another 30 minutes. The mixture was diluted with EtOAc and washed with 1 M HCl, a saturated solution of NaHCO₃ and brine. The organic layer was dried using MgSO₄, filtered and concentrated in vacuo. The crude product was purified by recrystalization in EtOAc/Heptane yielding the β pure anomer 44 (6.7 g, 12.5 mmol, 95%) as a white crystalline solid. LC-MS (ESI): r.t.=3.67 min, m/z calcd. for C₃₁H₃₆FNO₆=537.3, found m/z=560.3 [M+Na]⁺.

### Synthesis of Compound 45

Carbamate 44 (5.8 g, 10.9 mmol, 1.0 equiv.) was dissolved in a mixture of MeOH (46.0 mL) and EtOAc (46.0 mL) and activated carbon on Pd (410.3 mg, 3.9 mmol, 0.4 eq.) was added. The mixture was then stirred under hydrogen atmosphere at 8 psi at room temperature for 3 days before being filtered through celite. The solvents were evaporated under reduced pressure and the crude product was purified using column chromatography (silica, 0→10% MeOH in DCM) to yield 45 (2.5 g, 9.3 mmol, 85%) as white solids. 19-F NMR (377 MHz; MeOD): δ −201.6, −201.8; LC-MS (ESI): r.t.=0.78 min, m/z calcd. for C10H18FNO6=267.1, found m/z=290 [M+Na]⁺.

### Synthesis of Compound 46

Carbamate 45 (2.5 g, 9.3 mmol, 1 equiv.) was dissolved in pyridine (38.0 mL) and DMAP (113.6 mg, 0.9 mmol, 0.1 equiv.) was added. Acetic anhydride (3.5 mL, 37.1 mmol, 4.0 equiv.) was added and the mixture was stirred for 3 hours under an inert atmosphere of nitrogen at room temperature. After reacting, the mixture was diluted with DCM and washed with a saturated solution of NaHCO₃ and brine. The organic layer was dried with MgSO₄ and the solvents were evaporated under reduced pressure. The crude product was purified using column chromatography (silica, 10→30% EtOAc in heptane) to yield 46 (3.8 g, 9.1 mmol, 98%) as a clear oil. 19-F NMR (377 MHz; CDCl3): δ −200.6 LC-MS (ESI): r.t.=2.65 min, m/z calcd. for C₁₆H₂₄FNO₉=393.1, found m/z=416.2 [M+Na]⁺.

### Synthesis of Compound 47

### Step IV-n)

To a solution of compound 46 (2.1 g, 5.34 mmol, 1 eq.) in DCM (46.2 mL) was added paraformaldehyde (240 mg, 8.01 mmol, 1.5 eq.) followed by chlorotrimethylsilane (5.08 mL, 40 mmol, 7.5 eq.) and the resulting solution stirred at room temperature under an inert atmosphere of nitrogen for 6 h. The reaction mixture was then evaporated to dryness and co-evaporated with DCM.

### Step V-n)

The crude intermediate obtained was dissolved in DMF (21.5 mL) under N₂ and potassium thioacetate (1.2 g, 10.7 mmol, 2 eq.) was added. The reaction mixture was stirred under an inert atmosphere of nitrogen at 50° C. for 1 h30. The reaction mixture was then diluted with EtOAc and washed with brine, dried over MgSO₄ and concentrated in vacuo. The crude product was purified by flash chromatography (Silica, 0→50% EtOAc in heptane) yielding 47 (2.52 g, 5.23 mmol, 98%) as a transparent oil. LC-MS (ESI): r.t.=2.96 min, m/z calcd. for C₁₉H₂₈FNO₁₀S=481.1, found m/z=504.2 [M+Na]⁺.

### Synthesis of Compound 48

Sugar 47 (181.5 mg, 0.4 mmol, 1 eq.) was dissolved in MeOH (4 mL) and sodium methoxide (61.6 mg, 1.1 mmol, 3 eq.) was added. The mixture was then stirred for 4 hours under an inert atmosphere of nitrogen at room temperature. After completion, the reaction mixture was neutralized with Dowex H⁺, filtered and concentrated yielding 48 (111.3 mg, 0.36 mmol, 93%) as a transparent oil. The product is used as such in the next step. LC-MS (ESI): r.t.=2.23 min, m/z calcd. for C₁₁H₂₀FNO₆S=313.1, found m/z=336.0 [M+Na]⁺.

The below schemes shows the preparation of specific thiol compounds 52a and 52b, respectively.

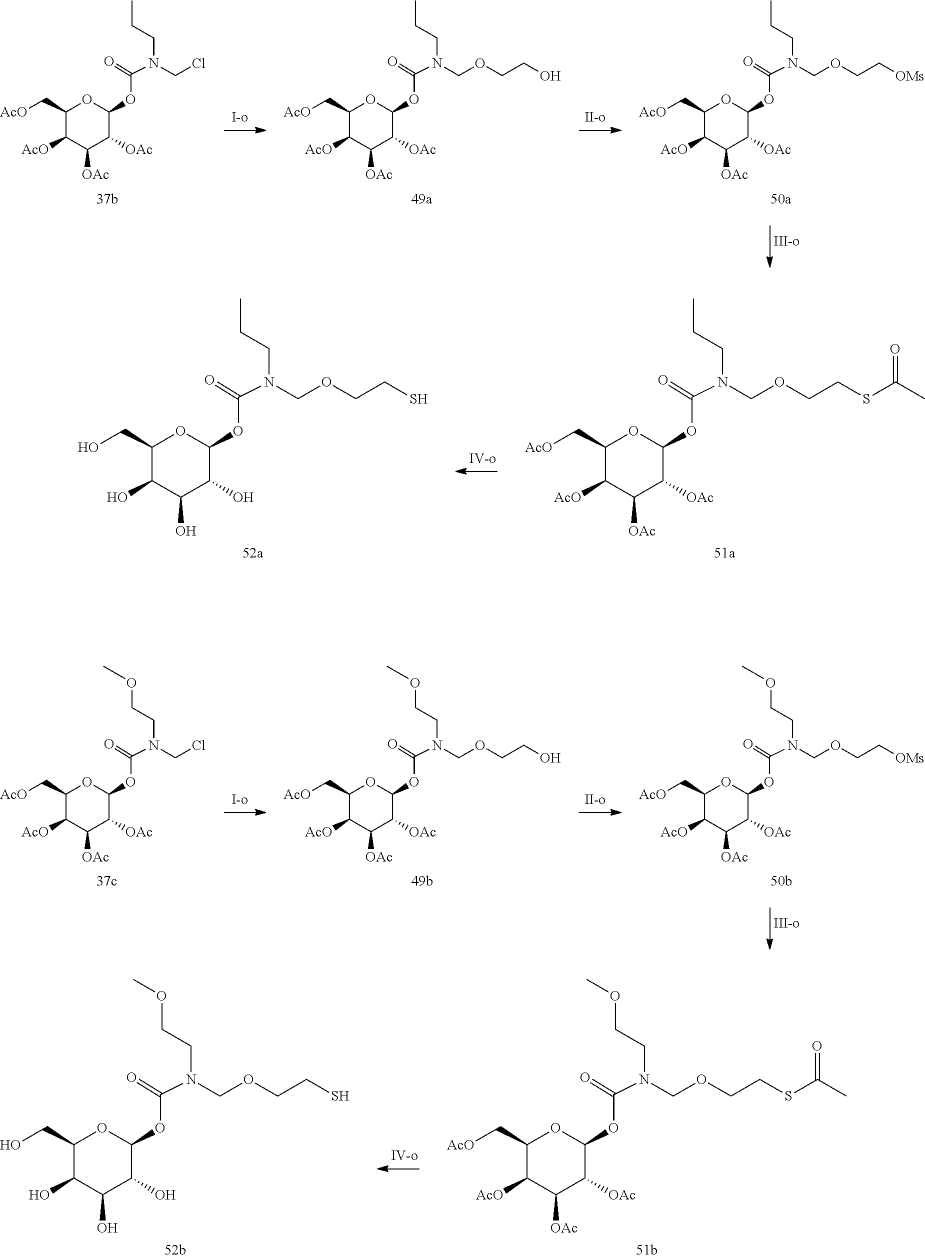

### Synthesis of Compounds 49a-b

To compound 37b or c (11.5 mmol, 1 eq.) dissolved in dry DCM (55 mL) was added ethyleneglycol (6.4 mL, 115 mmol, 10 eq.) followed by DIPEA (10 mL, 58 mmol, 5 eq.). The reaction mixture was stirred at room temperature till completion. The solution was diluted in EtOAc and washed with water, a 2 M solution of HCl and brine. The organic layer was dried over MgSO4, filtered and concentrated yielding 49a and b. Compound 49a was obtained as a white foam. Yield=66%. Compound 49b Yield=quantitative.

### Synthesis of Compounds 50a-b

To compound 49a or b (1.2 g, 2.2 mmol, 1 eq.) in pyridine (15 mL) at 0° C. was slowly added MsCl (0.3 mL, 4.4 mmol, 2 eq.) The reaction was then stirred at room temperature under an inert atmosphere of nitrogen until completion. The mixture was diluted in EtOAc, washed with water, a 2 M solution of HCl, a saturated solution of NaHCO₃ and brine. The organic layer was dried over MgSO₄, filtered and evaporated under reduced pressure yielding 50a and b. Compound 50a yield=quantitative. Compound 50b yield=97%.

### Synthesis of Compounds 51a-b

Compound 51a and b were obtained in a similar manner to that describe for compound 22. Compound 51a was obtained as an orange oil. Yield=92%. Compound 51b was obtained as a dark orange oil. Yield=60%.

### Synthesis of Compounds 52a-b

Compound 52a and b were obtained in a similar manner to that describe for compound 23. Compound 52a yield=quantitative. Compound 52b yield=quantitative.

The below schemes show the preparation of thiol compound 55.

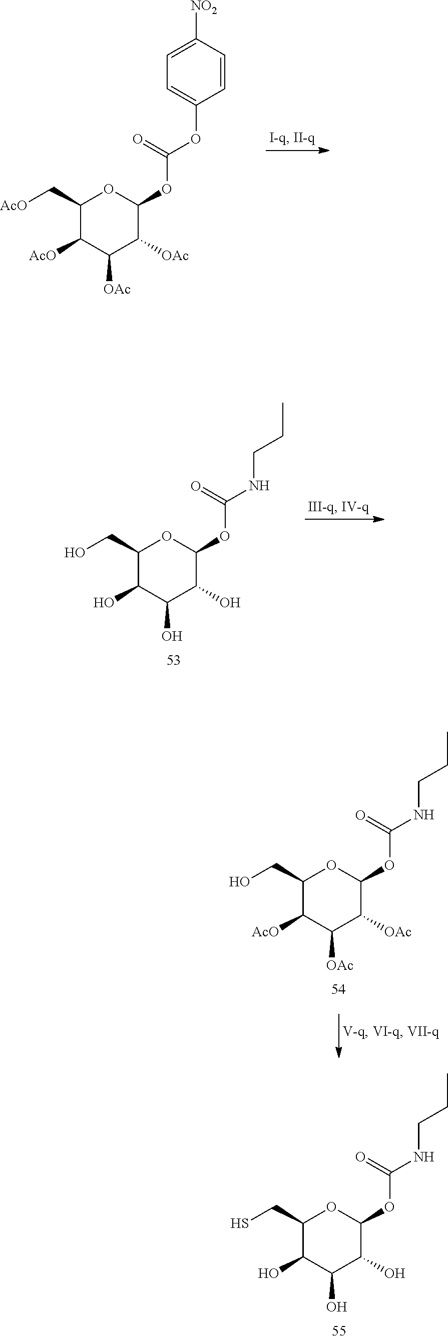 [SMILES: CC(=O)OC[C@H]1O[C@@H](OC(=O)Oc2ccc([N+](=O)[O-])cc2)[C@H](OC(C)=O)[C@@H](OC(C)=O)[C@H]1OC(C)=O ; CCCNC(=O)O[C@@H]1O[C@H](CO)[C@H](O)[C@H](O)[C@H]1O ; CCCNC(=O)O[C@@H]1O[C@H](CO)[C@H](OC(C)=O)[C@H](OC(C)=O)[C@H]1OC(C)=O ; CCCNC(=O)O[C@@H]1O[C@H](CS)[C@H](O)[C@H](O)[C@H]1O]

### Synthesis of Compound 53

### Step I-q)

p-Nitrophenyl 2,3,4,6-O-acetyl-β-D-glucopyranosyl carbonate (2 mM) was dissolved in DCM (10 mL). n-Propylamine (2.5 mM) and triethylamine (2 mM) were added, and the reaction mixture was stirred overnight. The mixture was diluted with DCM and extracted successively with 1 M HCl, water and aq. NaHCO₃ (2×), dried (MgSO₄) and concentrated. Purification of the residue by flash chromatography with heptane-ethyl acetate afforded the propyl carbamate (1.8 mM).

### Step II-q)

The carbamate (1.8 mM) was dissolved in methanol. Sodium methoxide (0.2 mM) was added, and the mixture was stirred for 1 h. Dowex H⁺ was added and the mixture was filtered and concentrated. The resulting unprotected propyl carbamate 53 was dried in vacuo and used without further purification.

### Synthesis of Compound 54

### Step III-q)

Carbamate 53 (1.8 mM) was dissolved in pyridine (10 mL), followed by the addition of TBS-Cl (2.7 mM). The mixture was stirred overnight. Acetic anhydride (8 mM) was added and stirring was continued for another 18 h. Water was added, and the mixture was concentrated. The residue was diluted with ethyl acetate, extracted successively with 1M HCl, water and aq. NaHCO₃, dried (MgSO₄) and concentrated. The resulting oil was purified by flash chromatography with heptane-ethyl acetate to give the acetylated 6-O-TBS derivative (1.35 mM).

### Step IV-q)

The product from step iii (1.35 mM) was dissolved in acetonitrile (10 mL). Water (1 mL) was added, followed by solid pTsOH (4 mM). The resulting reaction mixture was stirred for 1 h. Water was added, and the mixture was extracted with DCM. The organic layer was dried (MgSO₄) and concentrated. The residue was purified by flash chromatography with heptane-ethyl acetate to give the 6-hydroxy derivative 54 (1.1 mM). ¹H-NMR (400 MHz; CDCl₃): δ 3.17, dd, 1H, H-6a; 3.55, dd, 1H, H-6b; 5.67, d, 1H, H-1.

### Synthesis of Compound 55

### Step V-q)

To a solution of 54 in toluene was added successively imidazole (3.3 mM), PPh₃ (2.2 mM) and iodine (1.7 mM). The reaction mixture was heated at reflux for 2 h, then cooled and quenched with aq. NaHCO₃. The aqueous layer was extracted with ethyl acetate and the combined organic phases were treated with 12 until a brown color persisted. The organic phase was extracted with aq. Na₂S₂O₃, dried over Na₂SO₄, and concentrated under reduced pressure. The residue was purified by flash chromatography with heptane-ethyl acetate to give the 6-iodide (0.9 mM).

### Step VI-q)

The 6-iodide was treated with potassium thioacetate (2.7 mM) in DMF at 50° for 1 h. Ethyl acetate was added, and the mixture was extracted with brine (3×). The organic layer was dried (MgSO₄) and concentrated. The residue was purified by flash chromatography with heptane-ethyl acetate to give 6-thioacetate (0.75 mM).

### Step VII-q)

The thioacetate was dissolved in methanol. Sodium methoxide (1 mM) was added, and the reaction mixture was stirred for 1 h. Dowex H⁺ was added and the mixture was filtered and concentrated. The residue was dried under vacuo to give 6-mercapto derivative 55 (0.75 mM). LC-MS (ESI): r.t.=0.95 min, m/z calcd. for C₁₀H₁₉NO₆S=281.1, found m/z=304 [M+Na]⁺.

### Synthesis of Compound 56

Compound 56 was prepared in the same way as described for 55 but using p-nitrophenyl 2,3,4,6-tetra-O-acetyl-β-D-glucopyranosyl carbonate as the starting material and diethylamine as the amine. LCMS (ESI): r.t.=2.19 min, m/z calcd. for C11H₂₁NO₆S=295.1, found: m/z=318.2 [M+Na]⁺; m/z=294.0 [M−H]⁻.

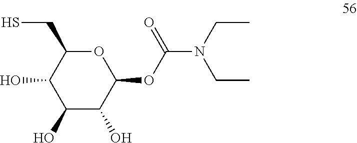 [SMILES: CCN(CC)C(=O)O[C@@H]1O[C@H](CS)[C@@H](O)[C@H](O)[C@H]1O]

### Synthesis of Compound 57

Compound 57 was prepared in the same way as described for 55 but using p-nitrophenyl 2,3,4,6-tetra-O-acetyl-β-D-glucopyranosyl carbonate as the starting material and N-methyl-N-propylamine as the amine. LCMS (ESI): r.t.=1.52 min, m/z calcd. for C₁₁H₂₁NO₆S=295.1; found: m/z=318.2 [M+Na]⁺; m/z=294.1 [M−H]⁻.

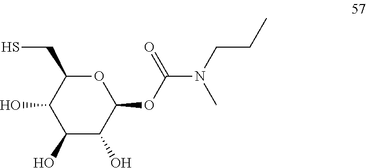 [SMILES: CCCN(C)C(=O)O[C@@H]1O[C@H](CS)[C@@H](O)[C@H](O)[C@H]1O]

### Synthesis of Compound 58

Compound 58 was prepared in the same way, but using p-nitrophenyl 2,3,4,6-tetra-O-acetyl-β-D-glucopyranosyl carbonate as the starting material. ¹H-NMR (400 MHz; CD₃OD): δ 5.32, d, 1H, H-1.

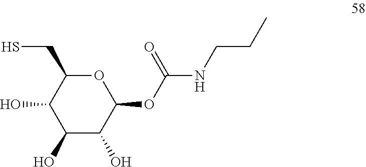 [SMILES: CCCNC(=O)O[C@@H]1O[C@H](CS)[C@@H](O)[C@H](O)[C@H]1O]

Coupling of Intermediate Thiosulfate-Drug Conjugates (Also Called Alkyl- or Aryl-Sulfonylsulfanylmethyl-Drug Conjugate) with Organic Moiety G[C]—SH

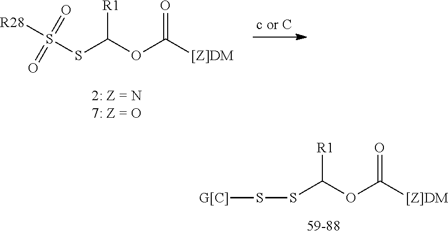

### First Protocol—Step C or Second Protocol—Step c

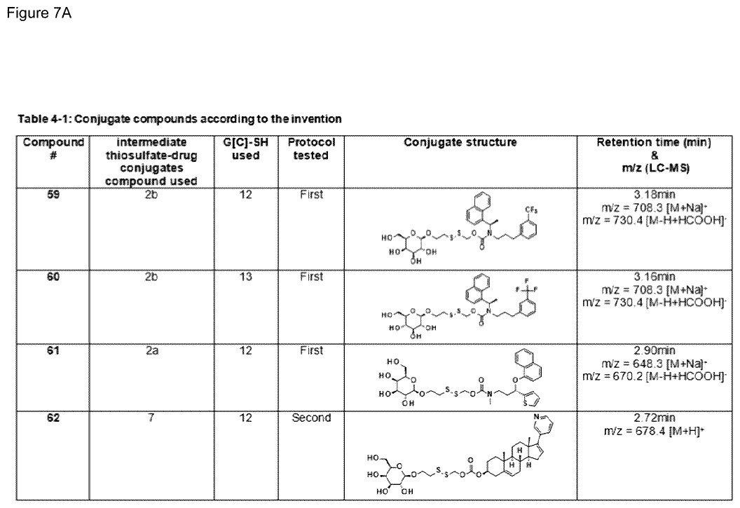

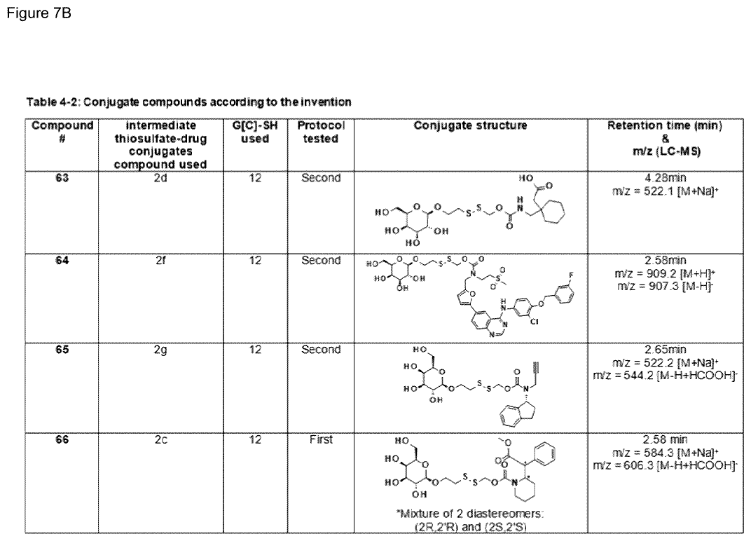

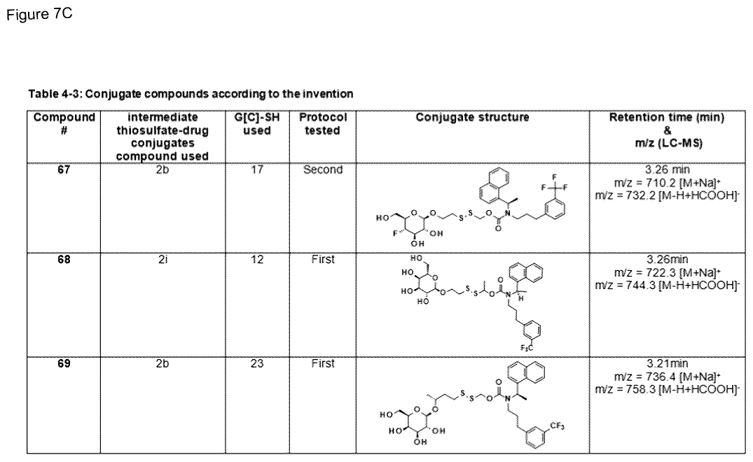

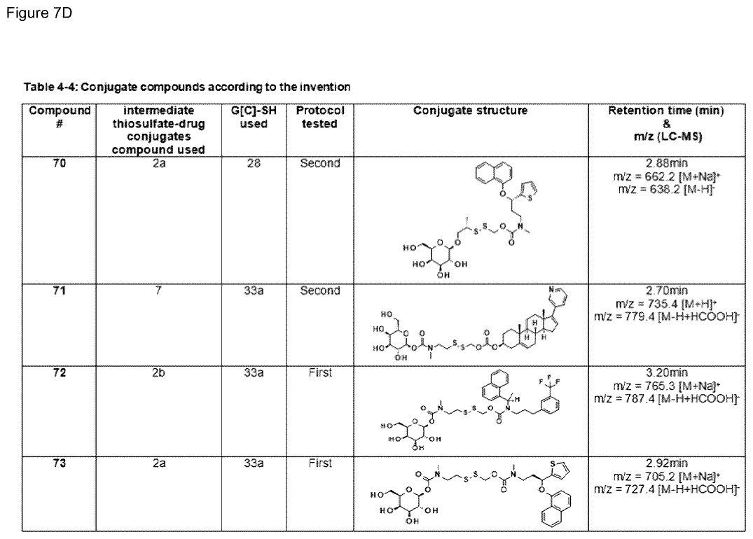

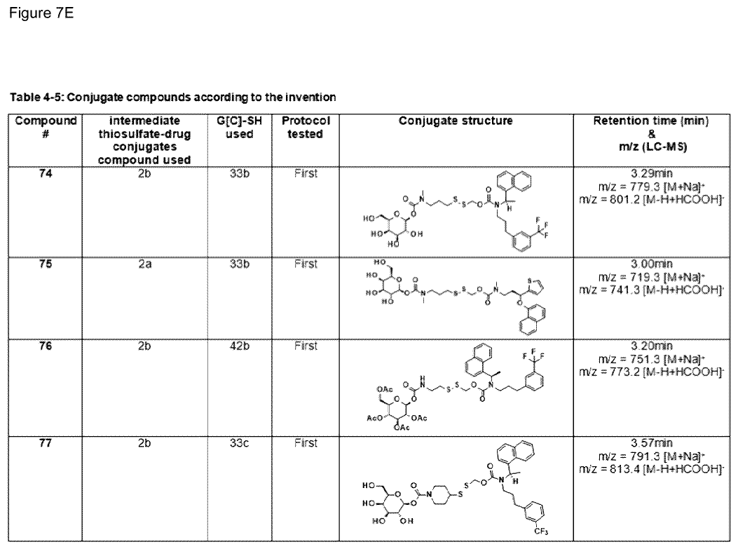

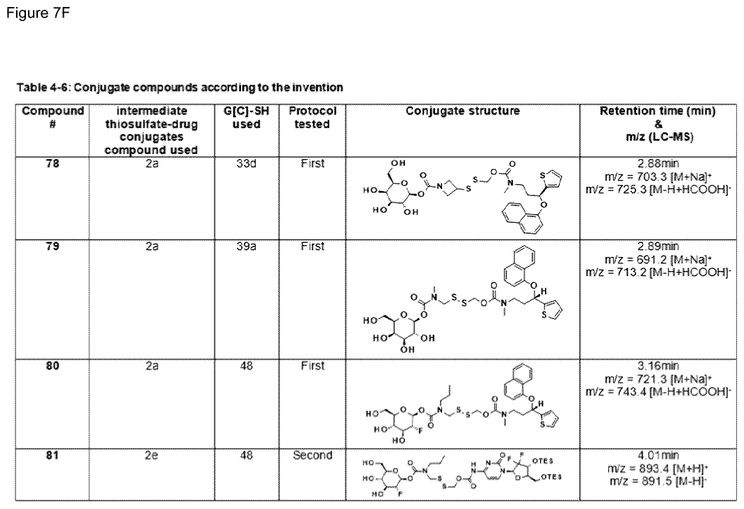

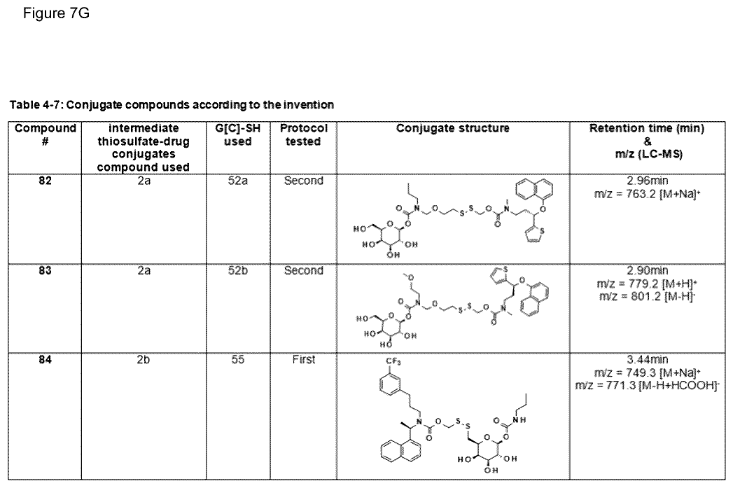

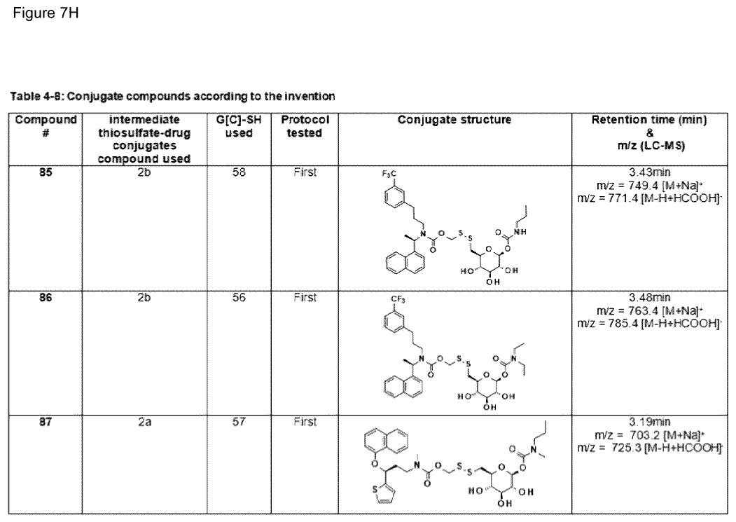

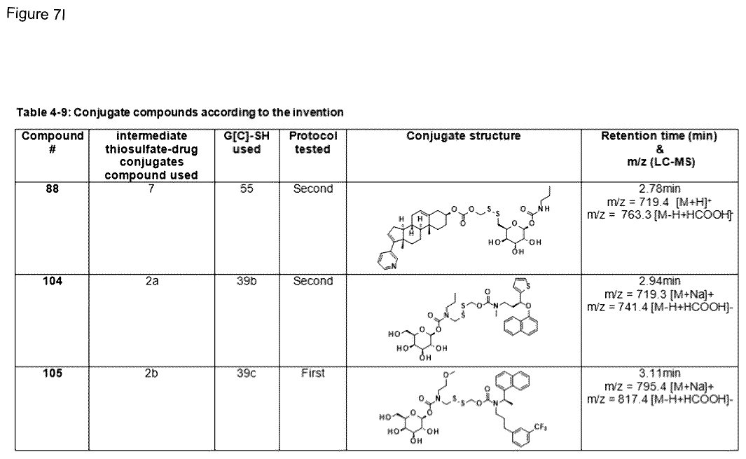

Several compounds according to the present invention (either according to Formula I or Ia) were prepared using step C of the First Protocol or step c of the Second Protocol. The compounds are disclosed in Table 4 below as compounds 59-88, 104 and 105. Tables 4 in FIGS. 7A-I show the compounds 59-88, 104 and 105 that have been prepared. Table 4-1: compounds 59-62/Table 4-2: compounds 63-66 ITable 4-3: compounds 67-69/Table 4-4: compounds 70-73/Table 4-5: compounds 74-77/Table 4-6: compounds 78-81 ITable 4-7: compounds 82-84/Table 4-8: compounds 85-87/Table 4-9: compounds 88, 104 and 105. It should be noted that compounds 76 and 81 still comprise protective groups, acetate (Ac) and triethoxysilane (TES) respectively. The step of deprotection thereof is shown below. The compounds of Tables 4-1 to 4-7 comprise a group G that is according to Formula V, with the exception of compound 84 in Table 4-7 which comprises a group G that falls under Formula VI and VII. The compounds of Tables 4-8 and 4-9 comprise a group G that is according to Formulas VI and VII, with the exception of compounds 104 and 108 in Table 4-8 which comprises a group G that falls under Formula V.

### Step of Deprotection of Hydroxyl Groups in Drug Moiety

### Second Protocol—Step e

The below synthesis is one specific example (going from compound 81 to compound 89) of an optional step in the synthesis of a compound according to Formula I or Ia. The step e can be present as an optional step in claim 11, after step c or after step d.

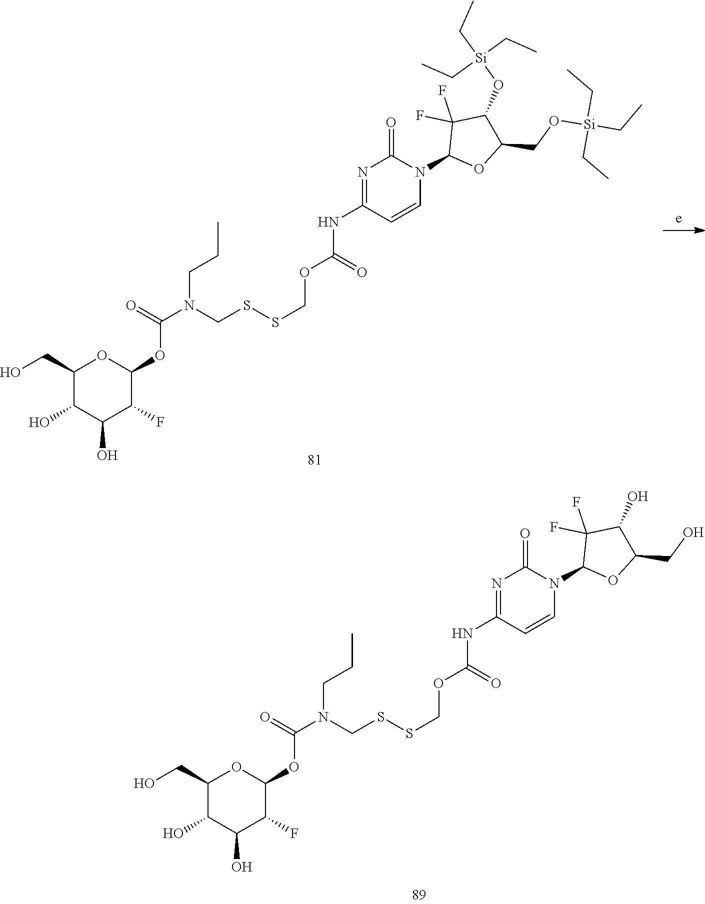

### Synthesis of Compound 89

To conjugate 81 (382 mg, 0.43 mmol, 1 eq.) dissolved in MeOH (8 mL) was added a 1.25 M solution of HCl in MeOH (0.684 mL, 2 eq.). The reaction mixture was stirred at room temperature for 20 min. The mixture was concentrated and precipitated from a mixture of MeOH and Et₂O. The precipitate was filtered and purified by reverse phase column chromatography (RP silica, water/ACN 95/5→0/100) yielding the desired compound 89 (225 mg, 0.34 mmol, 79%). LC-MS (ESI): r.t.=2.39 min, m/z calcd. For C22H₃₁F₃N₄O₁₂S₂=664.1; found m/z=665.2 [M+H]⁺, m/z=663.3 [M−H]⁻.

### Step of Deprotection of Hydroxyl Groups in G[C] Moiety

### First Protocol—Step D

The below synthesis is one specific example (going from compound 76 to compound 90) of an optional step in the synthesis of a compound according to Formula I or Ia. The step D can be present as an optional step in claim 12, after step C.

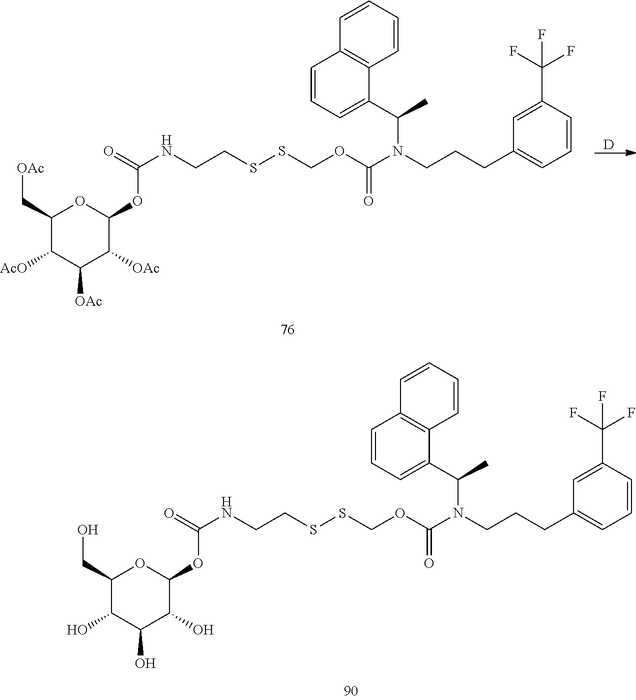

### Synthesis of Compound 90

A solution of NaOMe (10 mg, 0.2 mmol, 0.5 eq.) in MeOH (5.5 mL) was added to 76 (343 mg, 0.38 mmol, 1 eq.). The resulting reaction mixture was stirred at room temperature for 20 min. The reaction mixture was neutralized with Amberlite CG 50 type 1, filtered and concentrated. The crude product was purified by flash chromatography (silica, 0→15% DCM in MeOH) to give 90 (181 mg, 0.25 mmol, 65%) as a white solid. LC-MS (ESI): r.t.=3.20 min, m/z calcd. For C33H₃₉F₃N₂O₉S₂=728.2; found m/z=751.3 [M+Na]⁺, m/z=773.2 [M−H+HCOOH]⁻.

### Modification of G[C]-Moiety in Conjugate Compound

The synthesis below shows the modification of compound 8h according to the invention in order to obtain compounds 92a or 92b according to the invention.

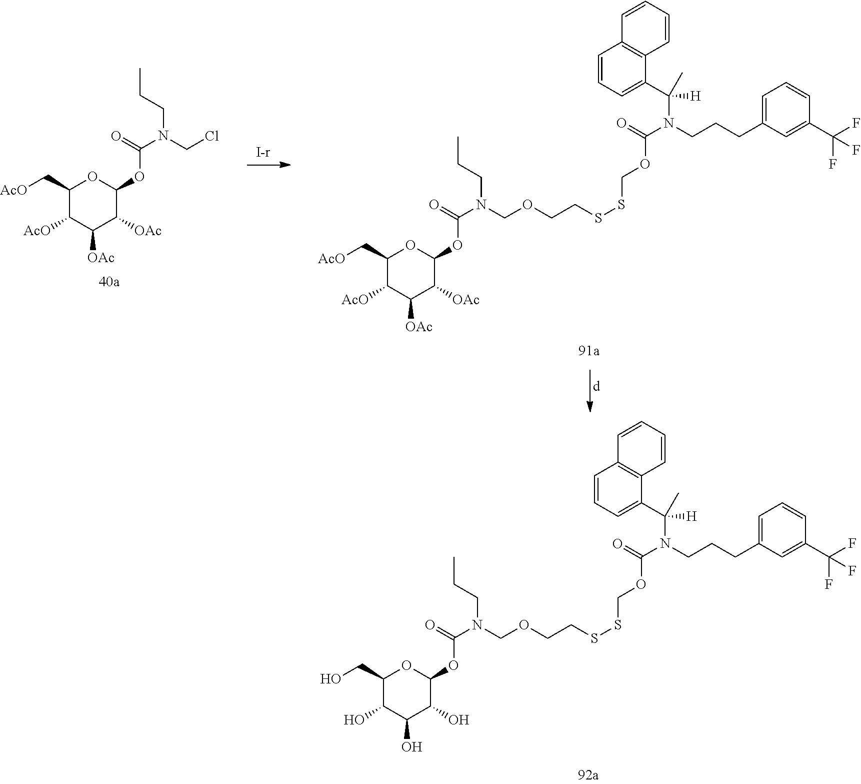 [SMILES: CCCN(CCl)C(=O)O[C@@H]1O[C@H](COC(C)=O)[C@@H](OC(C)=O)[C@H](OC(C)=O)[C@H]1OC(C)=O ; CCCN(COCCSSCOC(=O)N(CCCc1cccc(C(F)(F)F)c1)[C@H](C)c1cccc2ccccc12)C(=O)O[C@@H]1O[C@H](COC(C)=O)[C@@H](OC(C)=O)[C@H](OC(C)=O)[C@H]1OC(C)=O ; CCCN(COCCSSCOC(=O)N(CCCc1cccc(C(F)(F)F)c1)[C@H](C)c1cccc2ccccc12)C(=O)O[C@@H]1O[C@H](CO)[C@@H](O)[C@H](O)[C@H]1O]

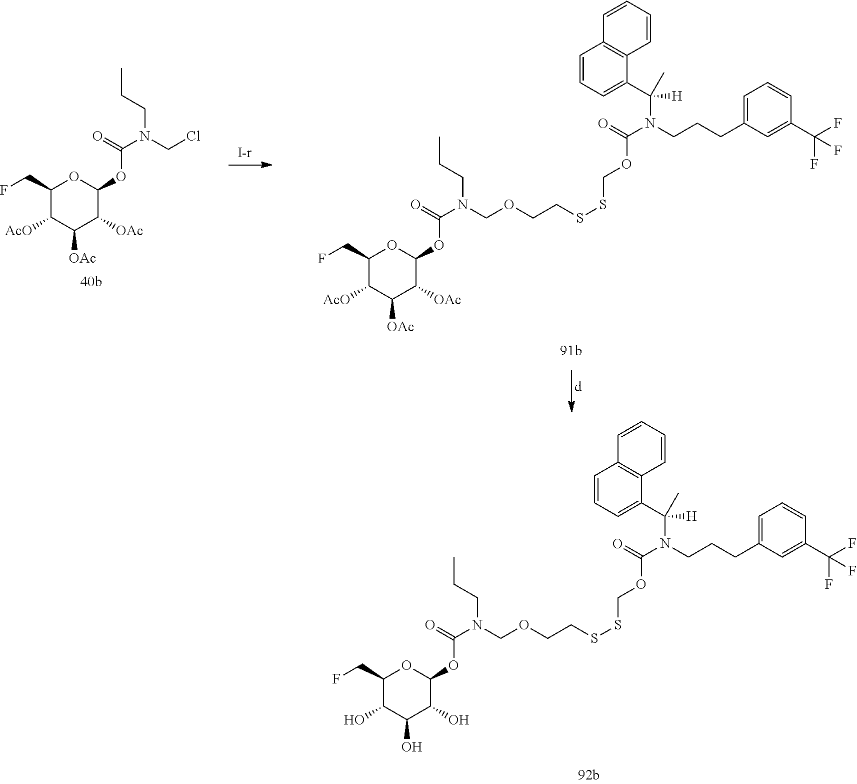 [SMILES: CCCN(CCl)C(=O)O[C@@H]1O[C@H](CF)[C@@H](OC(C)=O)[C@H](OC(C)=O)[C@H]1OC(C)=O ; CCCN(COCCSSCOC(=O)N(CCCc1cccc(C(F)(F)F)c1)[C@H](C)c1cccc2ccccc12)C(=O)O[C@@H]1O[C@H](CF)[C@@H](OC(C)=O)[C@H](OC(C)=O)[C@H]1OC(C)=O ; CCCN(COCCSSCOC(=O)N(CCCc1cccc(C(F)(F)F)c1)[C@H](C)c1cccc2ccccc12)C(=O)O[C@@H]1O[C@H](CF)[C@@H](O)[C@H](O)[C@H]1O]

### Synthesis of Compounds 91a-b

To a solution of 40a or b (1.4 mmol, 2 eq.) and compound 8h (0.7 mmol, 1 eq.) in dry DCM (4 mL) was added DIPEA (2.6 mmol, 4 eq.). The reaction was stirred at room temperature under an inert atmosphere of nitrogen until completion. The reaction mixture was diluted with DCM, washed with brine, dried over MgSO₄, filtered and concentrated. The crude product was purified by flash chromatography yielding 91a or b. Compound 91a Yield=87%. LC-MS (ESI): r.t.=4.29 min, m/z calcd. For C45H₅₅F₃N₂O₁₄S₂=968.3; found m/z=991.4 [M+Na]⁺. Compound 91b Yield=67%.

### Synthesis of Compounds 92a-b

This is a deprotection step according to d or D as shown above. A solution of NaOMe (0.2 mmol, 0.5 eq.) in MeOH (5 mL) was added to compound 91a or b (0.4 mmol, 1 eq.). The resulting reaction mixture was stirred at room temperature for 20 min. The reaction mixture was neutralized with Amberlite CG 50 type 1, filtered and concentrated to give 92a or b. Compound 92a was obtained as a white solid. Yield=94%. LC-MS (ESI): r.t.=3.42 min, m/z calcd. For C₃₇H₄₇F₃N₂O₁₀S₂=800.3; found m/z=823.4 [M+Na]⁺, m/z=845.3 [M−H+HCOOH]⁻. Compound 92b was obtained as a white solid. Yield=64%. LC-MS (ESI): r.t.=3.64 min, m/z calcd. For C₃₇H₄₆F₄N₂O₉S₂=802.3; found m/z=825.4 [M+Na]⁺, m/z=847.4 [M−H+HCOOH]⁻.

A similar modification of compound 8h according to the invention in order to obtain compounds 93a or 93b according to the invention was carried out.

### Synthesis of Compounds 93a-b

Compound 93a (with Cinacalcet) was synthesized in a similar manner as conjugate 92 starting from compounds 8h and 37b. LC-MS (ESI): r.t.=3.40 min, m/z calcd. For C37H₄₇F₃N₂O₁₀S₂=800.3; found m/z=823.3 [M+Na]⁺, m/z=845.4 [M−H+HCOOH]⁻.

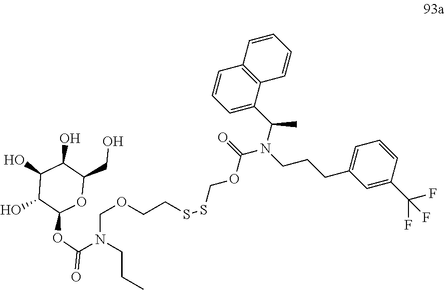 [SMILES: CCCN(COCCSSCOC(=O)N(CCCc1cccc(C(F)(F)F)c1)[C@H](C)c1cccc2ccccc12)C(=O)O[C@@H]1O[C@H](CO)[C@H](O)[C@H](O)[C@H]1O]

Compound 93b (with Duloxetine) was synthesized in a similar manner as conjugate 92 starting from compounds 8g and 41. LC-MS (ESI): r.t.=3.17 min, m/z calcd. For C₃₃H₄₃FN₂O₁₀S₃=742.2; found m/z=765.2 [M+Na]⁺, m/z=741.4 [M−H]⁻.

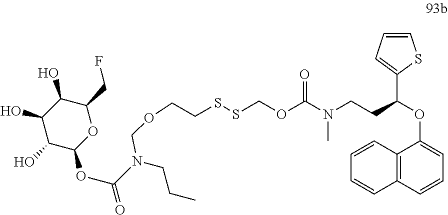 [SMILES: CCCN(COCCSSCOC(=O)N(C)CC[C@H](Oc1cccc2ccccc12)c1cccs1)C(=O)O[C@@H]1O[C@H](CF)[C@H](O)[C@H](O)[C@H]1O]

The synthesis below shows the modification of compound according to the invention 8i to obtain compound according to the invention 96

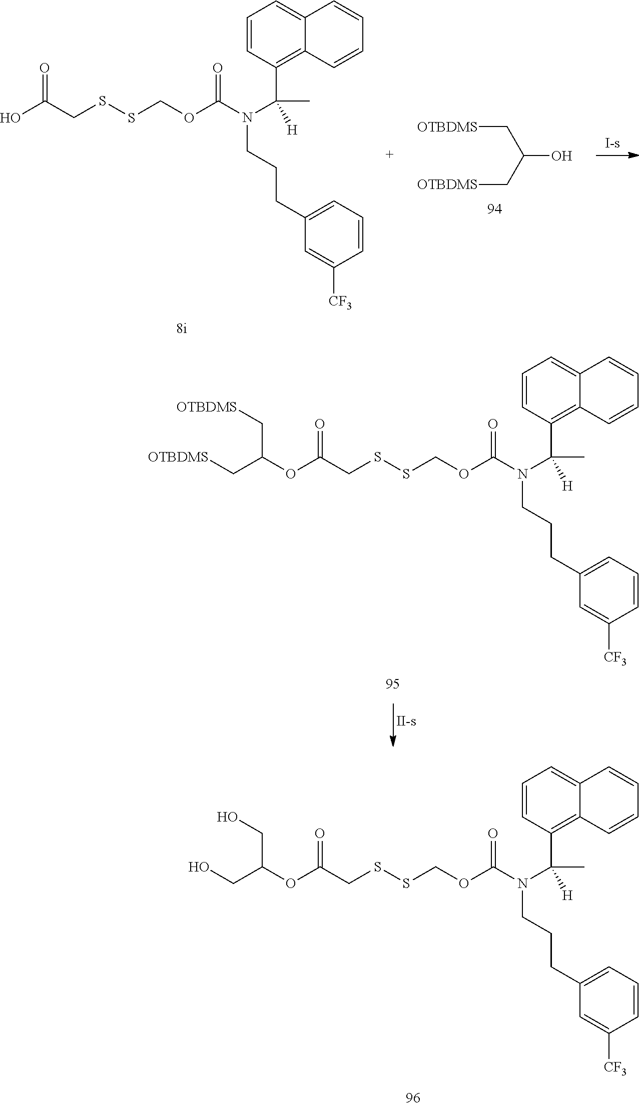 [SMILES: C[C@H](c1cccc2ccccc12)N(CCCc1cccc(C(F)(F)F)c1)C(=O)OCSSCC(=O)O ; CC(C)(C)[Si](C)(C)OCC(O)CO[Si](C)(C)C(C)(C)C ; C[C@H](c1cccc2ccccc12)N(CCCc1cccc(C(F)(F)F)c1)C(=O)OCSSCC(=O)OC(CO[Si](C)(C)C(C)(C)C)CO[Si](C)(C)C(C)(C)C ; C[C@H](c1cccc2ccccc12)N(CCCc1cccc(C(F)(F)F)c1)C(=O)OCSSCC(=O)OC(CO)CO]

### Compound 94

This compound 1,3-bis[[tert-butyl(dimethyl)silyl]oxy]propan-2-ol was obtained commercially.

### Synthesis of Compound 95

To a solution of 8i (0.780 g, 1.45 mmol) and 94 (0.698 g, 2.18 mmol) in THF (30.0 ml) was added triphenylphosphine (0.419 g, 1.60 mmol) and DIAD (0.587 g, 2.90 mmol). The reaction mixture was stirred at room temperature for 2 h. The mixture was diluted with EtOAc, washed with brine and purified by flash chromatography (silica, 0→10% EtOAc in heptane) to obtain 95 (0.880, 1.05 mmol, 72%) as a colorless oil. TLC: (EtOAc: hept, 40:60, v/v) R_{f}=0.76.

### Synthesis of Compound 96

To a solution of 95 (0.880 g, 1.05 mmol) in THF and H₂O (46.5 ml, 20:1, v/v) was added PTSA (0.398 g, 2.09 mmol). The reaction mixture was stirred overnight at room temperature, washed with a saturated solution of NaHCO₃ and concentrated in vacuo to obtain 96 (0.480 g, 0.785 mmol, 75.0%) as a colorless oil.

TLC: (EtOAc: heptane, 30:70, v/v) R_{f}=0.51.

The synthesis below shows the modification of compound according to the invention 8i to obtain compound according to the invention 98

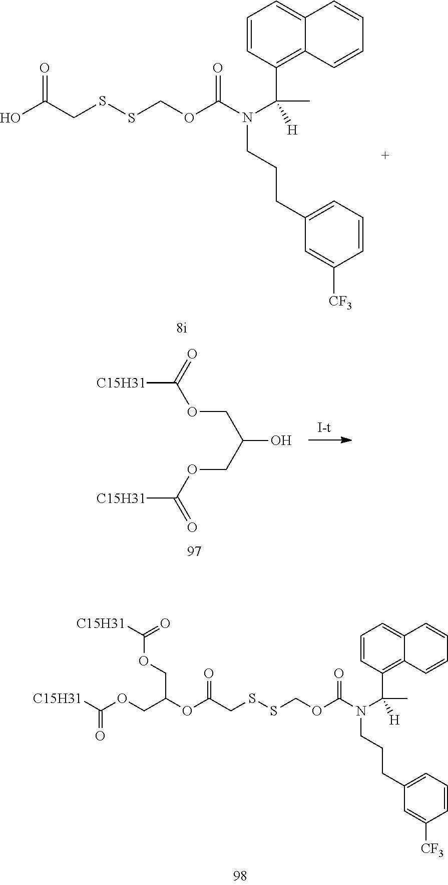

### Compound 97

This compound Glyceryl 1,3-dipalmitate was obtained commercially.

### Synthesis of Compound 98

To a stirring solution of 8i (0.850 g, 1.58 mmol) and 97 (0.900, 1.58 mmol) in THF (28.0 ml) was added triphenylphosphine (0.457 g, 1.74 mmol) and DIAD (0.639 g, 3.16 mmol). The reaction mixture was stirred at room temperature for 1 h, diluted with EtOAc, washed with H₂O, purified by flash chromatography (0→15% EtOAc in heptane) and concentrated in vacuo to obtain 98 (0.630 g, 0.579 mmol, 37%) as a colourless oil.

TLC: (EtOAc: heptane, 10:90, v/v) R_{f}=0.10.

The synthesis below shows the synthesis of the 2-disulfanylethyl compounds 102, 103 and 106 used as comparative compounds to support the present invention.

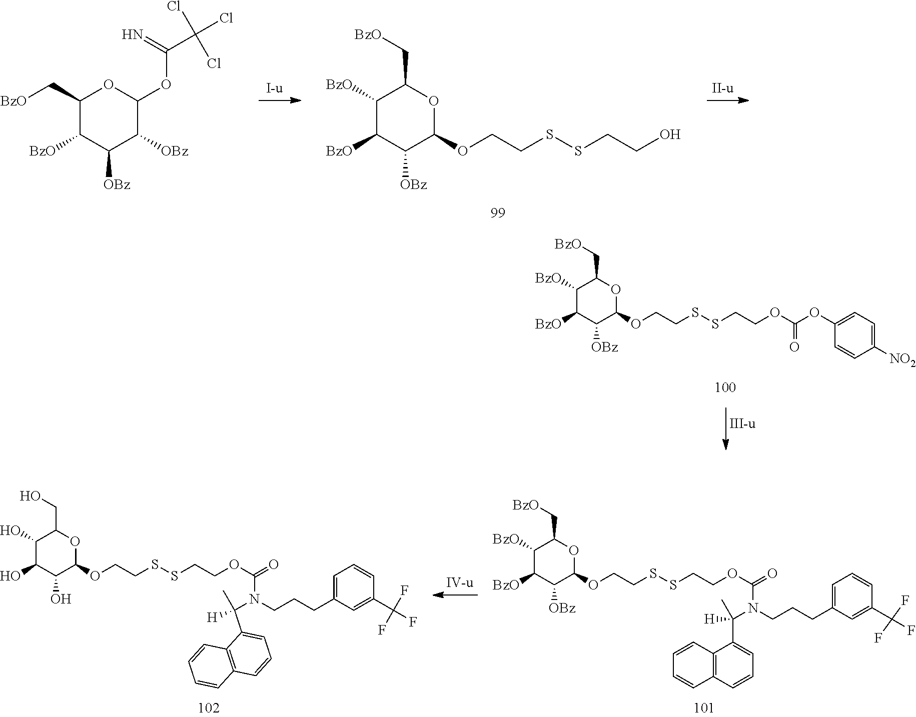

### Synthesis of Compound 99

To a solution of 2,3,4,6-tetra-O-benzoyl-D-glucopyranosyl trichloroacetimidate (3.9 g, 5.3 mmol, 1 eq.) and bis(2-hydroxyethyl) disulfide (3.25 mL, 26.6 mmol, 4 eq.) in dry DCM, molecular sieves in powdered form (100 mg) and BF₃·O(C₂H₅)₂ (65.6 μL, 532 mmol, 0.1 eq.) were added. The reaction mixture was stirred for 2 h at room temperature. The reaction was then quenched by addition of TEA, filtered concentrated in vacuo and purified by flash chromatography yielding compound 99 (2.4 g, 3.3 mmol, 62%).

LC-MS (ESI): r.t.=2.63 min, m/z calcd. For C₃₈H₃₆O₁₁S₂=732.2; found m/z=755.0 [M+Na]⁺.

### Synthesis of Compound 100

To a solution compound 99 (2.4 g, 3.3 mmol, 1 eq.) in DCM (10 mL) was added pyridine (662 mL, 8.2 mmol, 2.5 eq.) followed by 4-nitrophenyl chloroformate (825 mg, 4.1 mmol, 1.25 eq.). The reaction was stirred at room temperature under an inert atmosphere of nitrogen till completion. The reaction mixture was diluted with DCM and washed with a 1 M solution of HCl, a saturated solution of NaHCO₃ and brine. It was dried over Na₂SO₄, filtered and evaporated to dryness yielding compound 100 (quantitative). The product 100 was used as such in the next step.

### Synthesis of Compound 101

To a solution of compound 100 (900 mg, 1.0 mmol, 1 eq.) and Cinacalcet hydrochloride (790 mg, 2.0 mmol, 2 eq.) in DCM (20.0 ml), was added HOBT (153 mg, 1.0 mmol, 1 eq.) followed by TEA (419 μL, 3.0 mmol, 3 eq.). The reaction mixture was stirred at room temperature for 18 h. After this time, the solution was concentrated and purified by flash chromatography yielding 101 (1.12 g, 1.0 mmol, quantitative).

### Synthesis of Compound 102

To a solution of 101 (1.12 g, 1.0 mmol, 1 eq.) in MeOH (5 ml) and 1,4-Dioxane (5 ml) at room temperature was added NaOMe (27.1 mg, 0.50 mmol, 0.5 eq.). The reaction mixture was stirred until completion. The solution was neutralized with Dowex H⁺, filtered and concentrated. The crude was purified by flash chromatography to give compound 102 (195 mg, 0.28 mmol, 28%). LC-MS (ESI): r.t.=2.19 min, m/z calcd. For C33H₄₀F₃NO₈S₂=699.2; found m/z=722.2 [M+Na]⁺, m/z=744.2 [M−H+HCOOH]⁻.

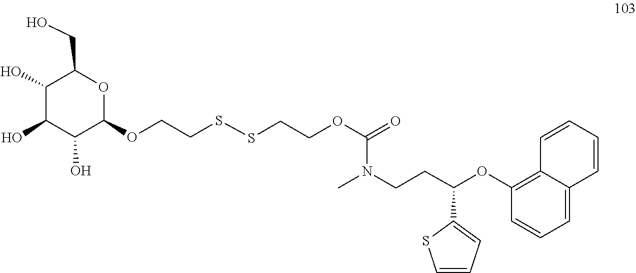 [SMILES: CN(CC[C@H](Oc1cccc2ccccc12)c1cccs1)C(=O)OCCSSCCO[C@@H]1O[C@H](CO)[C@@H](O)[C@H](O)[C@H]1O]

### Synthesis of Compound 103

Compound 103 was prepared in the same way as described for 102 but using Duloxetine as starting material. LCMS (ESI): r.t.=2.98 min, m/z calcd. for C29H₃₇NO₉S₃=639.2; found: m/z=662.0 [M+Na]⁺; m/z=684.0 [M−H+HCOOH]⁻.

While specific embodiments of the subject disclosure have been discussed, the above specification is illustrative and not restrictive. Many variations of the systems and methods herein will become apparent to those skilled in the art upon review of this specification. The full scope of the claimed systems and methods should be determined by reference to the claims, along with their full scope of equivalents, and the specification, along with such variations. Without wishing to be bound by any theory, it is believed that the results of the present invention are based on the use of linker moieties to improve the uptake and to achieve a more predictable hydrolysis rate of the drug glycosides. These linker moieties are positioned between the anomeric hydroxyl of the sugar residue and the drug and serve as molecular interface that create a certain distance between the sugar and drug moieties which may facilitate absorption and improve the interaction with an appropriate glycosidase. A self-immolative linker could prevent accumulation of intermediates. In a comparative experiment (results not shown) several self-immolative linkers such as diaminoethyl linker conjugates of Kalydeco and Abiraterone were prepared. Enzymatic removal of the glucose moiety of those conjugates did not result in formation of Kalydeco or Abiraterone, respectively. Rather, the intermediate aminoethyl conjugates were observed. Similar results were obtained with the glutathione-sensitive disulfanylethyl glycoconjugate of Abiraterone. Cleavage of the disulfide bond with glutathione did not produce significant amounts of Abiraterone, but rather produced the mercaptoethyl conjugate as well as various adducts. In contrast, compounds such as 7c, 7k and 17 were readily converted to Abiraterone and Kalydeco, respectively, upon treatment with b-glucosidase. These results indicate that while physicochemical characteristics of a drug can be improved by converting a drug into a drug-glycoside, significant improvement of oral bioavailability with this type of prodrug is not always achieved, contrary to the results of the invention as shown above.

1. A compound according to Formula I or a pharmaceutically acceptable salt thereof: 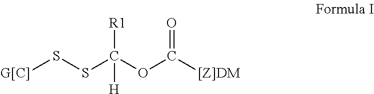
wherein each solid line represents a covalent bond, wherein H is hydrogen, O is oxygen, C is carbon, S is sulfur, and C═O is a carbonyl group;
wherein R1 is selected from the group consisting of hydrogen, methyl, ethyl, n-propyl, isopropyl, n-butyl, sec-butyl, and t-butyl, preferably R1 is hydrogen or methyl;
wherein G is an organic structure and [C] represents a carbon atom of G, preferably wherein G[C] is selected from a covalently bound group of saturated and unsaturated, cyclic and noncyclic, aromatic and non-aromatic organic structures containing C and H atoms and optionally containing one or more N, O, F, Cl, Br, I, B, P, and S atoms with the provision that the disulfide is always covalently attached to a primary, secondary or tertiary carbon atom C in G, and preferably with a further provision that this particular carbon atom C does not contain an OH, SH or NH group, a double bonded oxygen or a double bonded sulfur;
wherein DM is a drug moiety and [Z] represents a part of DM and is selected from the group consisting of O, S, and N.

2. The compound according to claim 1, wherein [Z] is selected from the group consisting of:
*O, to form 0-C representing an oxygen and a carbon atom of DM in which O is covalently bound to the carbonyl group of the compound of Formula I and wherein C is covalently bound to O and to three hydrogen atoms and/or carbon atoms of DM;
*N, to form N—C representing a nitrogen and a carbon atom of DM in which N is covalently bound to the carbonyl group of the compound of Formula I and wherein N and C are covalently bound to each other and to respectively one and three hydrogen atoms and/or carbon atoms of DM;
*S, to form S—C representing a sulfur and a carbon atom of DM in which S is covalently bound to the carbonyl group of the compound of Formula I and wherein C is covalently bound to S and to three hydrogen atoms and/or carbon atoms of DM; and
*O, to form O—N representing an oxygen and a nitrogen atom of DM in which O is covalently bound to the carbonyl group of the compound of Formula I and wherein N is covalently bound to O and to two hydrogen atoms and/or carbon atoms of DM.

3. The compound according to claim 1, wherein G[C] is represented by Formula IIa: 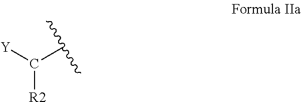
wherein Y is selected from the group consisting of compounds according to Formulas IIIa, IIIb, IIIc, IIId, and IIIe below: 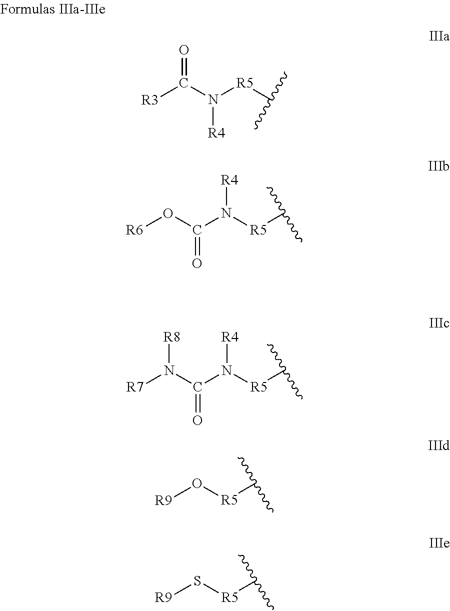
wherein R2 is hydrogen or methyl;
wherein R3, R6, and R9 are each independently a C1-20 (hetero)alkyl or a saturated or unsaturated 3-8 membered (hetero)cyclic structure;
wherein R4 is a hydrogen or a C1-6 (hetero)alkyl;
wherein R5 is selected from the group consisting of a bond, a C1-8 (hetero)alkyl, C1-8 (hetero)alkenyl, C1-8 (hetero)alkynyl, and a saturated or unsaturated 3-8 membered (hetero)cyclic structure; and
wherein R7 and R8 are independently selected from the group consisting of hydrogen, a C1-20 (hetero)alkyl, C1-20 (hetero)alkenyl, C1-20 (hetero)alkynyl, and a saturated or unsaturated 3-8 membered (hetero)cyclic structure; or
wherein G[C] is represented by Formula IIb: 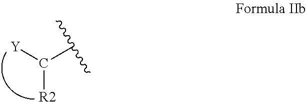
wherein Y and R2 together form a saturated or unsaturated 3-8 membered (hetero)cyclic structure.

4. The compound according to claim 1, wherein G[C] is represented by Formula IV: 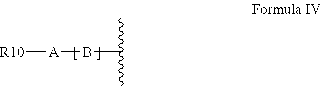
wherein R10 is selected from the group consisting of a carboxylate, hydroxyl, phosphate, phosphonate, sulfate, sulfonate, R11N(R12)-, NH₂CH(R13)C(═O)NH—, a 3-6 membered (hetero)cyclic ring and a sugar;
wherein A is selected from the group consisting of a bond, —CH₂—, —CH(NH₂)—, —CH₂CH₂—, —C(CH₂OH)H—, —CH₂CH(OH)—, and —C(═O)NH—;
wherein B is selected from the group consisting of —CH₂—, —O—CH₂—, —CH₂CH₂—O—, and —O—CH₂CH₂—;
wherein n is an integer from 1-20;
wherein R11 and R12 are independently selected from the group consisting of hydrogen, a C1-20 (hetero)alkyl, C1-20 (hetero)alkenyl, C1-20 (hetero)alkynyl, and a saturated or unsaturated 3-8 membered (hetero)cyclic structure; and
wherein R13 is selected from the group consisting of hydrogen, methyl, ethyl, propyl, isopropyl, sec-butyl, isobutyl, benzyl, 4-hydroxybenzyl, 2-methylthioethyl, hydroxymethyl, 4-aminobutyl, 3-aminopropyl, —CH₂—CH₂—CO—NH₂, —CH₂—CO—NH₂, —CH₂—CH₂—COOH, —CH₂—COOH, —CH₂—CH₂—CH₂—HN—(HN)═C(NH₂), and —CH₂-cycl(C═CH—N═CH—NH);
preferably wherein G[C] is selected from the group consisting of the following structures:

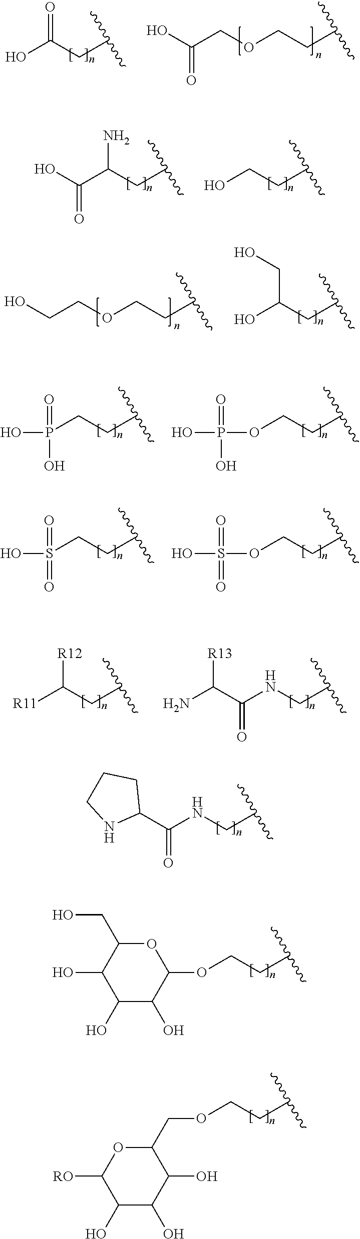

5. The compound according to claim 1, selected from the group consisting of the following compounds:

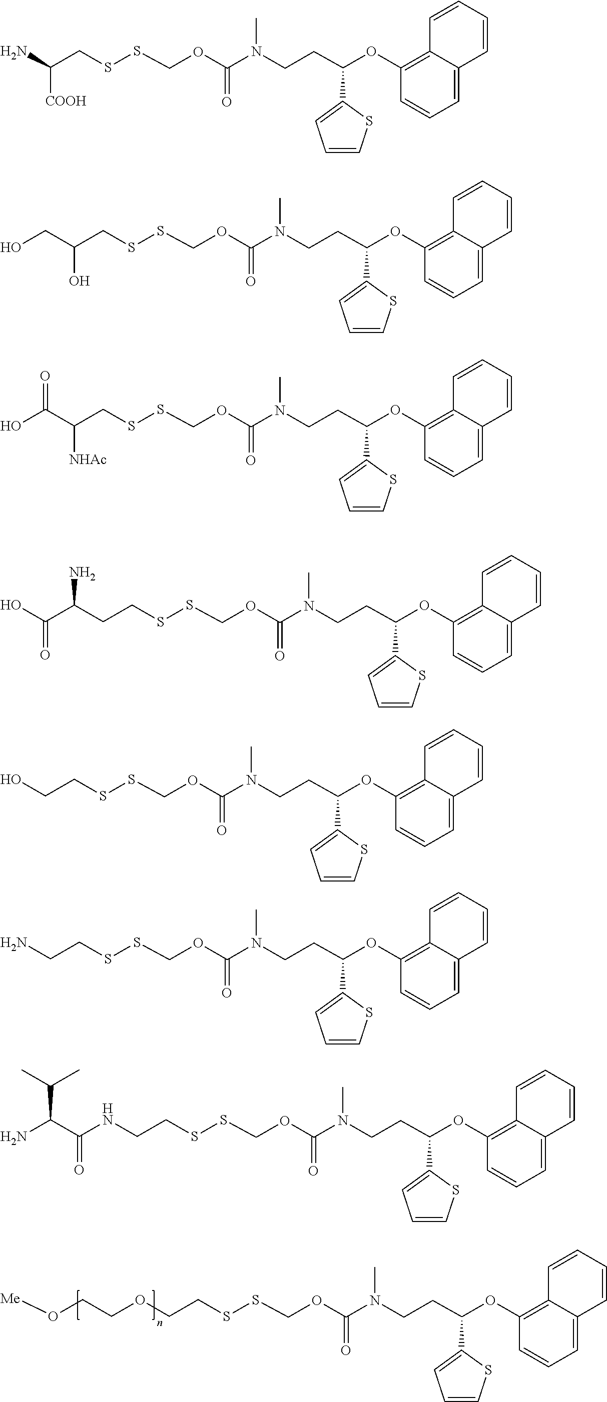

6. The compound according to claim 1, wherein G[C] is represented by Formula V: 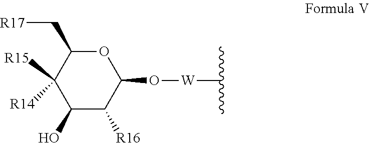
wherein W is selected from the group consisting of C1-20 (hetero)alkyl, —C(═O)N(R18)R19, —C(═O)NR20, and —C(═O)N(R18)-CH₂—O—(CH₂)ₘ—;
wherein R14 and R15 are each independently selected from the group consisting of OH, F, and H;
with the proviso that one of R14 and R15 is H and the other of R14 and R15 is OH or F;
wherein R16 is OH or F;
wherein R17 is selected from the group consisting of OH, F and H;
wherein R18 is selected from the group consisting of methyl, ethyl, n-propyl, isopropyl, n-butyl, sec-butyl, iso-butyl and 2-methoxyethyl;
wherein R19 is a C1-10 (hetero)alkyl;
wherein NR20 is a (hetero)cyclic structure; and
wherein m is an integer between 2 and 6.

7. The compound according to claim 6, in which O—W is selected from the group consisting of the following structures: 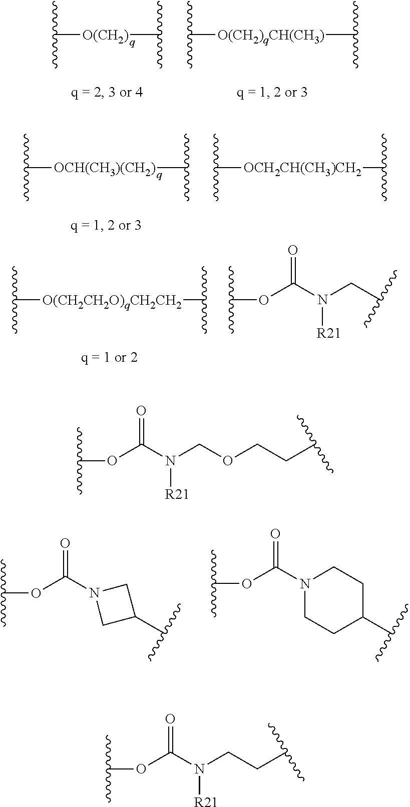
wherein R21 is selected from the group consisting of methyl, ethyl, n-propyl, isopropyl, n-butyl, isobutyl, sec-butyl, and 2-methoxyethyl.

8. The compound according to claim 1, wherein G[C] is represented by Formula VI: 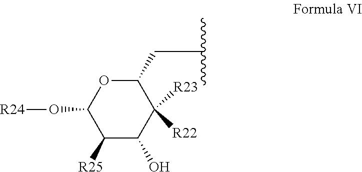
wherein R22 and R23 are each independently selected from the group consisting of OH, F, and H;
with the proviso that one of R22 and R23 is H and the other of R22 and R23 is OH or F;
wherein R25 is OH or F; and
wherein R24 is a C1-10 (hetero)alkyl or a compound according to Formula VII: 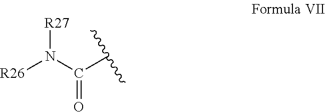
wherein R26 is H or a C1-C10 alkyl; and
wherein R27 is a C1-C10 alkyl.

9. A reagent compound according to Formula VIII: 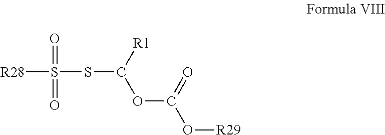
wherein R28 is methyl or 4-tolyl;
wherein R1 is selected from the group consisting of hydrogen, methyl, ethyl, n-propyl, isopropyl, n-butyl, sec-butyl, and t-butyl, preferably R1 is hydrogen or methyl; and
wherein R29 is pentafluorophenyl or 4-nitrophenyl.

10. A method for preparing a reagent compound according to claim 9, said method comprising the steps of:
i) reacting a 1-chloroalkyl chloroformate of formula ClC(═O)OCH(R1)Cl with pentafluorophenol when R29 is pentafluorophenyl or with 4-nitrophenol when R29 is 4-nitrophenyl in the presence of a base, to give the corresponding substituted phenyl chloromethyl carbonate;
ii) reacting the substituted phenyl chloromethyl carbonate obtained in step i) with sodium iodide in the presence of a base, to give a substituted phenyl iodomethyl carbonate; and
iii) reacting the substituted phenyl iodomethyl carbonate obtained in step ii) with an alkali methanethiosulfonate, preferably sodium methanethiosulfonate, when R28 is methyl or alkali p-toluenethiosulfonate, preferably potassium p-toluenethiosulfonate, when R28 is 4-tolyl to give the reagent compound of Formula VIII;
preferably wherein steps i), ii), and iii) are carried out under an inert atmosphere.

11. A method for preparing a compound according to Formula Ia or a pharmaceutically acceptable salt thereof: 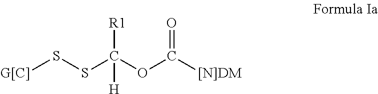
wherein each solid line represents a covalent bond, wherein H is hydrogen, O is oxygen, C is carbon, S is sulfur, and C═O is a carbonyl group;
wherein R1 is selected from the group consisting of hydrogen, methyl, ethyl, n-propyl, isopropyl, n-butyl, sec-butyl, and t-butyl, preferably R1 is hydrogen or methyl; and
wherein G is an organic structure and [C] represents a carbon atom of G;
wherein DM is a drug moiety and [N] is a nitrogen atom representing a part of DM;
said method comprising the steps of:
a) providing a reagent compound according claim 9;
b) reacting the reagent compound provided in step a) with a drug molecule [NH]DM in the presence of a base, [NH] represents a part of DM, with the proviso that [NH] is not part of an amide, carbamate or urethane to prepare an intermediate compound according to Formula IX: 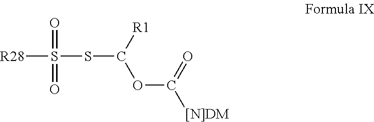
and c) reacting the intermediate compound of Formula IX obtained in step b) with G[C]—SH in the presence of a base, to provide the compound according to Formula Ia;
preferably wherein steps a), b), and c) are carried out under an inert atmosphere.

12. A method for preparing a compound according to claim 1, said method comprising the steps of:
A) contacting a drug molecule [ZH]DM, [ZH] represents a part of DM, wherein [ZH] is selected from the group consisting of an alcohol, phenol, oxime, a primary amine, secondary amine and a thiol, with the proviso that NH and NH₂ are not part of an amide, carbamate or urethane with a 1-chloroalkyl chloroformate of formula ClC(═O)OCH(R1)Cl in the presence of a base, to obtain an intermediate compound according to Formula X 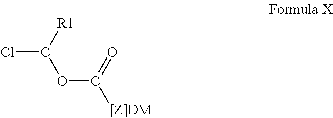
wherein R1 is selected from the group consisting of hydrogen, methyl, ethyl, n-propyl, isopropyl, n-butyl, sec-butyl, and t-butyl, preferably R1 is hydrogen or methyl;
B) contacting said intermediate compound according to Formula X obtained in step A) with an alkali methanethiosulfonate when R28 is methyl or with an alkali p-toluenethiosulfonate when R28 is 4-tolyl in order to obtain an intermediate compound according to Formula XI: 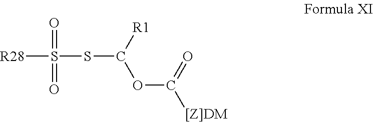
and C) reacting the intermediate compound of Formula XI obtained in step B) with G[C]—SH in the presence of a base, to provide the compound according to Formula I;
preferably wherein steps A), B), and C) are carried out under an inert atmosphere.

13. The method according to claim 11, wherein [ZH]DM is selected from the group consisting of from 5′-Deoxy-5-fluorocytidine, Cytarabine, Lenalidomide, Thalidomide, Acyclovir, Doxorubicin, Losartan, Ciclopirox, Albendazole, Duloxetine, Mesalazine, Linagliptin, Atomoxetine, 5-Fluorouracil, Methylphenidate, Palbociclib, Azacitidine, Gabapentin, Metoprolol, Nintedanib, Carvedilol, Gemcitabine, Rasagiline, Pscilocin, Celecoxib, Ibrutinib, Riluzole, Meropenem, Cinacalcet, Lapatinib, Tamiflu, Ceftriaxon, Abiraterone, Fesoterodine, Rotigotine, Orciprenaline, Acyclovir, Fulvestrant, Tenofovir, Ganciclovir, Testosterone, Kalydeco, Tizoxanide, Cannabidiol, Paliperidone, Venlafaxine, Edaravone, Paracetamol, Vorinostat, gemcitabine, Paclitaxel, Estradiol, 17-Ethynyl-estradiol, Propofol, Mercaptopurin, Acetylcysteine, Bucillamine, Captopril, and Zofenoprilat.

14. The compound according to claim 1 for use as a prodrug, a mutual prodrug, or an antedrug.

15. The compound according to claim 1 for use as a medicament, therapy, imaging agent or diagnostic agent.

16. The use of the compound according to claim 1 to improve one or more of the following properties of the drug that is present in said compound as drug moiety DM: solubility, permeability, stability, taste, oral bioavailability, dissolution and/or disposition.
